(19) United States
(12) Patent Application Publication
(10) Pub. No.: US 2024/0417743 A1
(43) Pub. Date: Dec. 19, 2024

(54) METHODS FOR IMPROVING GENOME ENGINEERING AND REGENERATION IN PLANT

(71) Applicant: KWS SAAT SE & Co. KGaA, Einbeck (DE)

(72) Inventor: Ling MENG, St. Louis, MO (US)

(73) Assignee: KWS SAAT SE & Co. KGaA, Einbeck (DE)

(21) Appl. No.: 18/740,126

(22) Filed: Jun. 11, 2024

Related U.S. Application Data
(62) Division of application No. 17/251,633, filed on Dec. 11, 2020, now Pat. No. 12,043,837.
(60) Provisional application No. 62/728,445, filed on Sep. 7, 2018.
(60) Provisional application No. 62/685,626, filed on Jun. 15, 2018.

(51) Int. Cl. C12N 15/82; C07K 14/415

(52) U.S. Cl. CPC C12N 15/8213; C07K 14/415; C12N 15/8207

(57) ABSTRACT

This document relates to methods and materials for genome engineering in eukaryotic cells, and particularly to methods for increasing genome engineering (i.e. transformation or genome editing) efficiency via delivery of one or more booster polypeptides, and boost genes, with genome engineering components.

### CROSS-REFERENCE TO RELATED APPLICATIONS

This application is a divisional of U.S. application Ser. No. 17/251,633, filed Dec. 11, 2020, which is a U.S. National Phase of International Patent Application No. PCT/EP2019/065645, filed on Jun. 14, 2019, which claims priority to U.S. Application No. 62/685,626, filed Jun. 15, 2018, and U.S. Application No. 62/728,445, filed Sep. 7, 2018. The entire contents of these applications are incorporated herein by reference in their entirety.

### SEQUENCE LISTING

The instant application contains a Sequence Listing which has been submitted electronically in XML format and is hereby incorporated by reference in its entirety. Said XML copy, created on Jun. 6, 2024, is named 245761_000241_SL.xml and is 203,742 bytes in size.

### TECHNICAL FIELD

Described herein are novel regeneration booster genes and polypeptides as well as methods and materials for genome engineering in eukaryotic cells, and particularly methods for increasing genome engineering (i.e., transformation or genome editing) efficiency via delivery of booster polypeptides, and boost genes, with genome engineering components.

### BACKGROUND OF THE INVENTION

Traditional breeding has provided domesticated plants and animals, while modern biotechnology, in particular genome engineering, is expanding breeding capability and enabling improvements that are not possible with only traditional crossing of close species. Using biotechnology, various traits, such as high-yield, herbicide tolerance and pest resistance, have been introduced into crops, resulting in dramatic advances in global agriculture and food security. However, the presence of foreign DNA in such products of biotechnology can trigger biosafety and environmental concerns.

By segregating out any integrated DNA, genome-editing technology can be used to generate a site-specific modification of the target genome without the presence of foreign DNA in the end plants. Moreover, by transient expression, genome editing can involve transient editing activity to create site-specific modification without DNA integration at any points of process. The genome-edited plants, especially those derived from the transient activity, would be significantly different from the conventional genome modified plants, and may not be regulated as genetically modified (GM) plants. Genome editing techniques, especially via a transient editing approach, thus can provide a highly accurate, safe and powerful plant breeding and development tool in agriculture.

Genome engineering based on transient activity however faces more challenges. Compared with stable transformation, transient engineering generally results in fewer modified cells. Without an integrated selectable marker, it is highly challenging to identify the engineered cells and achieve homogenous modification in the regenerated plants. These challenges stand in the way of routine implementation of transient gene editing as a breeding tool for plant improvement. Novel methods and materials that enhance genome engineering efficiency are thus highly desirable.

### SUMMARY OF THE INVENTION

In one aspect is provided a (regeneration) booster polypeptide comprising an amino acid sequence of SEQ ID NO: 2 or 48, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48.

In another aspect is provided a nucleic acid encoding a booster polypeptide comprising an amino acid sequence of SEQ ID NO: 2 or 48, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48. In some embodiments, the nucleic acid encoding a booster polypeptide comprising an amino acid sequence of SEQ ID NO: 2 an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 comprises a coding sequence selected from the group consisting of: (i) a nucleic acid comprising the nucleotide sequence of SEQ ID NO: 1; (ii) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1; and (iii) a nucleic acid hybridizing with the complementary strand of the nucleic acid as defined in (i) or (ii) under stringent hybridization conditions. In some embodiments, the nucleic acid encoding a booster polypeptide comprising an amino acid sequence of SEQ ID NO: 48 or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 48 comprises a coding sequence selected from the group consisting of: (I) a nucleic acid comprising the nucleotide sequence of SEQ ID NO: 47; (II) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 47; and (III) a nucleic acid hybridizing with the complementary strand of the nucleic acid as defined in (I) or (II) under stringent hybridization conditions.

In another aspect is provided a recombinant gene comprising a nucleic acid encoding a booster polypeptide comprising an amino acid sequence of SEQ ID NO: 2 or 48, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48. In some embodiments, the nucleic acid encoding a booster polypeptide comprising an amino acid sequence of SEQ ID NO: 2 or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2, comprises a coding sequence selected from the group consisting of: (i) a nucleic acid comprising the nucleotide sequence of SEQ ID NO: 1; (ii) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1; and (iii) a nucleic acid hybridizing with the complementary strand of the nucleic acid as defined in (i) or (ii) under stringent hybridization conditions. In some embodiments, the nucleic acid encoding a booster polypeptide comprising an amino acid sequence of SEQ ID NO: 48 or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 48 comprises a coding sequence selected from the group consisting of: (I) a nucleic acid comprising the nucleotide sequence of SEQ ID NO: 47; (II) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 47; and (III) a nucleic acid hybridizing with the complementary strand of the nucleic acid as defined in (I) or (II) under stringent hybridization conditions.

In some embodiments, the nucleic acid is operably linked to a heterologous promoter. The heterologous promoter can be a strong constitutive promoter, a tissue-specific promoter, a development-specific promoter, or an inducible promoter.

In another aspect is provided a DNA construct, preferably a vector, comprising any of the above nucleic acids or recombinant genes. In some embodiments, the nucleic acid comprises a coding sequence selected from the group consisting of: (i) a nucleic acid comprising the nucleotide sequence of SEQ ID NO: 1 or 47; (ii) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47; and (iii) a nucleic acid hybridizing with the complementary strand of the nucleic acid as defined in (i) or (ii) under stringent hybridization conditions.

In another aspect is provided a plant cell comprising the above booster polypeptides, nucleic acids, recombinant genes or DNA constructs, particularly as transgene or as heterologous polypeptide or heterologous nucleic acid. In some embodiments, the booster polypeptide comprises the amino acid sequence of SEQ ID NO: 2 or 48. In some embodiments, the booster polypeptide comprises the amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48. In some embodiments, the nucleic acid comprises a coding sequence selected from the group consisting of: (i) a nucleic acid comprising the nucleotide sequence of SEQ ID NO: 1 or 47; (ii) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47; and (iii) a nucleic acid hybridizing with the complementary strand of the nucleic acid as defined in (i) or (ii) under stringent hybridization conditions.

Also provided is a plant, a part of the plant, a seed, an embryo or a callus comprising the plant cell.

In another aspect is provided a method for genetic modification in a plant cell. The method comprises: (a) introducing into the plant cell (i) any of the above booster polypeptides, nucleic acids, recombinant genes or DNA constructs; and (ii) a transgene and/or a genome engineering component; (b) optionally, cultivating the plant cell under conditions allowing the synthesis of the booster polypeptide from the nucleic acid, the recombinant gene or the DNA construct; and (c) optionally, cultivating the plant cell under conditions allowing the genetic modification of the genome of said plant cell by integration of the transgene of interest and activity of the genome engineering component in the presence of the booster polypeptide.

In some embodiments, the booster polypeptide is transiently present, transiently active and/or transiently expressed in the plant cell. In some embodiments, the nucleic acid encoding the booster polypeptide is transiently present, transiently active and/or transiently expressed in the plant cell.

In step (i) of the method for genetic modification in a plant cell additionally one or more polypeptides selected from the group consisting of a PLT5 polypeptide, a PLT7 polypeptide, an RKD4 polypeptide, and an RKD2 polypeptide, and/or one or more nucleic acids selected from the group consisting of a nucleic acid encoding a PLT5 polypeptide, a PLT7 polypeptide, an RKD4 polypeptide, and an RKD2 polypeptide, and/or one or more site-directed transcriptional activators suitable to increase transiently the expression of an endogenous PLT5 polypeptide, an endogenous PLT7 polypeptide, an endogenous RKD4 polypeptide, or an endogenous RKD2 polypeptide, and/or a nucleic acid encoding such site-directed transcriptional activator are introduced into the plant cell.

In some embodiments, the PLT5 polypeptide or the PLT7 polypeptide is transiently present, transiently active and/or transiently expressed in the plant cell, or the nucleic acid encoding the PLT5 polypeptide or the PLT7 polypeptide is transiently present, transiently active and/or transiently expressed in the plant cell.

In some embodiments, both the booster polypeptide or the nucleic acid encoding the booster polypeptide, and the PLT5 polypeptide or the nucleic acid encoding the PLT5 polypeptide are introduced or co-delivered into the plant cell, preferably the same plant cell, and optionally transiently co-expressed. In some embodiments, both the booster polypeptide or the nucleic acid encoding the booster polypeptide, and the PLT7 polypeptide or the nucleic acid encoding the PLT7 polypeptide are introduced into the plant cell, and optionally transiently co-expressed.

In some embodiments, the PLT5 polypeptide comprises the amino acid sequence of SEQ ID NO: 4 or 6, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4 or 6, or the nucleic acid encoding the PLT5 polypeptide encodes such polypeptides. The PLT7 polypeptide comprises the amino acid sequence of SEQ ID NO: 8 or 10, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 8 or 10, or the nucleic acid encoding the PLT7 polypeptide encodes such polypeptides. In some embodiments, the RKD4 polypeptide comprises the amino acid sequence of SEQ ID NO: 12, 14 or 16, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 12, 14 or 16, or the nucleic acid encoding the RKD4 polypeptide encodes such polypeptides. In some embodiments, the RKD2 polypeptide comprises the amino acid sequence of SEQ ID NO: 18, 20 or 22, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 18, 20 or 22, or the nucleic acid encoding the RKD2 polypeptide encodes such polypeptides.

In some embodiments, the nucleic acid encoding the PLT5 polypeptide comprises a nucleic acid having a coding sequence selected from the group consisting of: (i) a nucleic acid comprising the nucleotide sequence of SEQ ID NO: 3 or 5; (ii) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 3 or 5; and (iii) a nucleic acid hybridizing with the complementary strand of the nucleic acid as defined in (i) or (ii) under stringent hybridization conditions. In some embodiments, the nucleic acid encoding the PLT7 polypeptide comprises a nucleic acid having a coding sequence selected from the group consisting of: (I) a nucleic acid comprising the nucleotide sequence of SEQ ID NO: 7 or 9; (II) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7 or 9; and (III) a nucleic acid hybridizing with the complementary strand of the nucleic acid as defined in (I) or (II) under stringent hybridization conditions. In some embodiments, the nucleic acid encoding the RKD4 polypeptide comprises a nucleic acid having a coding sequence selected from the group consisting of: (1) a nucleic acid comprising the nucleotide sequence of SEQ ID NO: 11, 13, or 15; (2) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 11, 13, or 15; and (3) a nucleic acid hybridizing with the complementary strand of the nucleic acid as defined in (1) or (2) under stringent hybridization conditions. In some embodiments, the nucleic acid encoding the RKD2 polypeptide comprises a nucleic acid having a coding sequence selected from the group consisting of: a) a nucleic acid comprising the nucleotide sequence of SEQ ID NO: 17, 19, or 21; b) a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 17, 19, or 21; and c) a nucleic acid hybridizing with the complementary strand of the nucleic acid as defined in a) or b) under stringent hybridization conditions.

In some embodiments, the genome engineering component comprises a) an enzyme inducing a double-stranded break (DSB) or a nucleic acid encoding same, and optionally a repair nucleic acid molecule, wherein the DSB-inducing enzyme preferably recognizes a predetermined site in the genome of said cell; b) an enzyme inducing a single-stranded break (SSB) or a nucleic acid encoding same, and optionally a repair nucleic acid molecule, wherein the SSB-inducing enzyme preferably recognizes a predetermined site in the genome of said cell; c) a base editor enzyme, optionally fused to a disarmed DSB- or SSB-inducing enzyme, wherein the base editor enzyme preferably recognizes a predetermined site in the genome of said cell; or d) an enzyme effecting DNA methylation, histone acetylation, histone methylation, histone ubiquitination, histone phosphorylation, histone ribosylation or histone citrullination, optionally fused to a disarmed DSB- or SSB-inducing enzyme, wherein the enzyme preferably recognizes a predetermined site in the genome of said cell.

In some embodiments, the genome engineering component comprising a DSB- or SSB-inducing enzyme or a variant thereof is a CRISPR/Cas endonuclease, a CRISPR/Cas9 endonuclease, a CRISPR/Cpf1 endonuclease, a CRISPR/Csm1 endonuclease, a zinc finger nuclease (ZFN), a homing endonuclease, a meganuclease, or a TAL effector nuclease.

In some embodiments, the activity of the genome engineering component in step (b) comprises inducing one or more double-stranded breaks in the genome of the plant cell, one or more single strand breaks in the genome of the plant cell, one or more base editing events in the genome of the plant cell, or one or more of DNA methylation, histone acetylation, histone methylation, histone ubiquitination, histone phosphorylation, histone sumoylation, histone ribosylation or histone citrullination in the genome of the plant cell.

In some embodiments, the induction of one or more double-stranded breaks or one or more single strand breaks is followed by non-homologous end joining (NHEJ) and/or by homology directed repair of the break(s) though a homologous recombination mechanism (HDR).

In some embodiments, the transgene in step (a) (ii) is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In some embodiments, in step (c) the modification of said genome is selected from i) a replacement of at least one nucleotide; ii) a deletion of at least one nucleotide; iii) an insertion of at least one nucleotide; iv) a change of the DNA methylation; v) a change in histone acetylation, histone methylation, histone ubiquitination, histone phosphorylation, histone sumoylation, histone ribosylation or histone citrullination; or vi) any combination of i)-v).

In some embodiments, the method is effective to promote cell proliferation or cell regeneration, preferably after genetic modification/modification of the genome or is effective to increase the efficiency for regeneration of transgenic, gene edited or base edited plants.

In some embodiments, the method is effective to induce direct or indirect embryogenesis from a single cell, preferably an embryonic cell, a somatic cell or a protoplast, or from a callus cell, preferably after genetic modification/modification of the genome.

In some embodiments, the method is effective to increase the stable transformation efficiency of the transgene into the plant cell or is effective to increase the efficiency for generation of transgenic plants.

In some embodiments, the method is effective to increase the efficiency of the genome engineering component to edit the genome of the plant cell or is effective to increase the efficiency for generation of transgenic, gene edited or base edited plants.

In some embodiments, the method is effective to improve the efficiency of regeneration of plants derived from recalcitrant genotypes, is effective to improve the efficiency of regeneration of plants from non-conventional tissue types, or is effective to accelerate the regeneration process, preferably after genetic modification/modification of the genome.

In some embodiments, the site-directed transcriptional activator, or the nucleic acid encoding the same, comprises at least one recognition domain and at least one activation domain, wherein the site-directed transcriptional activator is configured to increase the expression of an endogenous PLT5 polypeptide, an endogenous PLT7 polypeptide, an endogenous RKD4 polypeptide, or an endogenous RKD2 polypeptide, preferably by binding to a regulation region located at a certain distance in relation to the start codon of the endogenous PLT5 polypeptide, the endogenous PLT7 polypeptide, the endogenous RKD4 polypeptide, or the endogenous RKD2 polypeptide.

In some embodiments, the at least one recognition domain is, or is a fragment of, a molecule selected from the group consisting of at least one TAL effector, at least one disarmed CRISPR/nuclease system, at least one Zinc-finger domain, and at least one disarmed homing endonuclease, or any combination thereof. In some embodiments, the at least one disarmed CRISPR/nuclease system is selected from a CRISPR/dCas9 system, a CRISPR/dCpf1 system, a CRISPR/dCsm1 system, a CRISPR/dCasX system or a CRISPR/dCasY system, or any combination thereof, wherein the at least one disarmed CRISPR/nuclease system comprises at least one guide RNA. In some embodiments, the at least one activation domain is an acidic transcriptional activation domain, preferably, wherein the at least one activation domain is from an TAL effector gene of Xanthomonas oryzae, VP16 or tetrameric VP64 from Herpes simplex, VPR, SAM, Scaffold, Suntag, P300, VP160, or any combination thereof.

In another aspect is provided a method for improving the efficiency of plant regeneration or increasing the regeneration ability of a plant cell, the method comprising introducing into the plant cell any of the above booster polypeptides, nucleic acids, recombinant genes or DNA constructs.

In another aspect is provided a genetically modified plant cell obtained or obtainable according to the above methods. Also provided is a plant or a plant part comprising the genetically modified plant cell.

In another aspect is provided a microparticle coated with at least one of the above booster polypeptides, nucleic acids, recombinant genes or DNA constructs. In some embodiments, the microparticle is further coated with a genome engineering component.

In another aspect is provided a kit for the genetic modification of a plant genome by microprojectile bombardment, the kit comprising (I) one or more microparticles, and (II) means for coating the microparticles. In some embodiments, the kit further comprises a means for coating the microparticles with a genome engineering component.

In another aspect is provided a method for producing a genetically modified plant, comprising the steps: (a) genetically modifying a plant cell according to any of the above methods, and (b) regenerating a plant from the modified plant cell of step (a).

In some embodiments, the produced plant does not contain any of the genome engineering component, the boost gene, and the booster polypeptide, co-introduced in step (a).

In another aspect is provided a genetically modified plant or a part thereof obtained or obtainable by the above methods for producing a genetically modified plant, or a progeny plant thereof.

Also provided is a use of the above booster polypeptides, nucleic acids, recombinant gene, DNA construct, microparticle or kit for improving the efficiency of plant regeneration or increasing the regeneration ability of a plant cell.

### BRIEF DESCRIPTION OF THE DRAWINGS

FIG. 1 shows a map of the Boost gene expression vector pABM-BdEF1 (SEQ ID NO: 24). BdEF1 and nos-T define the strong constitutive promoter from Brachypodium EF1 gene and nos terminator, respectively. BamHI and HindIII illustrate the cloning sites.

FIG. 2 shows a map of the maize PLT5 expression construct pABM-BdEF1_ZmPLT5 (SEQ ID NO: 25). The maize PLT5 gene (ZmPLT5) is driven by the strong constitutive EF1 promoter from Brachypodium (pBdEF1).

FIG. 3 shows a map of the maize PLT7 expression construct pABM-BdEF1_ZmPLT7 (SEQ ID NO: 26). The maize PLT7 gene (ZmPLT7) is driven by the strong constitutive EF1 promoter from Brachypodium (pBdEF1).

FIG. 4 shows a map of the KWS-RBP1 expression construct pABM-BdEF1-KWS-RBP1 (SEQ ID NO: 27). KWS-RBP1 gene is driven by the strong constitutive EF1 promoter from Brachypodium (pBdEF1).

FIG. 5 shows a map of the wheat RKD4 expression construct pABM-BdEF1-TaRKD4 (SEQ ID NO: 28). The wheat RKD4 (TaRKD4 gene is driven by the strong constitutive EF1 promoter from Brachypodium (pBdEF1).

FIG. 6 shows a map of the genome editing CRISPR Cpf1 expression construct pGEP359 (SEQ ID NO: 29). tDTomato defines tdTomato gene (tDT). ZmLpCpf1 defines the maize codon-optimized CDS of the Lachnospiraceae bacterium CRISPR/Cpf1 (LbCpf1) gene.

FIG. 7 shows a map of the genome editing CRISPR RNA construct pGEP324 (SEQ ID NO: 30). crGEP05 defines the crRNA5 that targets to maize HMG13 gene. ZmUbi1 defines the promoter and intron from maize Ubiquitin 1 gene. Tnos defines the nos terminator.

FIG. 8 shows a Fluorescent image of A188 immature embryos 18 hours after co-bombardment of ZmPLT5 (FIG. 2) with pGEP359 (FIG. 6) and pGEP324 (FIG. 7) plasmids. Images were taken 18 hours after bombardment.

FIG. 9 shows transient co-expression of ZmPLT5 and KWS-RBP1 or ZmPLT7 and KWS-RBP1 promoting embryogenesis in Hi II immature embryos. Images show embryogenic structures induced from maize Hi II embryos 5 days after co-bombardment with boost gene constructs. FIG. 9A shows bombardment of genome engineering constructs pGEP359 (FIG. 6) and pGEP324 (FIG. 7) only (GE constructs only). FIG. 9B shows co-delivery of ZmPLT5 (FIG. 2) and KWS-RBP1 (FIG. 4) with the GE constructs (GE constructs plus ZmPLT5+KWS-RBP1). FIG. 9C shows co-delivery of ZmPLT7 and KWS-RBP1 with the GE constructs (GE constructs plus ZmPLT7+KWS-RBP1). Images were taken 5 days after bombardment.

FIG. 10 shows transient co-expression of ZmPLT5 and KWS-RBP1 or ZmPLT7 and KWS-RBP1 promotes stable transformation of the co-delivered tDT report gene in maize Hi II embryo. Red fluorescence images show stable tDT expressing structures produced from maize Hi II embryos 12 days after co-bombardment (FIGS. 10A to 10C). FIG. 10A shows bombardment of genome engineering constructs pGEP359 (FIG. 6) and pGEP324 (FIG. 7) only (GE constructs only). FIG. 10B shows co-delivery of ZmPLT5 and KWS-RBP1 with the GE constructs (GE constructs plus ZmPLT5+KWS-RBP1). FIG. 10C shows co-delivery of ZmPLT7 and KWS-RBP1 with the GE constructs (GE constructs plus ZmPLT7+KWS-RBP1). FIG. 10D is a graph showing that co-delivery of ZmPLT5 or ZmPLT7 and KWS-RBP1 increased stable transformation frequency of the tDT report gene. Results were taken 12 days after bombardment.

FIG. 11 shows transient co-expression of ZmPLT5 and KWS-RBP1 or ZmPLT7 and KWS-RBP1 promotes embryogenesis in A188 immature embryos. Images show embryogenic structures induced from maize A188 embryos 7 days after co-bombardment with boost gene constructs. FIG. 11A shows bombardment of genome engineering constructs pGEP359 (FIG. 6) and pGEP324 (FIG. 7) only (GE constructs only); FIG. 11B shows co-delivery of ZmPLT5 and KWS-RBP1 with the GE constructs (GE constructs plus ZmPLT5+KWS-RBP1); FIG. 11C shows co-delivery of ZmPLT7 and KWS-RBP1 with the GE constructs (GE constructs plus ZmPLT7+KWS-RBP1). Images were taken 7 days after bombardment.

FIG. 12 shows transient co-expression of ZmPLT5 and KWS-RBP1 promotes stable transformation of the co-delivered tDT report gene in maize A188 embryo. Red fluorescence images show stable tDT expressing structures produced from maize A188 embryos 16 days after co-bombardment (A to C). FIG. 12A shows bombardment of genome engineering constructs pGEP359 (FIG. 6) and pGEP324 (FIG. 7) only (GE constructs only); FIG. 12B shows co-delivery of ZmPLT5 and KWS-RBP1 with the GE constructs (GE constructs plus ZmPLT5+KWS-RBP1); FIG. 12C shows co-delivery of ZmPLT7 and KWS-RBP1 with the GE constructs (GE constructs plus ZmPLT7+KWS-RBP1). FIG. 12D shows co-delivery of ZmPLT5 or ZmPLT7 and KWS-RBP1 increased stable transformation frequency of tDT report gene. Results were taken 12 days after bombardment.

FIG. 13 shows a map of the maize WUS2 (ZmWUS2) promoter report construct pAMK-ZmWUS2-tDT-nosT (SEQ ID NO: 43). tDTomato define the fluorescence tDT report gene, which is driven by maize WUSCHEL2 promoter (pZmWUS2).

FIG. 14 shows that wheat TaRKD4 gene activates maize WUS2 promoter by transient co-bombardment in maize immature embryos IE (top panel) and leaves (bottom panel). FIG. 14A shows a maize WUS2 promoter report construct (FIG. 13; SEQ ID NO: 46) only (pZmWUS2 report only). FIG. 14B shows co-bombardment of the maize WUS promoter report construct and wheat RKD4 construct (FIG. 5) (pZmWUS2 report and TaRKD4). Images were taken 44 hours after bombardment.

FIG. 15 shows transient co-expression of wheat RKD4 (TaRKD4) and KWS-RBP1 promotes embryogenesis in Hi II immature embryos. Images show embryogenic structures induced from maize Hi II embryos 5 days after co-bombardment with the boost gene constructs. FIG. 15A shows bombardment of genome engineering constructs pGEP359 (FIG. 6) and pGEP324 (FIG. 7) only (GE constructs only). FIG. 15B shows co-delivery of TaRKD4 and KWS-RBP1 with the GE constructs (GE constructs plus KWS_RGB1+TaRKD4). Images were taken 5 days after bombardment.

FIG. 16 shows transient co-expression of wheat RKD4 (TaRKD4) and KWS-RBP1 promotes stable transformation of the co-delivered tDT report gene in maize Hi II embryo. Red fluorescence images show stable tDT expressing structures produced from maize Hi II embryos 12 days after co-bombardment (FIGS. 16A to 16C). FIG. 16A shows bombardment of genome engineering constructs pGEP359 (FIG. 6) and pGEP324 (FIG. 7) only (GE constructs only). FIG. 16B shows co-delivery of TaRKD4 and KWS-RBP1 with the GE constructs (GE constructs plus TaRKD4+KWS-RGB1). FIG. 16C shows co-delivery of TaRKD4 and KWS-RBP1 increased stable transformation frequency of tDT report gene in Hi II immature embryos. Results were taken 12 days after bombardment.

FIG. 17 shows that transient co-expression of wheat RKD4 and KWS-RBP1 promotes embryogenesis in A188 immature embryos. Images show embryogenic structures induced from maize Hi II embryos 5 days after co-bombardment with boost gene constructs. FIG. 17A shows bombardment of genome engineering constructs pGEP359 (FIG. 6) and pGEP324 (FIG. 7) only (GE constructs only). FIG. 17B shows co-delivery of TaRKD4 and KWS-RBP1 with the GE constructs (GE constructs plus TaRKD4+KWS-RBP1). Images were taken 5 days after bombardment.

FIG. 18 shows transient co-expression of wheat RKD4 and KWS-RBP1 promotes stable transformation of co-delivered tDT report gene in maize A188 embryo. Red fluorescence images show stable tDT expressing structures produced from maize A188 embryos 14 after co-bombardment (A to C). FIG. 18A shows bombardment of genome engineering constructs pGEP359 (FIG. 6) and pGEP324 (FIG. 7) only (GE constructs only). FIG. 18B shows co-delivery of KWS-RBP1 and TaRKD4 with the GE constructs (GE constructs plus TaRKD4+KWS-RBP1). FIG. 18C shows co-delivery of TaRKD4 and KWS-RBP1 increased stable transformation frequency of tDT report gene in maize A188 immature embryos. Results were taken 14 days after bombardment.

FIG. 19 shows transient co-expression of ZmPLT5 or ZmPLT7 and KWS-RBP1 promotes transient genome editing in maize. The genome editing constructs pGEP359 and pGEP324 were co-bombarded with the boost gene constructs into maize Hi II immature embryos. Editing efficiency is defined as the number of plants with a site-specific modification from 100 plants regenerated. Transient editing is used to describe a site-specific modification that resulted from transient activity of genome editing without an integration of the genetic materials.

FIG. 20 illustrates Droplet Digital PCR results, which demonstrate homogenous genome editing in regenerated plants by transient co-expression of the boost genes and genome editing components without a selection. The site-specific InDel rates of around 50% and 100% indicate a mono-allelic and bi-allelic modification, respectively. FIG. 20A shows negative control results from Droplet Digital PCR using water (bottom panel) or the wild type DNA (WT droplets; top panel). FIG. 20B shows Droplet Digital PCR results from the edited T0 plants derived from transient co-expression of boosters and genome editing components. The top panel and middle panel show a near 100% InDel rate from two edited T0 plants, indicating homogenous bi-allelic modification, while the bottom panel illustrates a homogenous mono-allelic edited event.

FIG. 21 depicts a multiple sequence alignment of the target region from the edited T0 plants by Sanger sequencing analysis. FIGS. 21A and 21B show bi-allelic events CB0113-T-591 and CB0113-T-632, respectively. FIG. 21C shows mono-allelic event CB0113-T-303. The PAM and expected cleavage site are labeled. A SNP (G from A188 and A from B73 allele) near the PAM site (TTTA) is also marked. The sequencing results confirm the homogenous modification occurred in these T0 plants. Specifically, CB0113-T-591 harbors a biallelic modification of 5 bp and 2 bp deletion from A188 and B73 allele, respectively. CB0113-T-632 contains a biallelic editing of 6 bp and 5 bp deletion from A188 and B73 allele, respectively. CB0113-T-303 has an 8 bp deletion from A188 allele, while the B73 allele is unmodified. CB0113-T-591 and CB0113-T-632 are derived from co-expression of ZmPLT5 and KWS-RBP1, and CB0113-T-303 is from co-expression of ZmPLT7 and KWS-RBP1 with the genome editing constructs. FIG. 21 discloses SEQ ID NOS 51. 52. 52. 53. 53-58. 58, 59, and 59, respectively. in order of appearance.

FIG. 22 shows KWS-RBP2 expression construct (pABM-BdEF1_KWS_RBP2) map. KWS-RBP2 gene was maize-codon optimized from its protein sequence and synthesized by Integrated DNA Technologies (IDT, San Diego, CA, USA), and cloned into expression vector pABM-BdEF1 (FIG. 1) at the cloning site of BamHI and HindIII. pKWS-RBP2 gene is driven by the strong constitutive EF1 promoter from Brachypodium (pBdEF1).

FIG. 23 illustrates that co-delivery of ZmPLT5 and KWS-RBG1 or ZmPLT5 and KWS-RBP2 promotes regeneration rate in maize A188. Maize immature embryos were bombarded with genome engineering constructs pGEP359 (FIG. 6) and pGEP324 (FIG. 7) only (tDTonly) or co-bombarded with ZmPLT5 and KWS_RBP1 (tDT plus ZmPLT5 and KWS_RBP1) or with ZmPLT5 and KWS_RBP2 (tDT plus ZmPLT5 and KWS_RBP2).

FIG. 24 shows that co-delivery of ZmPLT5 and KWS_RBG1 or ZmPLT5 and KWS_RBP2 promotes stable transformation efficiency of tDTomato report gene in maize A188. Red fluorescence images show stable tDT expressing structures (bright spots/areas) produced from maize A188 embryos 10 days after co-bombardment (A to C). A: Bombardment of genome engineering (GE) constructs pGEP359 (FIG. 6) and pGEP324 (FIG. 7) only (tDTonly); B: Co-bombardment of the GE constructs with ZmPLT5 and KWS-RBP1 (tDT plus ZmPLT5 and KWS_RBP1); C: Co-bombardment of the GE constructs with ZmPLT5 and KWS-RBP2 (tDT plus ZmPLT5 and KWS_RBP2). Images were taken 10 days after bombardment.

FIG. 25 shows that co-delivery of ZmPLT5 and KWS-RBG1 or ZmPLT5 and KWS_RBP2 promotes stable transformation efficiency of tDTomato report gene in maize A188. Red fluorescence images show stable tDT expressing structures (bright spots/areas) produced from maize A188 embryos 16 days after co-bombardment (A to C). A: Bombardment of genome engineering (GE) constructs pGEP359 (FIG. 6) and pGEP324 (FIG. 7) only (tDTonly); B: Co-bombardment of the GE constructs with ZmPLT5 and KWS-RBP1 (tDT plus ZmPLT5 and KWS_RBP1); C: Co-bombardment of the GE constructs with ZmPLT5 and KWS-RBP2 (tDT plus ZmPLT5 and KWS_RBP2). D: Co-delivery of ZmPLT5 and KWS-RBP1 or ZmPLT5 and KWS-RBP2 increased stable transformation frequency of tDT report gene. Data was recorded 16 days after bombardment. Images were taken 16 days after bombardment.

### DETAILED DESCRIPTION

### Definitions

Unless defined otherwise, technical and scientific terms used herein have the same meaning as commonly understood by one of ordinary skill in the art to which this invention belongs.

As used in the context of the present application, the term “about” means +/−10% of the recited value, preferably +/−5% of the recited value. For example, about 100 nucleotides (nt) shall be understood as a value between 90 and 110 nt, preferably between 95 and 105 nt.

As used herein, the terms “booster”, “booster gene”, “booster polypeptide”, “boost polypeptide”, “boost gene” and “boost factor” refer to a protein/peptide(s) or a (poly)nucleic acid fragment encoding the protein/polypeptide causing improved genome engineering and/or improved plant regeneration of transformed or gene edited plant cells. Such protein/polypeptide may increase the capability or ability of a plant cell, preferably derived from somatic tissue, embryonic tissue, callus tissue or protoplast, to regenerate in an entire plant, preferably a fertile plant. Thereby, they may regulate somatic embryo formation (somatic embryogenesis) and/or they may increase the proliferation rate of plant cells. Exemplary booster polypeptides include, but are not limited to, KWS-RBP1 (e.g., SEQ ID NO: 2) and variants. A variant thereof is for example KWS-RBP2 (SEQ ID NO: 48) which has a sequence identity at amino acid sequence level of 93%. The regeneration of transformed or gene edited plant cells may include the process of somatic embryogenesis, which is an artificial process in which a plant or embryo is derived from a single somatic cell or group of somatic cells. Somatic embryos are formed from plant cells that are not normally involved in the development of embryos, i.e. plant tissue like buds, leaves, shoots etc. Applications of this process may include: clonal propagation of genetically uniform plant material; elimination of viruses; provision of source tissue for genetic transformation; generation of whole plants from single cells, such as protoplasts; development of synthetic seed technology. Cells derived from competent source tissue may be cultured to form a callus. Plant growth regulators like auxins or cytokinines in the tissue culture medium can be manipulated to induce callus formation and subsequently changed to induce embryos to form from the callus. Somatic embryogenesis has been described to occur in two ways: directly or indirectly. Direct embryogenesis occurs when embryos are started directly from explant tissue creating an identical clone. Indirect embryogenesis occurs when explants produced undifferentiated, or partially differentiated, cells (i.e. callus) which then is maintained or differentiated into plant tissues such as leaf, stem, or roots.

The term “transgenic” as used according to the present disclosure refers to a plant, plant cell, tissue, organ or material which comprises a gene or a genetic construct, comprising a “transgene” that has been transferred into the plant, the plant cell, tissue organ or material by natural means or by means of transformation techniques from another organism. The term “transgene” comprises a nucleic acid sequence, including DNA or RNA, or an amino acid sequence, or a combination or mixture thereof. Therefore, the term “transgene” is not restricted to a sequence commonly identified as “gene”, i.e. a sequence encoding protein. It can also refer, for example, to a non-protein encoding DNA or RNA sequence. Therefore, the term “transgenic” generally implies that the respective nucleic acid or amino acid sequence is not naturally present in the respective target cell, including a plant, plant cell, tissue, organ or material. The terms “transgene” or “transgenic” as used herein thus refer to a nucleic acid sequence or an amino acid sequence that is taken from the genome of one organism, or produced synthetically, and which is then introduced into another organism, in a transient or a stable way, by artificial techniques of molecular biology, genetics and the like. A “plant material” as used herein refers to any material which can be obtained from a plant during any developmental stage. The plant material can be obtained either in planta or from an in vitro culture of the plant or a plant tissue or organ thereof. The term thus comprises plant cells, tissues and organs as well as developed plant structures as well as sub-cellular components like nucleic acids, polypeptides and all chemical plant substances or metabolites which can be found within a plant cell or compartment and/or which can be produced by the plant, or which can be obtained from an extract of any plant cell, tissue or a plant in any developmental stage. The term also comprises a derivative of the plant material, e.g., a protoplast, derived from at least one plant cell comprised by the plant material. The term therefore also comprises meristematic cells or a meristematic tissue of a plant.

The term of “genome engineering” is used herein, refer to strategies and techniques for the genetic modification of any genetic information or genome of a plant cell, comprising genome transformation, genome editing. As such “genome editing” refers to techniques for the targeted, specific modification of any genetic information or genome of a plant cell. As such, the terms comprise gene editing gene encoding region, but also the editing of regions other than gene encoding regions of a genome. It further comprises the editing or engineering of the nuclear (if present) as well as other genetic information of a plant cell. Furthermore, “genome engineering” also comprises an epigenetic editing or engineering, i.e., the targeted modification of, e.g., methylation, histone modification or of non-coding RNAs possibly causing heritable changes in gene expression.

The term “genome editing” as used herein refers to strategies and techniques for the targeted, specific modification of any genetic information or genome of a plant cell. As such, the terms comprise gene editing, but also the editing of regions other than gene encoding regions of a genome, such as intronic sequences, non-coding RNAs, miRNAs, sequences of regulatory elements like promoter, terminator, transcription activator binding sites, cis or trans acting elements. Additionally, “genome editing” may comprise base editing for targeted replacement of single nucleobases. It can further comprise the editing of the nuclear genome as well as other genetic information of a plant cell, i.e. mitochondrial genome or chloroplast genome as well as miRNA, pre-mRNA or mRNA. Furthermore, “genome editing” may comprise an epigenetic editing or engineering, i.e., the targeted modification of, e.g., DNA methylation or histone modification, such as histone acetylation, histone methylation, histone ubiquitination, histone phosphorylation, histone sumoylation, histone ribosylation or histone citrullination, possibly causing heritable changes in gene expression. “Genome editing” may also comprise an epigenetic editing or engineering of non-coding RNAs possibly causing heritable changes in gene expression.

A “base editor” as used herein refers to a protein or a fragment thereof having the same catalytic activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor.

As used herein, a “regulatory element” refers to nucleotide sequences which are not part of the protein-encoding nucleotide sequence, but mediate the expression of the protein-encoding nucleotide sequence. Regulatory elements include, for example, promoters, cis-regulatory elements, enhancers, introns or terminators. Depending on the type of regulatory element it is located on the nucleic acid molecule before (i.e., 5′ of) or after (i.e., 3′ of) the protein-encoding nucleotide sequence. Regulatory elements are functional in a living plant cell. The term “operatively linked” means that a regulatory element is linked in such a way with the protein-encoding nucleotide sequence, i.e., is positioned in such a way relative to the protein-encoding nucleotide sequence on, for example, a nucleic acid molecule that an expression of the protein-encoding nucleotide sequence under the control of the regulatory element can take place in a living cell.

As used herein, “upstream” indicates a location on a nucleic acid molecule which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

As used herein, a “flanking region”, is a region of the repair nucleic acid molecule having a nucleotide sequence which is homologous to the nucleotide sequence of the DNA region flanking (i.e. upstream or downstream) of the preselected site.

As used herein, “transient expression” refers to the phenomenon where the transferred protein/polypeptide and nucleic acid fragment encoding the protein/polypeptide is expressed and/or active transiently in the cells, and turned off and/or degraded shortly with the cell growth.

As used herein, a “double-stranded DNA break inducing enzyme”, “enzyme inducing a double-stranded break”, or “DSBI enzyme” is an enzyme capable of inducing a double-stranded DNA break at a particular nucleotide sequence, called the “recognition site” or “predetermined site”. Accordingly, a “single-stranded DNA or RNA break inducing enzyme”, “enzyme inducing a single-stranded break”, or “SSBI enzyme” is an enzyme capable of inducing a single-stranded DNA or RNA break at a particular nucleotide sequence, called the “recognition site” or “predetermined site”.

As used herein, a “repair nucleic acid molecule” is a single-stranded or double-stranded DNA molecule or RNA molecule that is used as a template for modification of the genomic DNA or the RNA at the preselected site in the vicinity of or at the cleavage site. As used herein, “use as a template for modification of the genomic DNA”, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region).

As used herein, “a modification of the genome”, means that the genome has changed in at least one nucleotide or by at least one epigenetic editing.

As used herein “a preselected site”, “a predetermined site” or “predefined site” indicates a particular nucleotide sequence in the genome (e.g. the nuclear genome or the chloroplast genome) at which location it is desired to insert, replace and/or delete one or more nucleotides.

As used herein, “phytohormone” or “plant growth regulator” refers to any material and chemical, either naturally occurred or synthesized, which promotes plant cell division and/or plant morphogenesis. As used herein, “regeneration” refers to a process, in which single or multiple cells proliferate and develop into tissues, organs, and eventually entire plants.

As used herein, the terms “vector”, or “plasmid (vector)” refers to a construct comprising, inter alia, plasmids or (plasmid) vectors, cosmids, artificial yeast- or bacterial artificial chromosomes (YACs and BACs), phagemides, bacterial phage based vectors, an expression cassette, isolated single-stranded or double-stranded nucleic acid sequences, comprising sequences in linear or circular form, or amino acid sequences, viral vectors, including modified viruses, and a combination or a mixture thereof, for introduction or transformation, transfection or transduction into any eukaryotic cell, including a plant, plant cell, tissue, organ or material according to the present disclosure.

“Recombinant” in the context of the recombinant gene can comprise regulatory sequences and/or localization sequences. The recombinant construct or the DNA construct according to the present invention can be integrated into or can be a vector, including a plasmid vector, and/or it can be present isolated from a vector structure, for example, in the form of a single-stranded or double-stranded nucleic acid. After its introduction, e.g. by transformation or transfection by biological or physical means, the recombinant gene or the DNA construct can either persist extrachromosomally, i.e. non integrated into the genome of the target cell, for example in the form of a double-stranded or single-stranded DNA. Alternatively, the recombinant gene or the DNA construct, can be stably integrated into the genome of a target cell, including the nuclear genome or further genetic elements of a target cell, including the genome of plastids like mitochondria or chloroplasts.

### Booster Polypeptide and Nucleic Acid Encoding Booster Polypeptide

In one aspect is provided a booster polypeptide comprising an amino acid sequence of SEQ ID NO: 2 or 48 (e.g., KWS-RBP1 or KWS-RBP2), or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48.

The inventor shows that the booster polypeptides KWS-RBP1 and KWS-RBP2 mediate a strong booster effect alone but also in combination with other booster polypeptides, in particular in the early phase of regeneration after delivery of transgene and/or the genome engineering component. This boost effect does not compromise plant development and regenerated plants show favorable plant growth in the adult stage and are fertile. As such, integration of booster genes or booster polypeptides can be segregated out in the following generation by crossing and selection.

In the various methods disclosed herein, any single booster polypeptide or combination of booster polypeptides can be transiently provided or co-expressed. A booster polypeptide itself may be introduced into the plant cell, or alternatively a polynucleotide encoding for the booster polypeptide may be introduced into the plant cell. With respect to combinations, one of the booster polypeptides can be introduced into the plant cell, along with a nucleotide encoding for another booster polypeptide, or the same booster polypeptide. For example, a booster polypeptide comprising the sequence of SEQ ID NO: 2 can be introduced into a plant cell along with a nucleic acid comprising the nucleotide sequence of SEQ ID NO: 1 (which encodes for the sequence of SEQ ID NO: 2).

Sequence of KWS-RBP1 booster polypeptide
(SEQ ID NO: 2)
MESGSGTAAGSGYVYRQPGSTRWNPTAEQLSLLREIYYRNGLRTPTADEI
RQISSKLSRYGKIEGKNVYNWFQNRRAREKRKQRLSTIGCDPALIEMGNV
ASLEFGTESALESLSSGPSSELREAPTRKFYEKKTVGENSTIINPVEQNC
TLSCGTSQEFQYAVDSRRVMKAMEEKQATDDEPDGNKWTESNRHVKILQL
FPLHNNEDQTLIKSDKEIYCLGSCEKKMDLSPLGHSGSQRASALDLCLSL
GNESCGLHDN
Sequence of a nucleic acid encoding the KWS-RBP1
booster polypeptide
(SEQ ID NO: 1)
ATGGAGTCGGGCTCCGGGACGGCTGCTGGCTCTGGCTATGTTTACAGACA
GCCAGGATCAACGCGGTGGAACCCGACAGCTGAACAACTGTCCTTGCTTA
GAGAAATCTACTACCGCAACGGATTGCGGACCCCGACCGCGGACGAAATC
AGACAAATCAGCTCAAAGCTCTCAAGGTACGGAAAAATAGAGGGCAAAAA
CGTTTACAACTGGTTCCAGAATAGACGCGCAAGAGAAAAGCGCAAGCAAC
GGCTCTCTACAATCGGCTGTGATCCAGCACTGATCGAGATGGGGAATGTC
GCTTCACTGGAATTCGGTACTGAGAGCGCCCTGGAATCGCTGTCGTCAGG
ACCATCCTCAGAACTCCGCGAAGCGCCAACGAGAAAATTTTACGAAAAAA
AGACGGTTGGAGAGAACTCAACTATAATAAACCCAGTGGAACAAAACTGT
ACCCTTTCCTGCGGAACGTCCCAAGAGTTCCAGTATGCGGTCGATTCTCG
GCGCGTCATGAAAGCTATGGAGGAAAAGCAGGCGACGGACGATGAACCCG
ACGGAAATAAATGGACTGAGTCAAACAGACACGTCAAGATTCTCCAGCTT
TTCCCGCTCCACAATAACGAGGATCAGACATTGATAAAGAGCGACAAAGA
AATCTATTGTTTGGGCTCGTGCGAGAAGAAAATGGATTTGTCACCGCTGG
GTCATTCAGGCTCTCAGCGCGCTTCGGCCCTTGACTTGTGCCTTTCATTG
GGCAACGAATCTTGTGGGCTGCATGATAATTGA

In another example, a booster polypeptide comprising the sequence of SEQ ID NO: 48 can be introduced into a plant cell along with a nucleic acid comprising the nucleotide sequence of SEQ ID NO: 47 (which encodes for the sequence of SEQ ID NO: 48).

Sequence of KWS-RBP2 booster polypeptide
(SEQ ID NO: 48)
MESGSGTAAGSGYVYRQSGSTRWNPTAEQLSLLKELYYRNGIRTPSADQI
RQISARLSRYGKIEGKNVFYWFQNHKARERQKKRLSTVGCDPALIEMGNV
ASLEFGTESALESLSSGPSSELREAPTRKFYEKKTVGENSTIINPVEQNC
TLSCGTSQEFQYAVDSRRVMKAMEEKQATDDEPDGNKWTESNRHVKTLPL
FPLHNNEDQTLIKSDKEIYCLGSCEKKMDLSPLGHSGSQRASALDLCLSL
GNESCGLHDN
Sequence of a nucleic acid encoding the KWS-RBP2
booster bolybebtide
(SEQ ID NO: 47)
ATGGAATCGGGCTCCGGCACGGCGGCAGGGTCTGGTTATGTCTATCGGCA
GAGCGGAAGCACCCGGTGGAATCCAACAGCAGAACAGTTGTCGCTGCTCA
AGGAACTTTATTACCGGAATGGAATTCGGACACCGTCGGCAGATCAAATT
AGGCAAATTTCGGCCCGGCTGTCCAGATACGGCAAAATAGAAGGGAAAAA
CGTCTTTTACTGGTTTCAAAATCATAAAGCACGGGAACGGCAGAAGAAAA
GACTTTCCACGGTCGGCTGCGACCCTGCTCTCATAGAAATGGGTAACGTC
GCGAGCTTGGAATTTGGGACCGAAAGCGCTCTTGAATCTCTCAGCTCAGG
CCCGTCCAGCGAGTTGCGCGAGGCTCCTACCCGCAAGTTTTATGAGAAGA
AAACCGTTGGTGAGAACAGCACCATAATCAATCCTGTTGAGCAGAACTGC
ACACTTTCTTGCGGTACTTCGCAGGAATTTCAGTATGCTGTTGATAGCCG
CCGGGTGATGAAGGCAATGGAAGAGAAGCAAGCAACGGATGATGAACCGG
ACGGAAACAAATGGACGGAGTCGAACAGGCATGTGAAGACCCTCCCTCTT
TTCCCCTTGCATAATAATGAAGATCAGACCTTGATCAAGTCGGACAAGGA
AATTTATTGCCTTGGGAGCTGTGAAAAAAAAATGGATCTGTCCCCATTGG
GACACTCGGGCTCTCAGAGGGCGTCGGCACTGGATTTGTGCCTGTCTTTG
GGTAATGAATCTTGTGGCCTCCACGACAATTGA

Also provided is a nucleic acid encoding a booster polypeptide comprising an amino acid sequence of SEQ ID NO: 2 or 48. Further provided is a nucleic acid encoding a booster polypeptide comprising an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48.

The nucleic acid encoding a booster polypeptide comprising an amino acid sequence of SEQ ID NO: 2 or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2, can comprise a nucleic acid comprising the nucleotide sequence of SEQ ID NO: 1. The nucleic acid can comprise a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1. Alternatively, the nucleic acid can hybridize, under stringent hybridization conditions, with the complementary strand of a nucleic acid comprising the nucleotide sequence of SEQ ID NO: 1 or a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1.

The nucleic acid encoding a booster polypeptide comprising an amino acid sequence of SEQ ID NO: 48 or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 48, can also comprise a nucleic acid comprising the nucleotide sequence of SEQ ID NO: 47. The nucleic acid can comprise a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 47. Alternatively, the nucleic acid can hybridize, under stringent hybridization conditions, with the complementary strand of a nucleic acid comprising the nucleotide sequence of SEQ ID NO: 47 or a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 47.

A recombinant gene comprising a nucleic acid encoding a booster polypeptide comprising an amino acid sequence of SEQ ID NO: 2 or 48, or an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48 is provided. The nucleic acid can be operatively linked to one or more regulatory elements. The regulatory element can be a promoter, a cis-regulatory element, an enhancer, an intron or a terminator. The regulatory element can be 5′ to the nucleic acid sequence. The regulatory element can be 3′ to the nucleic acid sequence. The nucleic acid can comprise a nucleic acid comprising the nucleotide sequence of SEQ ID NO: 1 or 47. The nucleic acid can comprise a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47. The nucleic acid can hybridize, under stringent hybridization conditions, with the complementary strand of a nucleic acid comprising the nucleotide sequence of SEQ ID NO: 1 or 47 or a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47.

In some embodiments, the nucleic acid is operably linked to a heterologous promoter. The heterologous promoter can be a strong constitutive promoter (such as a doubled 35S promoter (d35S)), a tissue-specific promoter, a development-specific promoter, or an inducible promoter. The heterologous promoter can be the promoter from the EF1 gene (such as the Brachypodium EF1 gene (pBdEF1, SEQ ID NO: 23), the promoter from a Ubiquitin 1 gene (such as the maize Ubiquitin 1 gene), a WUSCHEL2 promoter (such as the maize WUSHCEL2 promoter (pZmWUS2)). The heterologous promoter can be a ubiquitin promoter described in U.S. Pat. No. 6,528,701, which is incorporated by reference herein. Various tissue-specific promoters that can be used are described in U.S. Pat. Nos. 7,763,774 and 7,767,801, each of which is incorporated by reference herein.

Also provided is a DNA construct, preferably a vector, comprising any of the above nucleic acids or recombinant genes. The nucleic acid can comprise a nucleic acid comprising the nucleotide sequence of SEQ ID NO: 1 or 47. The nucleic acid can comprise a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47. Alternatively, the nucleic acid can hybridize, under stringent hybridization conditions, with the complementary strand of a nucleic acid comprising the nucleotide sequence of SEQ ID NO: 1 or 47 or a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47. In some embodiments, the DNA construct is a plasmid.

### Plant Cells

In another aspect is provided a plant cell comprising one or more of the booster polypeptide, nucleic acids, recombinant genes and DNA constructs described herein, preferably as transgene(s). In some embodiments, the booster polypeptide comprises the amino acid sequence of SEQ ID NO: 2 or 48. In some embodiments, the booster polypeptide comprises the amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 2 or 48. The nucleic acid can comprise a nucleic acid comprising the nucleotide sequence of SEQ ID NO: 1 or 47. The nucleic acid can comprise a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47. The nucleic acid can hybridize, under stringent hybridization conditions, with the complementary strand of a nucleic acid comprising the nucleotide sequence of SEQ ID NO: 1 or 47 or a nucleic acid comprising a nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1 or 47. Also provided is a plant, a part of the plant, a seed, an embryo or callus comprising the plant cell.

Plant cells can be part of or derived from any type of plant material, preferably shoot, hypocotyl, cotyledon, stem, leave, petiole, root, embryo, callus, flower, gametophyte or part thereof or can be a protoplast or derived from a protoplast. It is possible to use isolated plant cells as well as plant material, i.e. whole plants or parts of plants containing the plant cells.

A part of a plant, or parts of plants, may be attached to or separated from a whole intact plant. Such parts of a plant include, but are not limited to, organs, tissues, and cells of a plant, and preferably seeds.

The plant cell, plant part or plant can be from any plant species, whether monocot or dicot. Preferably, plants which may be subject to the methods and uses of the present invention are plants of the genus selected from the group consisting ofei Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, or Helianthus. More preferably, the plant is selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, Triticum aestivum, Hordeum vulgare, Secale cereale, Helianthus annuus, Solanum tuberosum, Sorghum bicolor, Brassica rapa, Brassica napus, Brassica juncacea, Brassica oleracea, Raphanus sativus, Oryza sativa, Glycine max, and/or Gossypium sp.

Genetically modified plant cells can be part of a whole plant or part thereof. Thus, the present invention also relates to a plant or plant part comprising the above genetically modified plant cell.

The plant cells into which the genome engineering components have been (co-) introduced are cultured under conditions allowing the genetic modification of the genome of said plant cell by integration of the transgene of interest and activity of the genome engineering components in the presence of the at least one boost factors.

### Genetic Modification of a Plant Cell

Also provided is a method for genetic modification in a plant cell. The method comprises introducing into the plant cell (i) any of the booster polypeptides, nucleic acids, recombinant genes or DNA constructs described herein; and (ii) a transgene and/or a genome engineering component. The plant cell may be cultivated under conditions allowing the synthesis of the booster polypeptide from the nucleic acid, the recombinant gene or the DNA construct. The plant cell may be cultivated under conditions allowing the genetic modification of the genome of said plant cell by activity of the genome engineering component in the presence of the booster polypeptide.

The genome engineering component can be introduced as a protein and/or as a nucleic acid encoding the genome engineering component, in particular as DNA such as plasmid DNA, RNA, mRNA or RNP. Genome engineering can be used for the manufacture of transgenic, gene-edited or base-edited plant material.

For plant cells to be modified, transformation methods based on biological approaches may be used, such as Agrobacterium transformation or viral vector-mediated plant transformation. A common biological means is transformation with Agrobacterium spp. which has been used for decades for a variety of different plant materials. Viral vector mediated plant transformation also can be used to introduce genetic material into a cell of interest. Agrobacterium-mediated transformation refers to the method of using Agrobacterium tumefaciens, a soil bacterium that works as a natural genetic engineer vector, to deliver foreign DNA into plant cells. Agrobacterium tumefaciens can invade plants and transfer foreign DNA in remarkably broad range of plants.

Alternatively, transformation methods based on physical delivery methods may be used, like particle bombardment or microinjection. Particle bombardment includes biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Particle bombardment and microinjection have evolved as prominent techniques for introducing genetic material into a plant cell or tissue of interest. Helenius et al., “Gene delivery into intact plants using the Helios™ Gene Gun”, Plant Molecular Biology Reporter, 2000, 18 (3):287-288 discloses a particle bombardment as physical method for introducing material into a plant cell. Thus, there exists a variety of plant transformation methods to introduce genetic material in the form of a genetic construct into a plant cell of interest, comprising biological and physical means known to the skilled person on the field of plant biotechnology and which can be applied to introduce at least one gene encoding at least one wall-associated kinase into at least one cell of at least one of a plant cell, tissue, organ, or whole plant.

The term “particle bombardment” as used herein, also named “biolistic transfection” or “microparticle-mediated gene transfer” refers to a physical delivery method for transferring a coated microparticle or nanoparticle comprising boost genes, booster polypeptides, genome engineering components, and/or transgenes into a target cell or tissue. The micro- or nanoparticle functions as projectile and is fired on the target structure of interest under high pressure using a suitable device, often called gene-gun. The transformation via particle bombardment uses a microprojectile of metal covered with the construct of interest, which is then shot onto the target cells using an equipment known as “gene gun” (Sandford et al. 1987) at high velocity fast enough (˜1500 km/h) to penetrate the cell wall of a target tissue, but not harsh enough to cause cell death. For protoplasts, which have their cell wall entirely removed, the conditions are different logically. The precipitated construct on the at least one microprojectile is released into the cell after bombardment. The acceleration of microprojectiles is accomplished by a high voltage electrical discharge or compressed gas (helium). Concerning the metal particles used it is mandatory that they are non-toxic, non-reactive, and that they have a lower diameter than the target cell. The most commonly used are gold or tungsten. There is plenty of information publicly available from the manufacturers and providers of gene-guns and associated system concerning their general use.

In a particularly preferred embodiment of microparticle bombardment, one or more boost genes, booster polypeptides, genome engineering components, and/or transgenes are co-delivered via microcarriers comprising gold particles having a size in a range of 0.4-1.6 micron (μm), preferably 0.4-1.0 μm. In an exemplary process, 10-1000 μg of gold particles, preferably 50-300 μg, are used per one bombardment.

The boost genes, booster polypeptides, genome engineering components, and/or transgenes can be delivered into target cells for example using a Bio-Rad PDS-1000/He particle gun or handheld Helios gene gun system. When a PDS-1000/He particle gun system used, the bombardment rupture pressures are from 450 psi to 2200 psi, preferred from 450-1100 psi, while the rupture pressures are from 100-600 psi for a Helios gene gun system. More than one chemical or construct can be co-delivered with genome engineering components into target cells simultaneously.

The above-described delivery methods for transformation and transfection can be applied to introduce the tools of the present invention simultaneously. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. The above delivery techniques, alone or in combination, can be used for in vivo (including in planta) or in vitro approaches.

In some embodiments, the genome engineering component comprises:
a) an enzyme inducing a double-stranded break (DSB) or a nucleic acid encoding same, and optionally a repair nucleic acid molecule, wherein the DSB-inducing enzyme optionally recognizes a predetermined site in the genome of said cell;
b) an enzyme inducing a single-stranded break (SSB) or a nucleic acid encoding same, and optionally a repair nucleic acid molecule, wherein the SSB-inducing enzyme optionally recognizes a predetermined site in the genome of said cell;
c) a base editor enzyme, optionally fused to a disarmed DSB- or SSB-inducing enzyme, wherein the base editor enzyme preferably recognizes a predetermined site in the genome of said cell; or
d) an enzyme effecting DNA methylation, histone acetylation, histone methylation, histone ubiquitination, histone phosphorylation, histone ribosylation or histone citrullination, optionally fused to a disarmed DSB- or SSB-inducing enzyme, wherein the enzyme preferably recognizes a predetermined site in the genome of said cell.

In order to enable a break at a predetermined target site, the enzymes preferably include a binding/recognition domain and a cleavage domain. Particular enzymes capable of inducing double or single-stranded breaks are nucleases or nickases as well as variants thereof, including such molecules no longer comprising a nuclease or nickase function but rather operating as recognition molecules in combination with another enzyme. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas9, Cpf1, Csm1, CasX or CasY nucleases as part of the Clustered Regularly Interspaced Short Palindromic Repeats (CRISPR) system. Thus, in a preferred aspect of the invention, the genome engineering component comprises a DSB- or SSB-inducing enzyme or a variant thereof selected from a CRISPR/Cas endonuclease, preferably a CRISPR/Cas9 endonuclease a CRISPR/Cpf1 endonuclease, or a CRISPR/Csm1 endonuclease, a zinc finger nuclease (ZFN), a homing endonuclease, a meganuclease and a TAL effector nuclease.

Rare-cleaving endonucleases are DSBI/SSBI enzymes that have a recognition site of preferably about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level. A list of other rare cleaving meganucleases and their respective recognition sites is provided in Table I of WO 03/004659 (pages 17 to 20) (incorporated herein by reference).

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO 94/18313 or WO 95/09233 and in Isalan et al. (2001). A rapid, generally applicable method to engineer zinc fingers illustrated by targeting the HIV-1 promoter. Nature biotechnology, 19(7): 656; Liu et al. (1997). Design of polydactyl zinc-finger proteins for unique addressing within complex genomes. Proceedings of the National Academy of Sciences, 94(11): 5525-5530.

Another example of custom-designed endonucleases includes the TALE nucleases (TALENs), which are based on transcription activator-like effectors (TALEs) from the bacterial genus Xanthomonas fused to the catalytic domain of a nuclease (e.g. FokI or a variant thereof). The DNA binding specificity of these TALEs is defined by repeat-variable di-residues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e.g. Boch et al. (2009). Breaking the code of DNA binding specificity of TAL-type III effectors. Science, 326(5959), 1509-1512; Moscou & Bogdanove (2009). A simple cipher governs DNA recognition by TAL effectors. Science, 326(5959), 1501-1501; and WO 2010/079430, WO 2011/072246, WO 2011/154393, WO 2011/146121, WO 2012/001527, WO 2012/093833, WO 2012/104729, WO 2012/138927, WO 2012/138939). WO 2012/138927 further describes monomeric (compact) TALENs and TALEs with various catalytic domains and combinations thereof.

Recently, a new type of customizable endonuclease system has been described; the so-called CRISPR/Cas system. A CRISPR system in its natural environment describes a molecular complex comprising at least one small and individual non-coding RNA in combination with a Cas nuclease or another CRISPR nuclease like a Cpf1 nuclease or a Csm1 nuclease (Zetsche et al., “Cpf1 Is a Single RNA-Guides Endonuclease of a Class 2 CRISPR-Cas System”, Cell, 163, pp. 1-13, October 2015.; US 2017/0233756 A1) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems code for a Cas9 nuclease as the key enzyme for the interference step, which system contains both a crRNA and also a trans-activating RNA (tracrRNA) as the guide motif. These hybridize and form double-stranded (ds) RNA regions which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease.

As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337:816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

The cleavage site of a DSBI/SSBI enzyme relates to the exact location on the DNA or RNA where the break is induced. The cleavage site may or may not be comprised in (overlap with) the recognition site of the DSBI/SSBI enzyme and hence it is said that the cleavage site of a DSBI/SSBI enzyme is located at or near its recognition site. The recognition site of a DSBI/SSBI enzyme, also sometimes referred to as binding site, is the nucleotide sequence that is (specifically) recognized by the DSBI/SSBI enzyme and determines its binding specificity. For example, a TALEN or ZNF monomer has a recognition site that is determined by their RVD repeats or ZF repeats respectively, whereas its cleavage site is determined by its nuclease domain (e.g. FokI) and is usually located outside the recognition site. In case of dimeric TALENs or ZFNs, the cleavage site is located between the two recognition/binding sites of the respective monomers, this intervening DNA or RNA region where cleavage occurs being referred to as the spacer region.

A person skilled in the art would be able to either choose a DSBI/SSBI enzyme recognizing a certain recognition site and inducing a DSB or SSB at a cleavage site at or in the vicinity of the preselected/predetermined site or engineer such a DSBI/SSBI enzyme. Alternatively, a DSBI/SSBI enzyme recognition site may be introduced into the target genome using any conventional transformation method or by crossing with an organism having a DSBI/SSBI enzyme recognition site in its genome, and any desired nucleic acid may afterwards be introduced at or in the vicinity of the cleavage site of that DSBI/SSBI enzyme.

In various embodiments, in modification of the genome comprises one or more of: i) a replacement of at least one nucleotide; ii) a deletion of at least one nucleotide; iii) an insertion of at least one nucleotide; iv) a change of the DNA methylation; and v) a change in histone acetylation, histone methylation, histone ubiquitination, histone phosphorylation, histone sumoylation, histone ribosylation or histone citrullination.

In some embodiments, the activity of the genome engineering component induces one or more double-stranded breaks in the genome of the plant cell, one or more single strand breaks in the genome of the plant cell, one or more base editing events in the genome of the plant cell, or one or more of DNA methylation, histone acetylation, histone methylation, histone ubiquitination, histone phosphorylation, histone sumoylation, histone ribosylation or histone citrullination in the genome of the plant cell.

In some embodiments, the induction of one or more double-stranded breaks or one or more single strand breaks is followed by non-homologous end joining (NHEJ) and/or by homology directed repair of the break(s) though a homologous recombination mechanism (HDR). NHEJ and HDR are two major and distinct pathways to repair breaks. Homologous recombination requires the presence of a homologous sequence as a template (e.g., repair nucleic acid molecule or “donor”) to guide the cellular repair process and the results of the repair are error-free and predictable. In the absence of a template (or repair nucleic acid molecule or “donor”) sequence for homologous recombination, the cell typically attempts to repair the break via the process of non-homologous end-joining (NHEJ).

In a particularly preferred aspect of this embodiment, a repair nucleic acid molecule is additionally introduced into the plant cell. The repair nucleic acid molecule is a single-stranded or double-stranded DNA molecule or RNA molecule that is used as a template for modification of the genomic DNA or the RNA at the preselected site in the vicinity of or at the cleavage site. In some embodiments, the repair nucleic acid molecule is used as a template for modification of the genomic DNA, in which the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region). Integration by homologous recombination allows for precise joining of the repair nucleic acid molecule to the target genome up to the nucleotide level, while NHEJ may result in small insertions/deletions at the junction between the repair nucleic acid molecule and genomic DNA.

In various embodiments of the aspects described herein, a modification of the genome occurs in which the genome has changed by at least one nucleotide. Modification of the genome can occur by insertion of a transgene, preferably an expression cassette comprising a transgene of interest, replacement of at least one nucleotide and/or a deletion of at least one nucleotide and/or an insertion of at least one nucleotide, as long as it results in a total change of at least one nucleotide compared to the nucleotide sequence of the preselected genomic target site before modification, thereby allowing the identification of the modification, e.g., by techniques such as sequencing or PCR analysis and the like, of which the skilled person will be well aware.

Modification of the genome may occur at a preselected site, a predetermined site, or predefined site, i.e., at a particular nucleotide sequence in the genome (e.g. the nuclear genome or the chloroplast genome) at which location it is desired to insert, replace and/or delete one or more nucleotides. For example, the preselected site, predetermined site, or predefined site can be an endogenous locus or a particular nucleotide sequence in or linked to a previously introduced foreign DNA, RNA or transgene. The preselected site can be a particular nucleotide position at (after) which it is intended to make an insertion of one or more nucleotides. The preselected site can also comprise a sequence of one or more nucleotides which are to be exchanged (replaced) or deleted.

In various embodiments, the length and percentage sequence identity of the flanking regions is chosen such as to enable homologous recombination between said flanking regions and their corresponding DNA region upstream or downstream of the preselected site. The DNA region or regions flanking the preselected site having homology to the flanking DNA region or regions of the repair nucleic acid molecule are also referred to as the homology region or regions in the genomic DNA.

To have sufficient homology for recombination, the flanking DNA regions of the repair nucleic acid molecule may vary in length, and should be at least about 10 nt, about 15 nt, about 20 nt, about 25 nt, about 30 nt, about 40 nt or about 50 nt in length. However, the flanking region may be as long as is practically possible (e.g. up to about 100-150 kb such as complete bacterial artificial chromosomes (BACs). Preferably, the flanking region will be about 50 nt to about 2000 nt, e.g. about 100 nt, 200 nt, 500 nt or 1000 nt. Moreover, the regions flanking the DNA of interest need not be identical to the homology regions (the DNA regions flanking the preselected site) and may have between about 80% to about 100% sequence identity, preferably about 95% to about 100% sequence identity with the DNA regions flanking the preselected site. The longer the flanking region, the less stringent the requirement for homology. Furthermore, to achieve exchange of the target DNA sequence at the preselected site without changing the DNA sequence of the adjacent DNA sequences, the flanking DNA sequences should preferably be identical to the upstream and downstream DNA regions flanking the preselected site.

In order to target sequence modification at the preselected site, the flanking regions must be chosen so that 3′ end of the upstream flanking region and/or the 5′ end of the downstream flanking region align(s) with the ends of the predefined site. As such, the 3′ end of the upstream flanking region determines the 5′ end of the predefined site, while the 5′ end of the downstream flanking region determines the 3′ end of the predefined site.

The preselected site is located outside or away from said cleavage (and/or recognition) site, such that the site where it is intended to make the genomic modification (the preselected site) does not comprise the cleavage site and/or recognition site of the DSBI/SSBI enzyme, such that the preselected site does not overlap with the cleavage (and/or recognition) site. Outside/away from in this respect thus means upstream or downstream of the cleavage (and/or recognition) site.

In various embodiments, the at least one base editor according to the present invention is temporarily or permanently linked to at least one site-specific DSBI/SSBI enzyme complex or at least one modified site-specific DSBI/SSBI enzyme complex, or optionally to a component of said at least one site-specific DSBI/SSBI enzyme complex. The linkage can be covalent and/or non-covalent. Any base editor or site-specific DSBI/SSBI enzyme complex, or a catalytically active fragment thereof, or any component of a base editor complex or of a site-specific DSBI/SSBI enzyme complex as disclosed herein can be introduced into a cell as a nucleic acid fragment, the nucleic acid fragment representing or encoding a DNA, RNA or protein effector, or it can be introduced as DNA, RNA and/or protein, or any combination thereof.

The base editor is a protein or a fragment thereof having the capacity to mediate a targeted base modification, i.e., the conversion of a base of interest resulting in a point mutation of interest. Preferably, the at least one base editor in the context of the present invention is temporarily or permanently fused to at least one DSBI/SSBI enzyme, or optionally to a component of at least one DSBI/SSBI. The fusion can be covalent and/or non-covalent. Multiple publications have shown targeted base conversion, primarily cytidine (C) to thymine (T), using a CRISPR/Cas9 nickase or non-functional nuclease linked to a cytidine deaminase domain, Apolipoprotein B mRNA-editing catalytic polypeptide (APOBEC1), e.g., APOBEC derived from rat. The deamination of cytosine (C) is catalyzed by cytidine deaminases and results in uracil (U), which has the base-pairing properties of thymine (T). Most known cytidine deaminases operate on RNA, and the few examples that are known to accept DNA require single-stranded (ss) DNA. Studies on the dCas9-target DNA complex reveal that at least nine nucleotides (nt) of the displaced DNA strand are unpaired upon formation of the Cas9-guide RNA-DNA ‘R-loop’ complex (Jore et al., Nat. Struct. Mol. Biol., 18, 529-536 (2011)). Indeed, in the structure of the Cas9 R-loop complex, the first 11 nt of the protospacer on the displaced DNA strand are disordered, suggesting that their movement is not highly restricted. It has also been speculated that Cas9 nickase-induced mutations at cytosines in the non-template strand might arise from their accessibility by cellular cytosine deaminase enzymes. It was reasoned that a subset of this stretch of ssDNA in the R-loop might serve as an efficient substrate for a dCas9-tethered cytidine deaminase to effect direct, programmable conversion of C to U in DNA (Komor et al., supra). Recently, Goudelli et al., Programmable base editing of A⋅T to G⋅C in genomic DNA without DNA cleavage, Nature, 2017, 551(7681), 464, described adenine base editors (ABEs) that mediate the conversion of A⋅T to G⋅C in genomic DNA.

Enzymes effecting DNA methylation, as well as histone-modifying enzymes have been identified in the art. Histone posttranslational modifications play significant roles in regulating chromatin structure and gene expression. For example, enzymes for histone acetylation are described in Sterner D. E., Berger S. L. (June 2000): “Acetylation of histones and transcription-related factors”, Microbiol. Mol. Biol. Rev. 64 (2): 435-59. Enzymes effecting histone methylation are described in Zhang Y., Reinberg D (2001): “Transcription regulation by histone methylation: interplay between different covalent modifications of the core histone tails”, Genes Dev. 15 (18): 2343-60. Histone ubiquitination is described in Shilatifard A (2006): “Chromatin modifications by methylation and ubiquitination: implications in the regulation of gene expression”, Annu. Rev. Biochem. 75:243-69. Enzymes for histone phosphorylation are described in Nowak S. J., Corces V. G. (April 2004): “Phosphorylation of histone H3: a balancing act between chromosome condensation and transcriptional activation”, Trends Genet. 20 (4): 214-20. Enzymes for histone sumoylation are described in Nathan D., Ingvarsdottir K., Sterner D. E., et al. (April 2006): “Histone sumoylation is a negative regulator in Saccharomyces cerevisiae and shows dynamic interplay with positive-acting histone modifications”, Genes Dev. 20 (8): 966-76. Enzymes for histone ribosylation are described in Hassa P. O., Haenni S. S., Elser M., Hottiger M. O. (September 2006): “Nuclear ADP-ribosylation reactions in mammalian cells: where are we today and where are we going?”, Microbiol. Mol. Biol. Rev. 70 (3): 789-829. Histone citrullination is catalyzed for example by an enzyme called peptidylarginine deiminase 4 (PAD4, also called PADI4), which converts both histone arginine (Arg) and mono-methyl arginine residues to citrulline.

Enzymes effecting DNA methylation and histone-modifying enzymes may be fused to a disarmed DSB or SSB inducing enzyme, which preferably recognizes a predetermined site in the genome of said cell.

### Exemplary Transgenes

In various embodiments of the methods for genetic modification in a plant cell, the transgene may be a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

In various embodiments of the methods for genetic modification in a plant cell, the method is effective to promote cell proliferation or cell regeneration, or is effective to increase the efficiency for regeneration of transgenic, gene edited or base edited plants The method is effective preferably after genetic modification/modification of the genome. In various embodiments of the methods for genetic modification in a plant cell, the method is effective to induce direct or indirect (somatic) embryogenesis from a single cell, preferably an embryonic cell, a somatic cell or a protoplast, or from a callus cell, or from a callus cell. The method is effective preferably after genetic modification/modification of the genome. In various embodiments, the method is effective to increase the stable transformation efficiency of the transgene into the plant cell or is effective to increase the efficiency for generation of transgenic plants. In various embodiments, the method is effective to increase the efficiency of the genome engineering component to edit the genome of the plant cell or is effective to increase the efficiency for generation of transgenic, gene edited or base edited plants.

In some embodiments, the method is effective to improve the efficiency of regeneration of plants derived from recalcitrant genotypes, is effective to improve the efficiency of regeneration of plants from non-conventional tissue types, or is effective to accelerate the regeneration process, preferably after genetic modification/modification of the genome.

### Transient Expression of Booster Polypeptide and Boost Genes

Also provided is a method for transient expression of a booster polypeptide and/or a boost gene in a plant cell. The method comprises introducing into the plant cell (i) a booster polypeptide, nucleic acid, recombinant gene or DNA construct described herein; and (ii) a transgene and/or a genome engineering component.

In some embodiments, one or more of the booster polypeptide and boost genes are transiently co-expressed. The co-expression may be effective to promote cell proliferation. Such co-expression may be effective to promote cell regeneration. The co-expression may be effective to induce embryogenesis from single cells, and thus provide ability to regenerate homogenous plants without selection. The co-expression may improve genome editing efficiency by co-delivery with genome-editing components. Co-expression may comprise transiently co-introducing a boost polypeptide (e.g., KWS-RBP-1) with one or more nucleic acids encoding a boost gene (e.g., PLT5, PLT7, RKD4, and RKD2).

Transient co-delivery of booster polypeptides and/or one or more boost genes may be carried out as described in U.S. Provisional Application No. 62/685,626, incorporated by reference herein in its entirety.

In various embodiments, other boost factors such as chemical HDACi and phytohormones can be delivered, as described in U.S. Provisional Application No. 62/685,626.

In some embodiments, the booster polypeptide is transiently present, transiently active and/or transiently expressed in the plant cell. In some embodiments, the nucleic acid encoding the booster polypeptide is transiently present, transiently active and/or transiently expressed in the plant cell. One or more polypeptides selected from the group consisting of a PLT5 polypeptide, a PLT7 polypeptide, and/or one or more nucleic acids selected from the group consisting of a nucleic acid encoding a PLT5 polypeptide, a PLT7 polypeptide, and an RKD2 polypeptide, and/or one or more site-directed transcriptional activators suitable to increase transiently the expression of an endogenous PLT5 polypeptide, an endogenous PLT7 polypeptide, or an endogenous RKD2 polypeptide, and/or a nucleic acid encoding such site-directed transcriptional activator can also be introduced into the plant cell.

Transient expression can be carried out by transient transformation/transfection of a boost protein/polypeptide or nucleic acid fragment encoding the protein/polypeptide, expressed preferably under a strong constitutive promoter. Transient expression of a nucleic acid encoding a PLT5 polypeptide, a nucleic acid encoding a PLT7 polypeptide, and/or one or more site-directed transcriptional activators suitable to increase transiently the expression of an endogenous PLT5 polypeptide, an endogenous PLT7 polypeptide, can also be realized by stable transformation of a boost gene under the control of a tissue and development specific promoter or an inducible promoter. The boost genes can be expressed and then be active transiently. The boost genes can then be turned off and degraded shortly when plant cell development is changed or the inducing condition(s) are removed. For example, the strong constitutive promoter from Brachypodium EF1 gene, pBdEF1 (SEQ ID NO: 23) may be used to drive a boost gene for transient transformation (see, e.g., Example 1).

Transient expression can arise from any of transient transfection, transient transformation, and stable transformation. “Transient transformation” and “transient transfection” comprise the transfer of a foreign material [i.e. a nucleic acid fragment, protein, ribonucleoprotein (RNP), etc.] into host cells resulting in gene expression and/or activity without integration and stable inheritance of the foreign material. The foreign components are not permanently incorporated into the cellular genome, but provide a temporal action resulting in a modification of the genome. A transient transformation event may be unable to be transmitted to next generation, and thus is non-inheritable. “Stable transformation” refers to the event where a transferred nucleic acid fragment is integrated into the genome of a host cell (includes both nuclear and organelle genomes) resulting to stable inheritance of the nucleic acid fragment.

For example, transient expression can be used for transient genome editing. Transient activity and/or transient presence of the genome engineering component in the plant cell can result in introduction of one or more double-stranded breaks in the genome of the plant cell, one or more single-stranded breaks in the genome of the plant cell, one or more base-editing events in the genome of the plant cell, or one or more of DNA methylation, histone acetylation, histone methylation, histone ubiquitination, histone phosphorylation, histone sumoylation, histone ribosylation or histone citrullination in the genome of the plant cell. The resulting modification in the genome of the plant cell can, for example, be selected from a replacement of at least one nucleotide, a deletion of at least one nucleotide, an insertion of at least one nucleotide, a change of DNA methylation, a change in histone acetylation, histone methylation, histone ubiquitination, histone phosphorylation, histone sumoylation, histone ribosylation, or histone citrullination or any combination thereof.

The site-directed transcriptional activator means a synthetic transcription factor described in U.S. Provisional Application No. 62/609,508, incorporated by reference herein. The synthetic transcription factor can comprise at least one recognition domain and at least one gene expression modulation domain, in particular an activation domain, wherein the synthetic transcription factor is configured to modulate the expression of an endogenous gene in the genome of plant or plant cell. Such an endogenous gene is preferably a (native) morphogenic gene which encodes polypeptides involved in plant developmental processes like root formation or shoot formation. In some embodiments, the endogenous morphogenic gene is selected from the group consisting of an endogenous nucleic acid encoding a PLT5 polypeptide, an endogenous nucleic acid encoding a PLT7 polypeptide, an endogenous nucleic acid encoding an RKD4 polypeptide, or an endogenous nucleic acid encoding an RKD2polypeptide. In some embodiments, the at least one recognition domain is, or is a fragment of, a molecule selected from the group consisting of at least one TAL effector, at least one disarmed CRISPR/nuclease system, at least one Zinc-finger domain, and at least one disarmed homing endonuclease, or any combination thereof.

In some embodiments, the at least one disarmed CRISPR/nuclease system is a CRISPR/dCas9 system, a CRISPR/dCpf1 system, a CRISPR/dCsm1 system, a CRISPR/dCasX system or a CRISPR/dCasY system, or any combination thereof, and wherein the at least one disarmed CRISPR/nuclease system comprises at least one guide RNA.

In some embodiments, the at least one activation domain is selected from the group consisting of an acidic transcriptional activation domain, preferably, wherein the at least one activation domain is from a TAL effector gene of Xanthomonas oryzae, VP16 or tetrameric VP64 from Herpes simplex, VPR, SAM, Scaffold, Suntag, P300, VP160, or any combination thereof. In some embodiments, the activation domain is VP64.

In some embodiments, the synthetic transcription factor is configured to modulate expression, preferably transcription, of the morphogenic gene by binding to a regulation region located at a certain distance in relation to the start codon. In preferred embodiments, the synthetic transcription factor is configured to increase expression, preferably transcription, of the morphogenic gene by binding to a regulation region located at a certain distance in relation to the start codon.

In some embodiments, the site-directed transcriptional activator/synthetic transcription factor, or the nucleic acid encoding the same, comprises at least one recognition domain and at least one activation domain, wherein the site-directed transcriptional activator is configured to increase the expression of an endogenous PLT5 polypeptide, an endogenous PLT7 polypeptide, an endogenous RKD4 polypeptide, or an endogenous RKD2 polypeptide, preferably by binding to a regulation region located at a certain distance in relation to the start codon of the endogenous PLT5 polypeptide, the endogenous PLT7 polypeptide, the endogenous RKD4 polypeptide, or the endogenous RKD2 polypeptide.

The “regulation region” as used herein refer to the binding site of at least one recognition domain to a target sequence in the genome at or near a morphogenic gene. There may be two discrete regulation regions, or there may be overlapping regulation regions, depending on the nature of the at least one activation domain and the at least one recognition domain as further disclosed herein, which different domains of the synthetic transcription factor can be assembled in a modular manner.

In certain embodiments, the at least one recognition domain may target at least one sequence (recognition site) relative to the start codon of a gene of interest, which sequence may be at least 1.000 bp upstream (−) or downstream (+), −700 bp to +700 bp, −550 bp to +500 bp, or −550 bp to +425 bp relative to of the start codon of a gene of interest. Promoter-near recognizing recognition domains might be preferable in certain embodiments, whereas it represents an advantage of the specific synthetic transcription factors that the targeting range of the synthetic transcription factors is highly expanded over conventional or naturally occurring transcription factors. As the recognition and/or the activation domains can be specifically designed and constructed to specifically identify and target hot-spots of modulation.

In certain embodiments, the at least one recognition site may be −169 bp to −4 bp, −101 bp to −48 bp, −104 to −42 bp, or −175 to +450 bp (upstream (−) or downstream (+), respectively) relative to the start codon of a gene of interest to provide an optimum sterical binding environment allowing the best modulation, preferably transcriptional activation, activity. In particular for CRISPR-based synthetic transcription factors acting together with a guide RNA as recognition moiety, the binding site can also reside in within the coding region of a gene of interest (downstream of the start codon of a gene of interest).

In further embodiments, the recognition domain of the synthetic transcription factor can bind to the 5′ and/or 3′ untranslated region (UTR) of a gene of interest. In embodiments, where different recognition domains are employed, the at least two recognition domains can bind to different target regions of a morphogenic gene, including 5′ and/or 3′UTRs, but they can also bind outside the gene region, but still in a certain distance of at most 1 to 1.500 bps thereto. One preferred region, where a recognition domain can bind, resides about −4 bp to about −300, preferably about −40 bp to about −170 bp upstream of the start codon of a morphogenic gene of interest. Furthermore, the length of a recognition domain and thus the corresponding recognition site in a genome of interest may thus vary depending on the synthetic transcription factor and the nature of the recognition domain applied. Based on the molecular characteristics of the at least one recognition domain, this will also determine the length of the corresponding at least one recognition site. For example, where individual zinc finger may be from about 8 bp to about 20 bp, wherein arrays of between three to six zinc finger motifs may be preferred, individual TALE recognition sites may be from about 11 to about 30 bp, or more. Recognition sites of gRNAs of a CRISPR-based synthetic transcription factor comprise the targeting or “spacer” sequence of a gRNA hybridizing to a genomic region of interest, whereas the gRNA comprises further domains, including a domain interacting with a disarmed CRISPR effector. The recognition site of a synthetic transcription factor based on a disarmed CRISPR effector will comprise a PAM motif, as the PAM sequence is necessary for target binding of any CRISPR effector and the exact sequence is dependent upon the species of the CRISPR effector, i.e., a disarmed CRISPR effector.

### Introduction of Boost Genes and Boost Polypeptides

The boosters and/or genome engineering components can be introduced as a protein/polypeptide or as a nucleic acid encoding the protein/polypeptide, in particular as protein/polypeptide, or DNA such as plasmid DNA, RNA, mRNA or RNP.

The boosters may be co-delivered with one or more genome engineering components. As used herein, “co-delivery” or “co-deliver” and “co-introduction” or “co-introduce” are used interchangeably. In terms of the present invention, “co-introducing” refers to the process, in which at least two different components are delivered into the same plant cell concurrently. Thus, the genome engineering components and boost factors are introduced together into the same plant cell. Preferably, both types of components, booster and genes of interest, are introduced via separate constructs. Co-introduction into the plant cell can be conducted by particle bombardment, microinjection, Agrobacterium-mediated transformation, electroporation, electrofusion, agroinfiltration or vacuum infiltration.

### Regeneration Boost Genes

It is believed that transformed cells are less regenerable than wild type cells. Transformed cells are susceptible to programmed cell death due to presence of foreign DNA inside of the cells. Stresses arising from delivery (e.g. bombardment damage) may trigger a cell death as well. Therefore, promoting cell division is essential for the regeneration of the modified cells. Further, genome engineering efficiency is controlled largely by host cell statuses. Cells undergoing rapid cell-division, like those in plant meristem, are the most suitable recipients for genome engineering. Promoting cell division will probably increase DNA integration or modification during DNA replication and division process, and thus increase genome engineering efficiency.

The boost genes and booster polypeptides according to the invention, KWS-RBP1 (SEQ ID NO: 2) and KWS-RBP2 (SEQ ID NO: 48) are man-made and have been designed to improve the activity of the genome engineering component. When a booster polypeptide is introduced into a plant cell along with a transgene, the booster polypeptide can increase expression of the transgene and polypeptides encoded by the transgene. When the booster polypeptide is introduced into a plant cell along with a genome engineering component and the transgene, the activity of the genome engineering component may be increased. Such increase may result in more efficient integration of the transgene into the genome of the plant cell. One or more boost genes can be co-expressed with the booster polypeptide. One or more boost genes can be co-transfected with the booster polypeptide.

Such additional boost genes are selected based on their functions involved in promoting cell division and plant morphogenesis. Each of the candidate genes are cloned and driven by a strong constitutive promoter, and evaluated by transient expression in corn cells without a selection. Examples for boost genes are PLT5 (PLETHORA5; SEQ ID NOs: 4 and 6), PLT7 (PLETHORA7; SEQ ID NOs: 8, 10) and RKD2 (SEQ ID NOs: 18, 20 and 22).

PLT (PLETHORA), also called AIL (AINTEGUMENT-LIKE) genes, are members of the AP2 family of transcriptional regulators. Members of the AP2 family of transcription factors play important roles in cell proliferation and embryogenesis in plants (El Ouakfaoui, S., Schnell, J., Abdeen, A., Colville, A., Labbé, H., Han, S., Baum, B., Laberge, S., Miki, B (2010) Control of somatic embryogenesis and embryo development by AP2 transcription factors. PLANT MOLECULAR BIOLOGY 74(4-5): 313-326.). PLT genes are expressed mainly in developing tissues of shoots and roots, and are required for stem cell homeostasis, cell division and regeneration, and for patterning of organ primordia.

PLT family comprises an AP2 subclade of six members. Four PLT members, PLT1/AIL3 PLT2/AIL4, PLT3/AIL6, and BBM/PLT4/AIL2, are expressed partly overlap in root apical meristem (RAM) and required for the expression of QC (quiescent center) markers at the correct position within the stem cell niche. These genes function redundantly to maintain cell division and prevent cell differentiation in root apical meristem.

Three PLT genes, PLT3/AIL6, PLT5/AIL5, and PLT7/AIL7, are expressed in shoot apical meristem (SAM), where they function redundantly in the positioning and outgrowth of lateral organs. PLT3, PLT5, and PLT7, regulate de novo shoot regeneration in Arabidopsis by controlling two distinct developmental events. PLT3, PLT5, and PLT7 required to maintain high levels of PIN1 expression at the periphery of the meristem and modulate local auxin production in the central region of the SAM which underlies phyllotactic transitions. Cumulative loss of function of these three genes causes the intermediate cell mass, callus, to be incompetent to form shoot progenitors, whereas induction of PLT5 or PLT7 can render shoot regeneration in a hormone-independent manner. PLT3, PLT5, PLT7 regulate and require the shoot-promoting factor CUP-SHAPED COTYLEDON2 (CUC2) to complete the shoot-formation program. PLT3, PLT5, and PLT7, are also expressed in lateral root founder cells, where they redundantly activate the expression of PLT1 and PLT2, and consequently regulate lateral root formation.

The additional boost genes can be from any number of plants known in the art. Such plants include, but are not limited to, Zea mays, Arabidopsis thaliana, and Triticum aestivum. In some embodiments, the boost gene is Zea mays PLT5. In some embodiments, the boost gene is Arabidopsis thaliana PLT5. In some embodiments, the boost gene is Zea mays PLT7. In some embodiments, the boost gene is Arabidopsis thaliana PLT7. In some embodiments, the boost gene is Triticum aestivum RKD4. In some embodiments, the boost gene is Arabidopsis thaliana RKD4. In some embodiments, the boost gene is Zea mays RKD4. In some embodiments, the boost gene is Triticum aestivum RKD2. In some embodiments, the boost gene is Arabidopsis thaliana RKD2. In some embodiments, the boost gene is Zea mays RKD2.

In some embodiments, both the booster polypeptide according to the invention and the PLT5 polypeptide (encoded by the PLT5 boost gene) are introduced into the plant cell, and optionally transiently co-expressed. In some embodiments, both the booster polypeptide according to the invention and the PLT7 polypeptide (encoded by the PLT7 boost gene) are introduced into the plant cell, and optionally transiently co-expressed.

The polypeptide encoded by the PLT5 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4. The polypeptide encoded by the PLT5 boost gene may comprise the sequence of SEQ ID NO: 4. The polypeptide encoded by the PLT5 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 6. The polypeptide encoded by the PLT5 boost gene may comprise the sequence of SEQ ID NO: 6.

The polypeptide encoded by the Zea mays PLT5 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4. The polypeptide encoded by the Zea mays PLT5 boost gene may comprise the sequence of SEQ ID NO: 4.

The polypeptide encoded by the Arabidopsis thaliana PLT5 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 6. The polypeptide encoded by the Arabidopsis thaliana PLT5 boost gene may comprise the sequence of SEQ ID NO: 6.

The polypeptide encoded by the PLT7 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 8. The polypeptide encoded by the PLT7 boost gene may comprise the sequence of SEQ ID NO: 8. The PLT7 polypeptide may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 10. The polypeptide encoded by the PLT7 boost gene may comprise the sequence of SEQ ID NO: 10.

The polypeptide encoded by the Zea mays PLT7 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 8. The polypeptide encoded by the Zea mays PLT7 boost gene may comprise the sequence of SEQ ID NO: 8.

The polypeptide encoded by the Arabidopsis thaliana PLT7 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 10. The polypeptide encoded by the Arabidopsis thaliana PLT7 boost gene may comprise the sequence of SEQ ID NO: 10.

The polypeptide encoded by the RKD4 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 12. The polypeptide encoded by the RKD4 boost gene may comprise the sequence of SEQ ID NO: 12. The polypeptide encoded by the RKD4 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 14. The polypeptide encoded by the RKD4 boost gene may comprise the sequence of SEQ ID NO: 14. The polypeptide encoded by the RKD4 boost gene may comprise an amino acid sequence at least 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 16. The polypeptide encoded by the RKD4 boost gene may comprise the sequence of SEQ ID NO: 16.

The polypeptide encoded by the Triticum aestivum RKD4 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 12. The polypeptide encoded by the Triticum aestivum RKD4 boost gene may comprise the sequence of SEQ ID NO: 12.

The polypeptide encoded by the Arabidopsis thaliana RKD4 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 14. The polypeptide encoded by the Arabidopsis thaliana RKD4 boost gene may comprise the sequence of SEQ ID NO: 14.

The polypeptide encoded by the Zea mays RKD4 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 16. The polypeptide encoded by the Zea mays RKD4 boost gene may comprise the sequence of SEQ ID NO: 16.

The polypeptide encoded by the RKD2 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 18. The polypeptide encoded by the RKD2 boost gene may comprise the sequence of SEQ ID NO: 18.The polypeptide encoded by the RKD2 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 20. The polypeptide encoded by the RKD2 boost gene may comprise the sequence of SEQ ID NO: 20. The polypeptide encoded by the RKD2 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 22. The polypeptide encoded by the RKD2 boost gene may comprise the sequence of SEQ ID NO: 22.

The polypeptide encoded by the Triticum aestivum RKD2 boost gene may comprise an amino acid sequence at least 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 18. The polypeptide encoded by the Triticum aestivum RKD2 boost gene may comprise the sequence of SEQ ID NO: 18.

The polypeptide encoded by the Arabidopsis thaliana RKD2 boost gene may comprise an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 20. The polypeptide encoded by the Arabidopsis thaliana RKD2 boost gene may comprise the sequence of SEQ ID NO: 20.

The polypeptide encoded by the Zea mays RKD2 boost gene may comprise an amino acid sequence at least 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 22. The polypeptide encoded by the Zea mays RKD2 boost gene may comprise the sequence of SEQ ID NO: 22.

In some embodiments, the nucleic acid encoding the PLT5 polypeptide comprises a nucleic acid comprising the nucleotide sequence of SEQ ID NO: 3 or 5. In some embodiments, the nucleic acid encoding the PLT5 polypeptide comprises a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 3 or 5. In some embodiments, the nucleic acid encoding the PLT5 polypeptide comprises a nucleic acid hybridizing with the complementary strand of a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 3 or 5, or a nucleic acid comprising the nucleotide sequence of SEQ ID NO: 3 or 5.

In some embodiments, the nucleic acid encoding the PLT7 polypeptide comprises a nucleic acid comprising the nucleotide sequence of SEQ ID NO: 7 or 9. In some embodiments, the nucleic acid encoding the PLT7 polypeptide comprises a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7 or 9. In some embodiments, the nucleic acid encoding the PLT7 polypeptide comprises a nucleic acid hybridizing with the complementary strand of a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7 or 9, or a nucleic acid comprising the nucleotide sequence of SEQ ID NO: 7 or 9.

In some embodiments, the nucleic acid encoding the RKD4 polypeptide comprises a nucleic acid comprising the nucleotide sequence of SEQ ID NO: 11, 13, or 15. In some embodiments, the nucleic acid encoding the RKD4 polypeptide comprises a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 11, 13, or 15. In some embodiments, the nucleic acid encoding the RKD4 polypeptide comprises a nucleic acid hybridizing with the complementary strand of a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 11, 13, or 15, or a nucleic acid comprising the nucleotide sequence of SEQ ID NO: 11, 13, or 15.

In some embodiments, the nucleic acid encoding the RKD2 polypeptide comprises a nucleic acid comprising the nucleotide sequence of SEQ ID NO: 17, 19, or 21. In some embodiments, the nucleic acid encoding the RKD2 polypeptide comprises a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 17, 19, or 21. In some embodiments, the nucleic acid encoding the RKD2polypeptide comprises a nucleic acid hybridizing with the complementary strand of a nucleic acid comprising the nucleotide sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 17, 19, or 21, or a nucleic acid comprising the nucleotide sequence of SEQ ID NO: 17, 19, or 21.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as program NEEDLE as implemented in the European Molecular Biology Open Software Suite (EMBOSS), e.g. version 6.3.1.2 (Trends in Genetics 16 (6), 276 (2000)), with its default parameter, e.g. for proteins matrix=EBLOSUM62, gapopen=10.0 and gapextend=0.5.

As used herein, the term “hybridize(s)(ing)” refers to the formation of a hybrid between two nucleic acid molecules via base-pairing of complementary nucleotides. The term “hybridize(s)(ing) under stringent conditions” means hybridization under specific conditions. An example of such conditions includes conditions under which a substantially complementary strand, namely a strand composed of a nucleotide sequence having at least 80% complementarity, hybridizes to a given strand, while a less complementary strand does not hybridize. Alternatively, such conditions refer to specific hybridizing conditions of sodium salt concentration, temperature and washing conditions. As an example, highly stringent conditions comprise incubation at 42° C., 50% formamide, 5×SSC (150 mM NaCl, 15 mM trisodium citrate), 50 mM sodium phosphate, 5× Denhardt's solution, 10× dextran sulfate, 20 mg/ml sheared salmon sperm DNA and washing in 0.2×SSC at about 65° C. (SSC stands for 0.15 M sodium chloride and 0.015 M trisodium citrate buffer). Alternatively, highly stringent conditions may mean hybridization at 68° C. in 0.25 M sodium phosphate, pH 7.2, 7% SDDS, 1 mM EDTA and 1% BSA for 16 hours and washing twice with 2×SSC and 0.1% SDDS at 68° C. Further alternatively, highly stringent hybridisation conditions are, for example: Hybridizing in 4×SSC at 65° C. and then multiple washing in 0.1×SSC at 65° C. for a total of approximately 1 hour, or hybridizing at 68° C. in 0.25 M sodium phosphate, pH 7.2, 7% SDS, 1 mM EDTA and 1% BSA for 16 hours and subsequent washing twice with 2×SSC and 0.1% SDS at 68° C.

### Epigenetically-Regulating Chemicals

An epigenetically regulating chemical, e.g., protein deacetylase inhibitor (ii.1), can be co-introduced with the genome engineering component. Exemplary epigenetically regulating chemicals for use according to the invention include, but are not limited to, histone deacetylase inhibitors (HDACis) such as trichostatin A (TSA), and DNA methyltransferase inhibitors.

It is assumed that the co-delivered epigenetically regulating chemicals (ii.1) (in particular HDACis) relax plant chromatin structure, promote the DNA accessibility to the genome engineering components in the bombarded cells, thus consequently promote genome engineering (i.e. transformation and genome editing) efficiencies. The reason for this assumption is: The basic structural and functional unit of genetic material is the nucleosome, in which negatively charged DNA is wrapped around a positively charged histone octamer and associated linker histones. Nucleosome units further fold and pack into chromatin (Andrews, A. J., and Luger, K. (2011). Nucleosome structure(s) and stability: Variations on a theme. Annu. Rev. Biophys. 40:99-117.). DNA accessibility largely depends on compactness of the nucleosomes and chromatins. Chromatin-remodeling enzymes dynamically modify lysine or other amino acids of histones, which cause changes in their charges and interactions with DNA and other proteins, and result in chromatin folding or unfolding (Bannister A. J., Kouzarides T. (2011) Regulation of chromatin by histone modifications. Cell Res 21:381-95.). By adding or removing an acetyl group, acetylation and deacetylation of the lysine residue in histone proteins are often involved in the reversible modulation of chromatin structure in eukaryotes, and mediate chromatin accessibility and the regulation of gene expression. Histone deacetylases (HDAC) are enzymes that remove acetyl groups from lysine resides on the N-terminal tail of histones, which makes the histone more positively charged, and therefore allows the histone wrap DNA more tightly. Inhibition of HDACs might help chromatin unfolding and enable the DNA to be more accessible.

Chromatin remodeling and other epigenetic modifications surely play an important role in regulating cell totipotency and regeneration (Zhang, H., and Ogas, J. (2009). An epigenetic perspective on developmental regulation of seed genes. Mol. Plant 2:610-627.). Inhibition of histone deacetylase (HDAC) activities have been shown associated with plant regeneration and microspore embryogenesis (Miguel, C., and Marum, L., 2011. An epigenetic view of plant cells cultured in vitro: somaclonal variation and beyond. J. Exp. Bot. 62:3713-3725., Li Hui et al. (2014) The Histone Deacetylase Inhibitor Trichostatin A Promotes Totipotency in the Male Gametophyte Plant Cell, 26:195-209.). Inhibition of HDAC activity or downstream HDAC-mediated pathways plays a major role in the initiation of stress-induced haploid embryogenesis. One such HDACi is trichostatin A (TSA). It has been shown that TSA induces massive embryogenic cell proliferation in the male gametophyte of B. napus. TSA treatment leads to a high frequency of sporophytic cell division in cultured microspores and pollen.

Various methods may be used to increase further the genome engineering efficiency in presence of one or more epigenetically regulating chemicals, e.g. protein deacetylase inhibitors, in particular HDACi. Such an HDACi may be trichostatin A (TSA), N-Hydroxy-7-(4-dimethylaminobenzoyl)-aminoheptanamide (M344), suberoylanilide hydroxamic acid (SAHA), or others. These HDACis are selected from hydroxamic acid (HA)-based chemicals, which target to zinc dependent HDACs.

### Phytohormones

In various embodiments, one or more phytohormones, such as auxins and cytokinins like 2,4-D, 6-Benzylaminopurine (6-BA) and Zeatin, are co-delivered with one or more of a boost gene, a booster polypeptide, a genome engineering component, and a transgene.

Plant somatic cells are capable to resume cell division and regenerate into an entire plant in in-vitro culture through somatic embryogenesis or organogenesis, which largely depends on phytohormones, such as auxins and cytokinins. In the present invention it was found, that phytohormones promote cell proliferation, increase the sensitivity of the plant cells to genome engineering, and thus improve genome engineering (i.e. transformation and genome editing) efficiency.

One of auxins is 2,4-Dichlorophenoxyacetic acid (2,4-D), which is nearly indispensable for somatic embryogenesis and cell regeneration in monocot plants, e.g. maize and wheat. Meanwhile, cytokinins e.g. 6 benzylaminopurine (6-BA) or Zeatin, are essential for plant organogenesis, and shoot meristem initiation and development. The methods to improve genome engineering efficiency may include co-delivery of one or more of phytohormones (2,4-D, 6-BA, Zeatin, etc.) with the genome engineering component.

A genome engineering component and at least one of the epigenetically-regulating chemicals and phytohormones can be co-introduced into one plant cell.

As used herein, “co-delivery” or “co-deliver” and “co-introduction” or “co-introduce” are used interchangeably. In terms of the present invention, “co-introducing” refers to the process, in which at least two different components are delivered into the same plant cell concurrently. Thus, the genome engineering component and at least one of the epigenetically-regulating chemicals and phytohormones may be introduced together into the same plant cell.

Co-introduction into the plant cell can be conducted by particle bombardment, microinjection, Agrobacterium-mediated transformation, electroporation, agroinfiltration or vacuum infiltration. According to the invention, methods based on physical delivery like particle bombardment, microinjection, electroporation, nanoparticles, and cell-penetrating peptides (CPPs) are particularly preferred for co-introducing boost genes, booster polypeptides, genome engineering components, and/or transgenes. Particularly preferred is the co-introduction via particle bombardment.

### Regeneration of a Plant Cell Into a Whole Plant

According to another aspect of the present invention, the genetically modified plant cells can be regenerated into a whole (fertile) plant. Thus, in a preferred aspect of the invention, the genetic modification of a plant cell is followed by a step of regenerating a plant. Accordingly, the present invention provides a method for producing a genetically modified plant comprising the steps:
a) genetically modifying a plant cell according to any of the above methods for genetic modification in a plant cell, and
b) regenerating a plant from the modified plant cell of step a),

Single or multiple cells proliferate and develop into tissues, organs, and eventually entire plants. In some embodiments, the produced plant does not contain any of the genome engineering components, boost genes, and booster polypeptides introduced, or co-introduced in step a). Step b) of regenerating a plant can for example comprise culturing the genetically modified plant cell from step a) on a regeneration medium.

The efficiency of plant regeneration or of increasing the regeneration ability of a plant cell can be improved by introducing into the plant cell any of the booster polypeptides, boost genes, nucleic acids, recombinant genes and DNA constructs described herein.

### Production of a Genetically Modified Plant

The present invention also provides a genetically modified plant obtained or obtainable by the above methods for producing a genetically modified plant or a progeny plant thereof. The genetically modified plant may comprise any of the genetically modified plant cells described herein.

In various embodiments, the produced plant does not contain any of the genome engineering components, boost genes, and booster polypeptides introduced or co-introduced into a plant cell used to generate the produced plant.

The present invention also provides a plant or a seed derived from the above-described genetically modified cells without a conventional selection. As used herein, “conventional selection” refers to any processes to select and purify the transformed cells from wild-type cells by using an integrated selection marker, e.g. antibiotic (e.g. kanamycin, hygromycin), or herbicide (e.g. phosphinothricin, glyphosate) resistance gene. Without a conventional selection, such a plant or seed may not have any of the genome engineering components integrated, and thus leads to transgene-free genetic modified plants.

The genetic modification can be a permanent and heritable change in the genome of the plant cell. Plant tissue culture and genome engineering can be carried out using currently available methods, comprising of microparticle bombardment, Agrobacterium transformation, electroporation, etc. Transformation and transgene expression may be monitored by use of a visible report gene, for example, the red fluorescent tDTomato gene (DT) that encodes an exceptionally bright red fluorescent protein with excitation maximum at 554 nm and emission maximum at 581 nm. The genome editing efficiency can be analyzed for instance by next generation sequencing (NGS), qPCR, marker capillary electrophoresis analysis, and Droplet Digital PCR. Site-specific modification was further conformed by Sanger sequencing.

### Cultivation Step

The plant cell into which boost genes, booster polypeptides, genome engineering components, and/or transgenes have been introduced, or co-introduced, can be cultivated under conditions allowing the genetic modification of the genome of said plant cell by activity of the genome engineering component in the presence of one or more of a boost gene, a booster polypeptide, and one or more transgenes.

As used herein, “genetic modification of the genome” includes any type of manipulation such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetic modification is an alteration in the regulatory sequence, such as a promoter, to result in increased or decreased expression of an operably linked endogenous coding region.

Conditions that are “suitable” for a genetic modification of the plant genome to occur, such as cleavage of a polynucleotide, or “suitable” conditions are conditions that do not prevent such events from occurring. Thus, these conditions permit, enhance, facilitate, and/or are conducive to the event. Depending on the respective genome engineering component (i), these conditions may differ.

In the method of the present invention, the plant cell is preferably transiently transformed with the genome engineering component (i) and the at least one compound (ii). As used herein, “transient transformation” refers to the transfer of a foreign material [i.e. a nucleic acid fragment, protein, ribonucleoprotein (RNP), etc.] into host cells resulting in gene expression and/or activity without integration and stable inheritance of the foreign material. Thus, the genome engineering component (i) is transiently active and/or transiently present in the plant cell. The genome engineering component is not permanently incorporated into the cellular genome, but provides a temporal action resulting in a modification of the genome. For example, transient activity and/or transient presence of the genome engineering component in the plant cell can result in introducing one or more double-stranded breaks in the genome of the plant cell, one or more single-stranded breaks in the genome of the plant cell, one or more base-editing events in the genome of the plant cell, or one or more of DNA methylation, histone acetylation, histone methylation, histone ubiquitination, histone phosphorylation, histone sumoylation, histone ribosylation or histone citrullination in the genome of the plant cell.

The introduction of one or more double-stranded breaks or one or more single-stranded breaks is preferably followed by non-homologous end joining (NHEJ) and/or by homology directed repair (HDR) of the break(s) through a homologous recombination mechanism.

The resulting modification in the genome of the plant cell can, for example, be selected from an insertion of a transgene, preferably an expression cassette comprising a transgene of interest, a replacement of at least one nucleotide, a deletion of at least one nucleotide, an insertion of at least one nucleotide, a change of DNA methylation, a change in histone acetylation, histone methylation, histone ubiquitination, histone phosphorylation, histone sumoylation, histone ribosylation, or histone citrullination or any combination thereof. According to a particularly preferred aspect of the invention, no exogenous genetic material related to the applied gene editing machinery/systems is stably integrated into the genome of the plant cell.

The genetic modification can be a permanent and heritable change in the genome of the plant cell.

### Optional Pre-Treatment

In various embodiments, pre-treatment of plant materials with one or more chemicals described in U.S. Provisional Application No. 62/685,626, incorporated herein by reference, can be included. Thus, the methods for genetic modification in a plant cell may further comprise a step of pretreatment of the plant cell, said pretreatment comprising culturing the plant cell or plant material comprising same in a medium containing (1) an epigenetically regulating chemical or an active derivative thereof, in particular the histone deacetylase inhibitor (HDACi) or the DNA methyltransferase inhibitor, or (2) a phytohormone or an active derivative thereof, or any combination thereof.

After the pretreatment step, the treated plant cells may be taken from the medium containing at least one of compounds (1) and (2) and used for co-introduction.

Exemplary, as for the histone deacetylase inhibitor TSA, the duration of the HDACis pre-treatment is from 10 minutes to 2 days, preferred 2.0 to 24 hours. TSA concentration for a pre-treatment is 1.0 nM to 1000 nM, preferred 10 nM to 100 nM. Hereafter the treated plant materials are transferred to HDACi-free medium and used for TSA co-introduction immediately (a prolonged TSA pre-treatment may cause non-selectively enhancement of cell regeneration, which may increase difficult in retrieving the bombarded and modified cells).

Similar conditions of pre-treatment can be applied for all types of compounds (1) and (2). Plant tissue culture and genome engineering can be carried out using currently available methods. Transient transformation and transgene expression may be monitored by use of the red fluorescent report gene tdTomato, which encodes an exceptionally bright red fluorescent protein with excitation maximum at 554 nm and emission maximum at 581 nm, or the green fluorescent report gene mNeonGreen, which encodes the brightest monomeric green or yellow fluorescent protein with excitation maximum at 506 nm and emission maximum at 517 nm. The genome editing efficiency can be analyzed for instance by next generation sequencing (NGS).

### Microparticles

In another aspect is provided a microparticle coated with at least one of the above booster polypeptides, nucleic acids, recombinant genes or DNA constructs. In some embodiments, the microparticle is further coated with a genome engineering component.

In another aspect is provided a kit for the genetic modification of a plant genome by microprojectile bombardment, comprising
(I) one or more microparticles, and
(II) means for coating the microparticles.

In some embodiments, the kit further comprises a means for coating the microparticles with a genome engineering component.

In various embodiments, the microparticle is coated with at least
(i) a booster polypeptide, or a nucleic acid encoding the booster polypeptide;
(ii) a transgene; and/or a genome engineering component.

In a particularly preferred embodiment of microparticle bombardment, the boost polypeptide and/or one or more boost genes can be co-delivered with the genome engineering components via microcarriers comprising gold particles having a size in a range of 0.4-1.6 micron (μm), preferably 0.4-1.0 μm. In an exemplary process, 10 ng-10 μg of DNA, preferably 50-1000 ng of DNA, coated onto 10-1000 μg of gold particles, preferably 50-300 μg, are used per one bombardment. Up to 10 bombardments (shots), preferred 1-4 shots, per one sample plate can be used for the delivery of foreign molecules into plant cells.

Boost factors (e.g., boost polypeptides or polynucleotides encoding such boost polypeptides) and genome engineering components can be delivered into target cells for example using a Bio-Rad PDS-1000/He particle gun or handheld Helios gene gun system. When a PDS-1000/He particle gun system used, the bombardment rupture pressures are from 450 psi to 2200 psi, preferably from 450 psi to 1100 psi, while the rupture pressures are from 100 psi to 600 psi for a Helios gene gun system. More than one chemical or construct can be co-delivered with genome engineering components into target cells simultaneously.

The microparticle coating can further comprise one or more coating layers. For example, a microparticle may contain a first coating layer comprising a boost factor and a second coating layer comprising the genome engineering component and the transgene. Alternatively, a microparticle may contain a coating layer comprising a boost factor and either the transgene or the genome engineering component.

Further, the invention provides a kit for the genetic modification of a plant genome by microprojectile bombardment, comprising
(I) above one or more microparticles, and
(II) means for coating the microparticles with at least a genome engineering component and (1) an epigenetically regulating chemical, e.g. a DNA methyltransferase inhibitor or a protein deacetylase inhibitor or an active derivative thereof, in particular a histone deacetylase inhibitor (HDACi), and/or (2) a phytohormone or an active derivative thereof.

Another aspect of the present invention is the use of a microparticle as described above for the biolistic transformation of a plant cell.

Subject matter of the present invention are also the plant cells that are obtained or obtainable by the methods described above. Accordingly, one embodiment of the invention is a genetically modified plant cell obtained or obtainable by the above method for genetic modification in a plant cell. The genetic modification in these plant cells compared to the original plant cells may, for example, include an insertion of a transgene, preferably an expression cassette comprising a transgene of interest, a replacement of at least one nucleotide, a deletion of at least one nucleotide, an insertion of at least one nucleotide, a change of DNA methylation, a change in histone acetylation, histone methylation, histone ubiquitination, histone phosphorylation, histone sumoylation, histone ribosylation, or histone citrullination or any combination thereof. Preferably, the genetically modified plant cell does not comprise any exogenous genetic materials stably integrated into the genome of the plant cell.

Genetically modified plant cells can be part of a whole plant or part thereof. Thus, the present invention also relates to a plant or plant part comprising the above genetically modified plant cell.

According to another aspect of the present invention, the genetically modified plant cells can be regenerated into a whole (fertile) plant. Thus, in a preferred aspect of the invention, the genetic modification of a plant cell is followed by a step of regenerating a plant. Accordingly, the present invention provides a method for producing a genetically modified plant comprising the steps:
a) genetically modifying a plant cell according to the above method for genetic modification in a plant cell, and
b) regenerating a plant from the modified plant cell of step a).

Step b) of regenerating a plant can for example comprise culturing the genetically modified plant cell from step a) on a regeneration medium.

Regeneration techniques rely on manipulation of certain phytohormones in a tissue culture growth medium, occasionally relying on a biocide and/or herbicide marker that can been introduced. Regeneration can be obtained from plant somatic cells, callus cells or embryonic cells and protoplasts derived from different explants, e.g. callus, immature or mature embryos, leaves, shoot, roots, flowers, microspores, embryonic tissue, meristematic tissues, organs, or any parts thereof. Such regeneration techniques are described generally in Klee (1987) Ann. Rev. of Plant Phys. 38:467486. Plant regeneration from cultured protoplasts is described in Evans et al., Protoplasts Isolation and Culture, Handbook of Plant Cell Culture, pp. 124-176, Macmillan Publishing Company, New York, 1983; and Binding, Regeneration of Plants, Plant Protoplasts, pp. 21-73, CRC Press, Boca Raton, 1985. To obtain whole plants from transformed or gene edited cells, the cells can be grown under controlled environmental conditions in a series of media containing nutrients and hormones, a process known as tissue culture. Once whole plants are generated and produce seed, evaluation of the progeny begins.

The present invention also provides a genetically modified plant obtained or obtainable by the above method for producing a genetically modified plant or a progeny plant thereof.

Further subject matter of the present invention is a plant cell or a seed derived from the above genetically modified plant.

Further subject matter of the present invention is a plant, plant cell or a seed derived from the above genetically modified cell without a marker gene-based selection. As used herein, “marker gene-based selection” refers to any processes to select, identify and/or purify the modified cells, in particular the transformed, gene edited or base edited cells, from wild-type cells by using an integrated selection marker (gene), e.g. antibiotic resistance gene (e.g. kanamycin resistance gene, hygromycin resistance gene), or herbicide resistance gene (e.g. phosphinothricin resistance gene, glyphosate resistance gene). Without such selection, such a plant, plant cell or seed may not have any of the genome engineering components integrated, which may yield (i) transgene-free genetic modified plants or (ii) modified plants which have integrated solely the transgene of interest.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Cray, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Sequences

Sequences

| SEQ ID NO: | Description |
|---|---|
| 1 | cDNA of KWS-RBP1 |
| 2 | protein of KWS-RBP1 |
| 3 | cDNA of ZmPLT5 |
| 4 | protein of ZmPLT5 |
| 5 | cDNA of AtPLT5 |
| 6 | protein of AtPLT5 |
| 7 | cDNA of ZmPLT7 (genotype A188) |
| 8 | protein of ZmPLT7 (genotype A188) |
| 9 | cDNA of AtPLT7 |
| 10 | protein of AtPLT7 |
| 11 | cDNA of TaRKD4 |
| 12 | protein of TaRKD4 |
| 13 | cDNA of AtRKD4 |
| 14 | protein of AtRKD4 |
| 15 | cDNA of ZmRKD4 |
| 16 | protein of ZmRKD4 |
| 17 | cDNA of TaRKD2 |
| 18 | protein of TaRKD2 |
| 19 | cDNA of AtRKD2 |
| 20 | protein of AtRKD2 |
| 21 | cDNA of ZmRKD2 |
| 22 | protein of ZmRKD2 |
| 23 | promoter of BdEF1 |
| 24 | pABM-BdEF1 |
| 25 | pABM-BdEF1_ZmPLT5 |
| 26 | pABM-BdEF1_ZmPLT7 |
| 27 | pABM-BdEF1_KWS-RBP1 |
| 28 | pABM-BdEF1_TaRKD4 |
| 29 | PGEP359 |
| 30 | pGEP324 |
| 31 | pAMK-BdEF_ZmWUS2 |
| 32 | BdEF1::ZmPLT5_expression_cassette |
| 33 | BdEF1::ZmPLT7_expression_cassette |
| 34 | BdEF1::KWS-RBP1_expression_cassette |
| 35 | BdEF1::TaRKD4_expression_cassette |
| 36 | BdEF1::ZmWUS2_expression_cassette |
| 37 | pUbi::LpCpf1_expression_cassette |
| 38 | pUbi::crRNA5_expression_cassette |
| 39 | cDNA of LbCpf1 |
| 40 | protein of LbCpf1 |
| 41 | crRNA5_target_HMG13 |
| 42 | crRNA5_target_sequence |
| 43 | pAMK-ZmWUS2-tDT-nosT |
| 44 | cDNA of ZmPLT7 (genotype B73) |
| 45 | protein of ZmPLT7 (genotype B73) |
| 46 | pZmWUS2::tDT-nosT expression cassette |
| 47 | cDNA of KWS-RBP2 |
| 48 | protein of KWS-RBP2 |
| 49 | pABM-BdEF1_KWS-RBP2 |
| 50 | BdEF1::KWS-RBP2_expression_cassette |

All patents, patent applications, and publications or public disclosures (including publications on internet) referred to or cited herein are incorporated by reference in their entirety.

### EXAMPLES

The present invention is further illustrated by the following examples. However, it is to be understood that the invention is not limited to such examples. The use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Transient Co-Expression of Boost Genes and Genes of Interest (GOI) by Co-Bombardment

### Gene Cloning and Construct Preparation

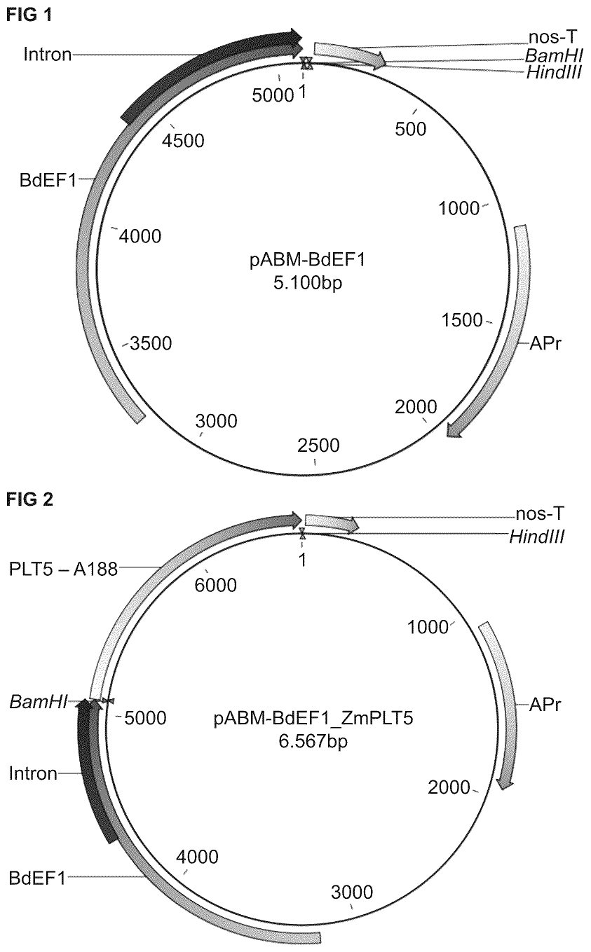

Maize PLT5 (ZmPLT5) and PLT7 (ZmPLT7) genes were cloned by RT-PCR using total RNA isolated from maize A188 immature embryos. Wheat RKD4 and KWS-RBP1 genes were maize-codon optimized from its protein sequence, and synthesized by Integrated DNA Technologies (IDT, San Diego, CA, USA). The boost gene fragments are cloned into expression vector pABM-BdEF1 (FIG. 1) at the cloning site of BamHI and HindIII, and expressed under the control of a BdEF1 promoter (pBdFE1) and a nos terminator (nos-T). pBdFE1 is a strong constitutive promoter from Brachypodium. The sequencing-confirmed construct maps are shown in FIGS. 2-5.

### Preparing Maize Immature Embryo for Bombardment

At 9-12 days post pollination, maize ears (i.e. A188 or Hi II) with immature embryos having a size of 0.8 to 1.8 mm, preferably 1.0-1.5 mm, were harvested. The ears were sterilized with 70% ethanol for 10-15 minutes. After brief air drying in a laminar hood, the top ˜⅓ of the kernels were removed from the ears with a shark scalpel, and the immature embryos were pulled out of the kernels carefully with a spatula. The fresh isolated embryos were placed onto the bombardment target area in an osmotic medium plate (see below) with scutellum-side up. The plates were wrapped with parafilm and incubated at 25° C. in the dark for 4 hours before bombardment.

### Particle Co-Bombardment

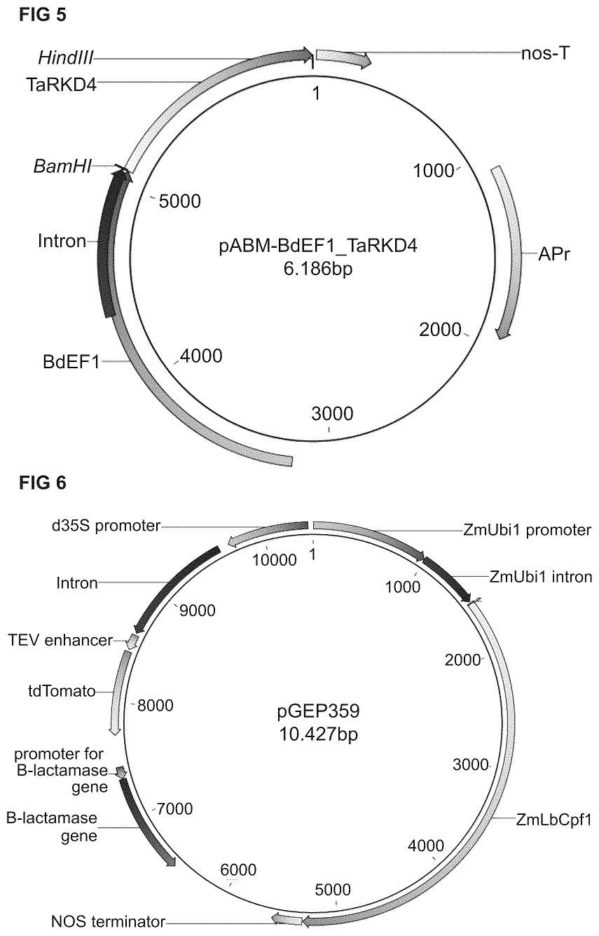

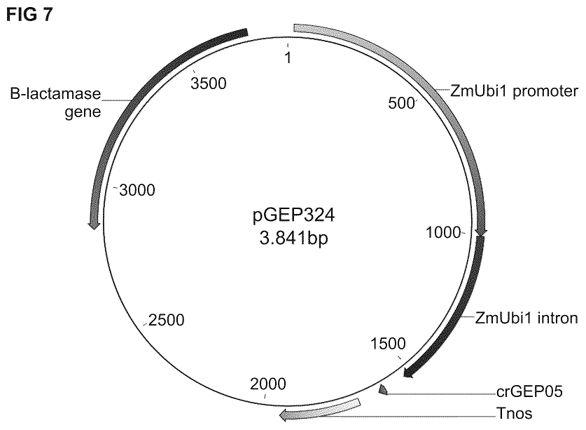

A particle bombardment gun and gold particles having a size of 0.4 or 0.6 microns (μm) were used to deliver DNA into the scutellum cells of maize immature embryos. The boost gene plasmids were premixed with genes of interest (GOI), e.g., genome editing constructs pGEP359 that harbor CRISPR nuclease Cpf1 and a tDT report gene (FIG. 6), and pGEP324 that contains the CRISPR guide RNA crRNA5 target to maize HMG13 (FIG. 7). For 10 shots, 1 mg of gold particle in 50% (v/V) glycerol (100 μg of gold particles per shot) in a total volume of 100 microliter (μl) was pipetted into a clear low-retention microcentrifuge tube. The mixture was sonicated for 15 seconds to suspend the gold particles. While vortexing at a low speed, the following were added, in order, to each 100 μl of gold particles: (a) up to 10 μl of DNA (1.0-10.0 μg total DNA of pre-mixed, 100-1000 ng per each shot), (b) 100 μl of 2.5 M CaCl₂ (pre-cold on ice), and (c) 40 μl of 0.1 M cold spermidine.

The lid was closed and the tube vortexed for 2-30 minutes at 0-10° C., and the DNA-coated gold particles were spun down. After washing in 500 μl of 100% ethanol two times, the pellet was resuspended in 120 μl of 100% ethanol. While vortexing at a low speed, 10 μl of co-coated gold particles were pipetted with a wide open 20 μl tip from the tube onto the center of the macrocarrier evenly. Since the particles tend to form clumps at this point, the gold particles were placed onto the macrocarriers as soon as possible, followed by air drying. Bombardment was conducted using a Bio-Rad PDS-1000/He particle gun. The bombardment conditions were: 28 mm/Hg vacuum, 450 or 650 psi rupture disc, 6 mm gap distance, the specimen platform is in the second position from the bottom in the chamber at a distance of 60 mm, three shots per sample (maize immature embryos) plate.

### Post Bombardment Observation and Embryo Culture

After bombardment, the embryos remained on the osmotic medium for another 16 hours. Transient transformation was examined using a fluorescence microscope for the tDT expression at excitation maximum 554 nm and emission maximum 581 nm 16-20 hours after bombardment. The embryos with dense fluorescent signals under a fluorescence microscope (FIG. 8) were selected and transferred from N6OSM onto a N6-5Ag plate (˜15 embryos per plate) with scutellum-face-up for callus induction (see below).

Osmotic medium: N6 salt, N6 vitamin, 1.0 mg/L of 2, 4-D, 100 mg/L of Casein, 0.7 g/L of L-proline, 0.2 M Mannitol (36.4 g/L), 0.2 M sorbitol (36.4 g/L), 20 g/L sucrose, 15 g/L of Bacto-agar, pH 5.8.

N6-5Ag: N6 salt, N6 vitamin, 1.0 mg/L of 2,4-D, 100 mg/L of Casein, 2.9 g/L of L-proline, 20 g/L sucrose, 5 g/L of glucose, 5 mg/L of AgNO3, 8 g/L of Bacto-agar, pH 5.8.

### Example 2. Transient Co-Expression of ZmPLT5 or ZmPLT7 Gene and KWS-RBP1 Promotes Early Embryogenesis and Regeneration in Maize Hi II Immature Embryo

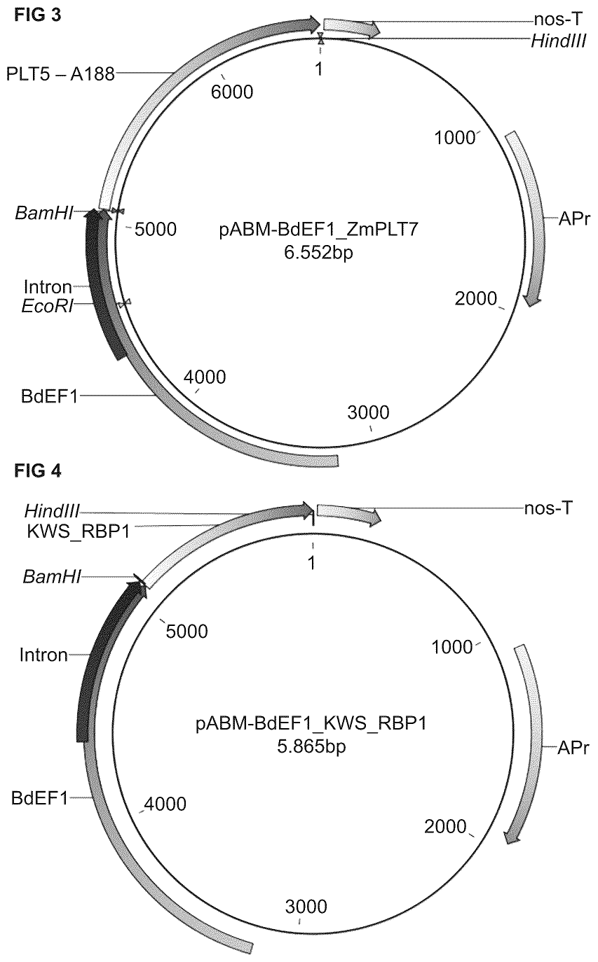

Transient co-delivery, embryo preparation and culturing are described above in Example 1. For each bombardment, four premixed DNA plasmids were coated onto 100 μg of gold particles having a size of 0.4 μm, and co-introduced into the scutellum cell of Hi II immature embryos at 650 psi rupture pressure. Four plasmids were premixed as follows for one bombardment:
100 ng of boost ZmPLT5 or ZmPLT7 (FIG. 2 and FIG. 3)
200 ng of KWS-RBP1 (FIG. 4)
100 ng of pGEP359 (FIG. 6)
150 ng of pGEP324 (FIG. 7)

The embryos with dense fluorescent signals under a fluorescence microscope (FIG. 8) were selected and transferred from N6OSM onto N6-5Ag for embryonic callus induction. The selected embryos were cultured in a N6-5Ag plate with the scutellum-face-up (roughly 15 embryos per plate) at 27° C. in dark for 14 in dark. Embryogenic callus induction was monitored by observation under a dissection microscope. Specifically, the boost effect on cell division and regeneration was measured by its capability to induce embryo formation 5-7 days after bombardment by visual observation under a fluorescence microscope.

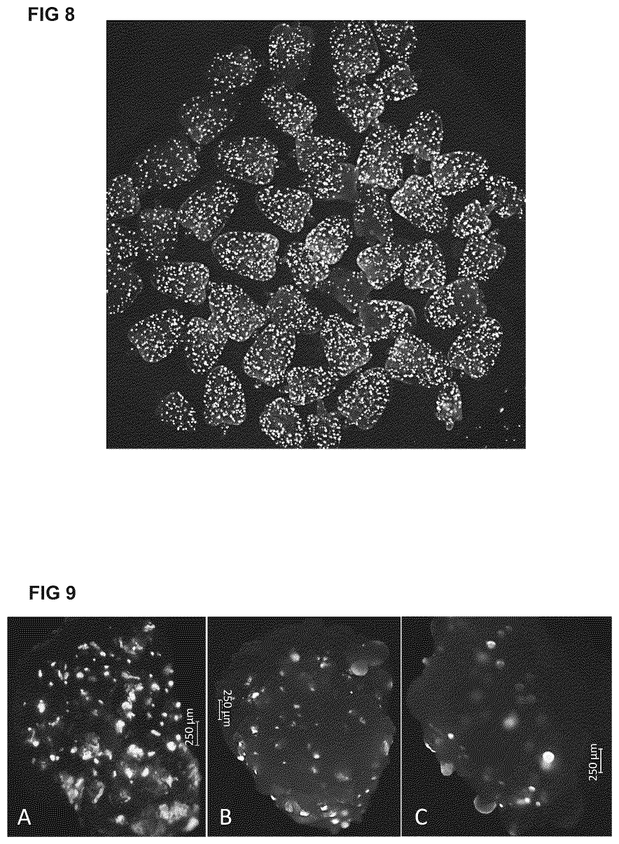

FIG. 9 shows that co-expression of ZmPLT5 (FIG. 9B) or ZmPLT7 (FIG. 9C) and KWS-RBP1 by microprojectile bombardment significantly promotes embryogenic callus induction in maize Hi II immature embryos. Compared to the image in FIG. 9A from the bombardment without a booster, the images in FIG. 9B and FIG. 9C show multiple embryonic structure formed and emerging 5 days after the particle bombardment.

### Example 3. Transient Co-Expression of ZmPLT5 or ZmPLT7 and KWS-RBP1 Improves Stable Transformation of a Co-Delivered Report Gene in Maize Hi Immature Embryo

Maize embryo preparation, transient bombardment, and embryonic callus induction are described in Examples 1 and 2. The embryos were cultured in N6-5Ag medium at 27° C. in the dark for 14 days. tDT fluorescence was used to monitor embryogenic callus induction and stable transformation by observation under a fluorescent microscope. Specifically, the boost effect was measured by its capability to increase transformation frequency (TF) of the tDT report gene 12 days after bombardment without a selection.

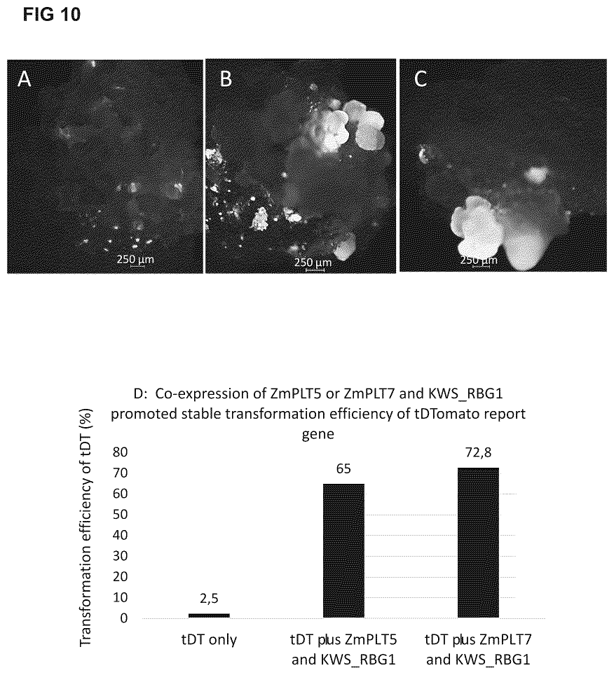

The strong and uniformed tDT fluorescent signals from the emerging embryonic structures in FIG. 10 indicated integration and stable transformation of tDT gene. Stable transformation frequency is defined as the number of embryos with at least one stable tDT fluorescent structures induced from 100 embryos initially used. Stable transformation frequency was measured 12 days after bombardment.

Transient co-expression of ZmPLT5 and KWS-RBP1 genes led to a 65% transformation frequency of the tDT gene (26-fold increase compared to the control without a booster), while the co-delivery of tDT with ZmPLT7 and KWS-RBP1 gave a 72.8% transformation frequency of the tDT gene (over 29-fold increase compared to the control) (FIG. 10D). The results from FIG. 10 suggest that transient co-expression of (i) ZmPLT5 or ZmPLT7 and (ii) KWS-RBP1 promote stable transformation frequency in maize Hi II immature embryos.

Stable transformation occurs at the single cell level, in which initially transferred DNA integrated into the genome of a host cell. To recover a homogenous transgenic plant, a few rounds of selection were needed to identify and purify the cells with the stable DNA integration. Without a booster, a stable transformation took a few weeks to develop (depending on the speed of cell proliferation), e.g. 4-8 weeks in maize. Compared to traditional transformation without a booster, the stable transformation shown in FIG. 10 was achieved only 12 days after bombardment with boost genes. Therefore, transient co-expression of ZmPLT5 or ZmPLT7 and KWS-RBP1 genes reduced the time needed for generating a stable transformation, and result in fast and highly efficient transformation in maize.

### Example 4.1 Transient Co-Expression of ZmPLT5 or ZmPLT7 Gene and KWS-RBP1 Promotes Early Embryogenesis and Regeneration in Maize A188 Immature Embryo

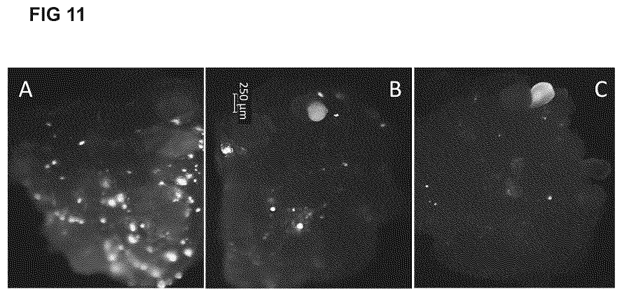

The experimental procedure was carried out as described in Example 2. The results were recorded seven days after bombardment. The results are shown in FIG. 11, which demonstrates that transient co-expression of ZmPLT5 (FIG. 11B) or ZmPLT7 (FIG. 11C) and KWS-RBP1 by microprojectile bombardment significantly promotes embryogenic structure induction in maize A188 immature embryos. Compared to the image in FIG. 11A without a booster, the images in FIG. 11B and FIG. 11C show multiple embryonic structures were formed. The structures emerged seven days after the particle bombardment.

### Example 4.2 Transient Co-Expression of ZmPLT5 Gene and KWS-RBP2 Promotes Early Embryogenesis and Regeneration in Maize A188 Immature Embryo

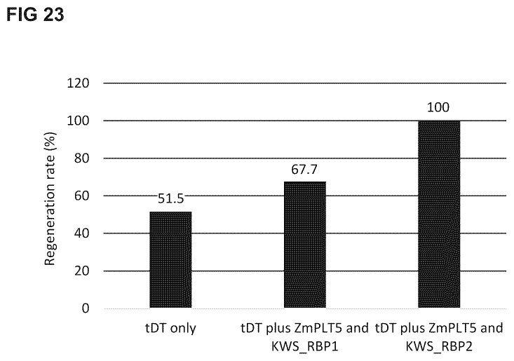

The experimental procedure was carried out as described in Example 2. The results were recorded ten days after bombardment. The results are shown in FIG. 23, which demonstrates that transient co-expression of ZmPLT5 and KWS-RBP2 by microprojectile bombardment significantly promotes embryogenic structure induction in maize A188 immature embryos. The regeneration rate (in %) after co-expression of ZmPLT5 gene and KWS-RBP2 is even higher than the rate observed after co-expression of ZmPLT5 gene and KWS-RBP1. Regeneration rate is defined as the number of embryos giving at least one plant regenerated from 100embryos initially used. Data was record 10 days after bombardment.

### Example 5.1 Transient Co-Expression of ZmPLT5 or ZmPLT7 Gene and KWS-RBP1 Promotes Early Stable Transformation of a Co-Delivered Report Gene in Maize A188 Immature Embryo

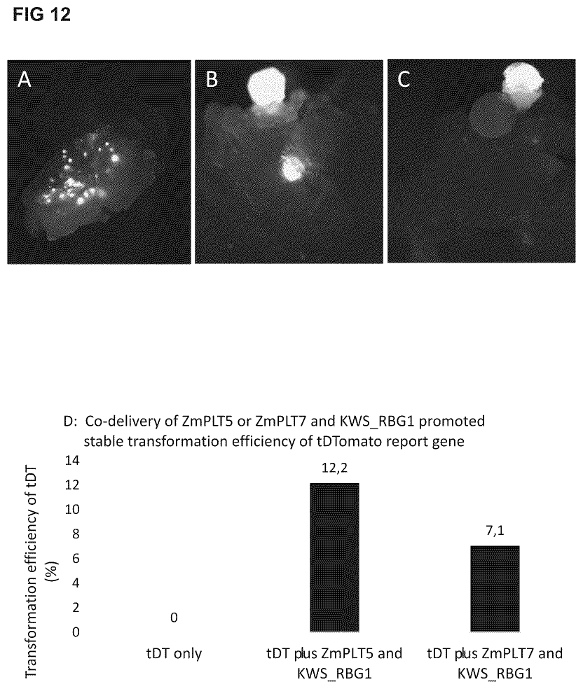

The experimental procedure was carried out as described in Example 3. The results were recorded 16 days after bombardment. The strong and uniformed tDT fluorescent signals from the emerging embryonic structures in FIGS. 12B and 12C indicate integration and stable transformation of tDT gene. Compared to the image in FIG. 12A, the red fluorescence images in FIGS. 12B and 12C illustrate that co-expression of ZmPLT5 (FIG. 12B) or ZmPLT7 (FIG. 12C) with KWS-RBP1 significantly improves stable transformation of the report gene in maize A188 immature embryos.

After 16 days from bombardment of A188 immature embryos without selection, no stable transformation was observed from the control without a booster. Compared to the control, co-bombardment of the tDT construct with ZmPLT5 and KWS-RBP1 led to 12.2% of the transformation frequency, while co-bombardment with ZmPLT7 and KWS-RBP1 gave 7.1% of transformation frequency of tDT report (FIG. 12D) 16 days after bombardment in maize A188.

### Example 5.2 Transient Co-Expression of ZmPLT5 and KWS-RBP2 Promotes Early Stable Transformation of a Co-Delivered Report Gene in Maize A188 Immature Embryo

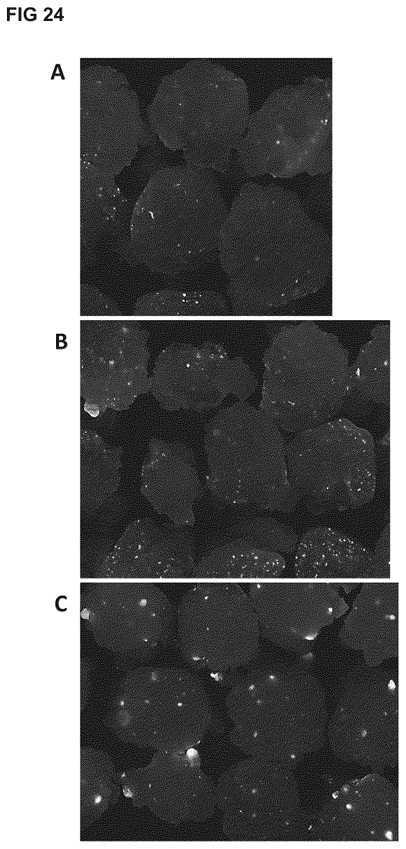

The experimental procedure was carried out as described in Example 3. The results were recorded 10 days after bombardment. The strong and uniformed tDT fluorescent signals from the emerging embryonic structures in FIGS. 24B and 24C indicate integration and stable transformation of tDT gene. Compared to the image in FIG. 24A, the red fluorescence images in FIGS. 24B and 24C illustrate that co-expression of ZmPLT5 with KWS-RBP1 and ZmPLT5 with KWS-RBP2 significantly improves stable transformation of the report gene in maize A188 immature embryos.

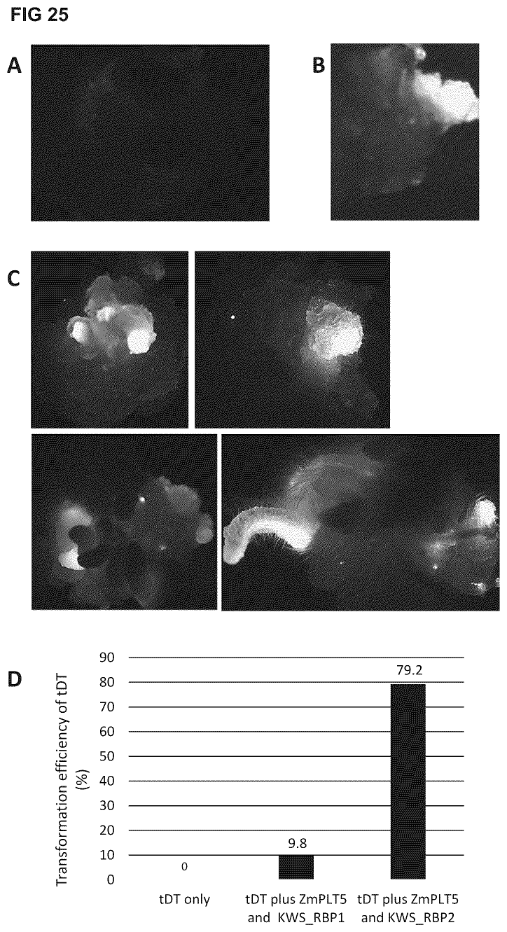

After 16 days from bombardment of A188 immature embryos without selection, no stable transformation was observed from the control without a booster (tDT only; FIG. 25A). Compared to the control, co-bombardment of the tDT construct with ZmPLT5 and KWS-RBP1 (FIG. 25B) led to 9.8% of the transformation frequency, while co-bombardment with ZmPLT5 and KWS-RBP2 (FIG. 25C) gave 79.2% of transformation frequency of tDT report (FIG. 25D) 16 days after bombardment in maize A188.

### Example 6. Wheat RKD4 Activates Maize WUSCHEL (WUS) Expression

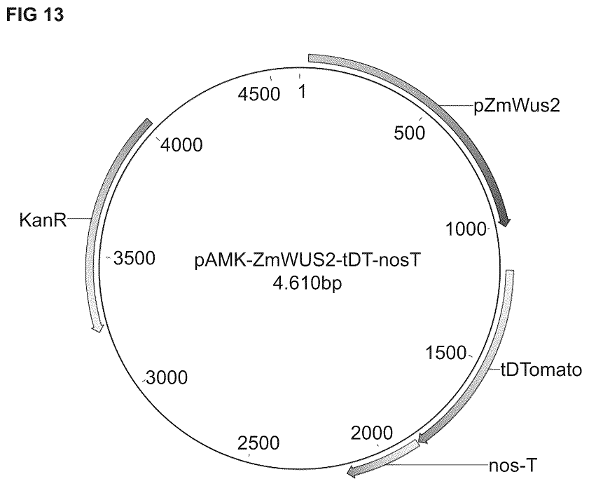

Homeobox domain transcriptional factor WUSCHEL (WUS) plays an important role in establishing and maintaining of shoot meristem. To identify boost factors that promote endogenous WUS2 expression, the maize WUSCHEL 2 promoter report construct (pAMK-ZmWUS2-tDT-noT) (SEQ ID NO: 43; FIG. 13) was used to illustrate maize WUS2 promoter activity. The maize WUS2 promoter (pZmWUS2) drove expression of the tDT report gene in this report construct (FIG. 13). The WUS2 promoter report construct was co-bombarded with boost factors individually in maize immature embryos and leaf segments.

Fresh leaf segments of 1-2 cm in length were prepared from the in vitro-cultured maize A188 seedling of 10-14 days old, and placed on the Osmotic medium with abaxial side up for 4 hours. For co-bombardment, two plasmids (100 ng of ZmWUS2 promoter report (FIG. 13) and 100 ng of boost construct, e.g. TaRKD4 (FIG. 5)) were premixed and coated onto 100 μg of gold particles size 0.4 μm. Immature embryo preparation, bombardment, and post-bombardment culturing were carried out as described in Example 1 and Example 2. Red fluorescence showing tDT expression was monitored using a fluorescent microscope started at 16 hours after bombardment.

WUS is transcribed specifically in the organization center (OC) of plant shoot apical meristem (SAM) and controls stem cell identity in the SAM.

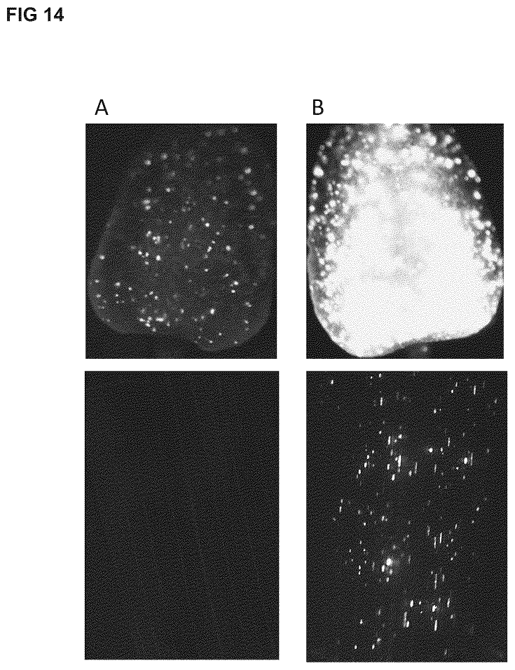

Bombardment with the ZmWUS2 promoter report only (pZmWUS2 report only) did not result in any tDT fluorescent signals from the bombarded leaf samples at any time during the after-bombardment culture (16 hours to 7 days). However, when co-bombarded with wheat RKD4 construct (FIG. 5), the tDT signal was detected in the leaf segments around 36 hours after bombardment, and peaked around 44 hours after bombardment (the bottom panel in FIG. 14B). Compared to the control bombardment with the WUS promoter reporter only, in which only weak tDT signals were noticed from the immature embryos (the top panel in FIG. 14A), extremely strong red fluorescent signals were observed from the embryos co-bombarded with the WUS promoter reporter and wheat RKD4 construct (the top panel in FIG. 14B). These results suggest wheat RKD4 strongly activate maize WUS2 genes. Images were taken 44 hours after bombardment.

### Example 7. Transient Co-Expression of TaRKD4 and KWS-RBP1 Promotes Early Embryogenesis From Maize Hi II Immature Embryo

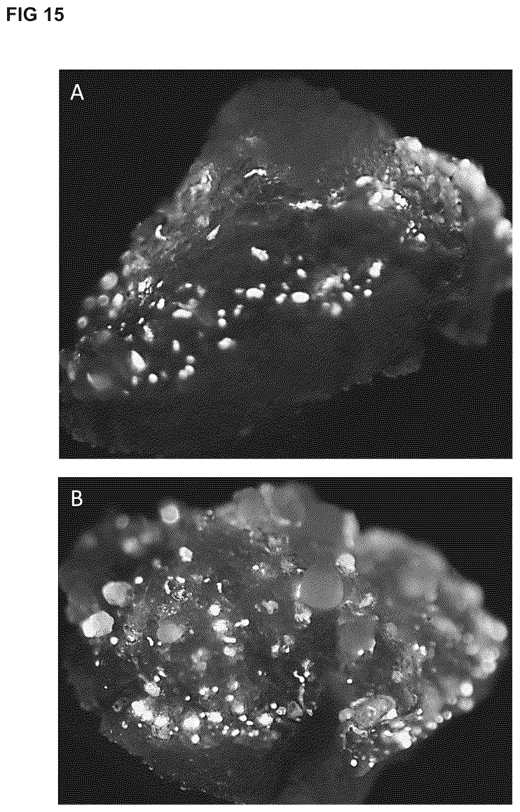

FIG. 15 shows that co-expression of wheat RKD4 (FIG. 5) and KWS-RBP1 (FIG. 4) by microprojectile bombardment significantly promotes embryogenic structure induction in maize Hi II immature embryos. The experiment was conducted as described in Example 2, with results recorded 5 days after bombardment. Compared to the image in FIG. 15A without a booster, the images in FIG. 15B show multiple embryonic structure were formed and emerging 5 days after the particle bombardment. Images were taken 5 days after the particle bombardment.

### Example 8. Transient Co-Expression of TaRKD4 and KWS-RBP1 Promotes Early Stable Transformation of a Co-Delivered Report Gene From Maize Hi II Immature Embryo

The experiment was conducted as described in Example 3, with results recorded 12 days after bombardment.

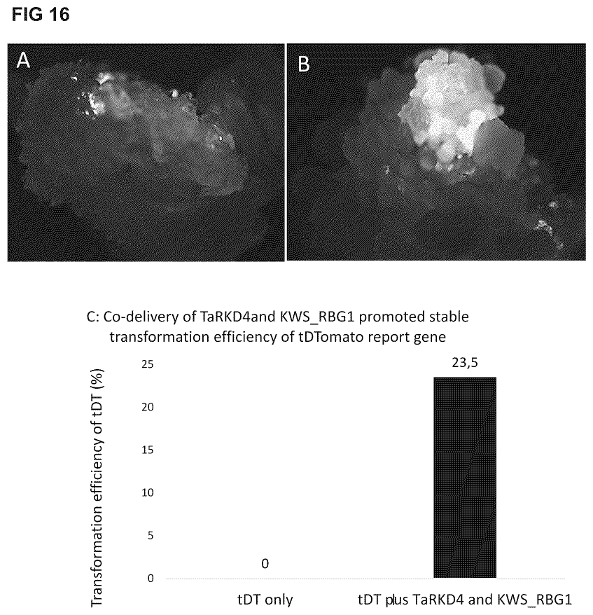

The strong and uniformed tDT fluorescent signals from the emerging embryonic structures in FIG. 16B indicate integration and stable transformation of tDT gene. Compared to the image in FIG. 16A, the red fluorescence images in FIG. 16B illustrate that co-delivery of TaRKD4 and KWS-RBP1 significantly improves stable transformation of the report gene in maize Hi II immature embryos.

12 days after bombardment of Hi II immature embryos without a selection, no stable transformation was observed from the control bombardment without a booster. Compared to the control, co-bombardment of the tDT construct with TaPLT4 and KWS-RBP1 led to 23.5% of the transformation frequency of the tDT report (FIG. 16C).

### Example 9. Transient Co-Expression of TaRKD4 and KWS-RBP1 Promotes Early Embryogenesis From Maize A188 Immature Embryo

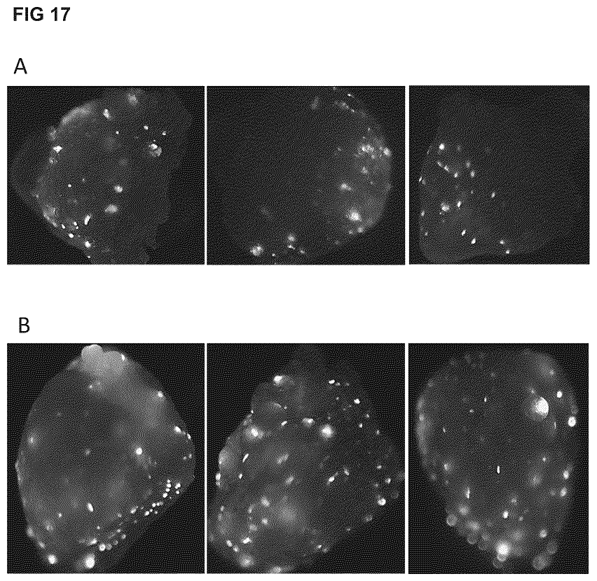

The experiment was conducted as described in Example 3, with results recorded 5 days after bombardment. FIG. 17 shows that co-delivery of TaRKD4 (FIG. 5) and KWS-RBP1 (FIG. 4) by microprojectile bombardment significantly promotes embryogenic structure induction in maize A188 immature embryos. Compared to the image in FIG. 17A without a booster, the images in FIG. 17B show multiple embryonic structure were formed and emerged 5 days after the particle bombardment. Images were taken 5 days after the particle bombardment.

### Example 10. Transient Co-Expression of TaRKD4 and KWS-RBP1 Promotes Early Stable Transformation of a Co-Delivered Report Gene From Maize A188 Immature Embryo

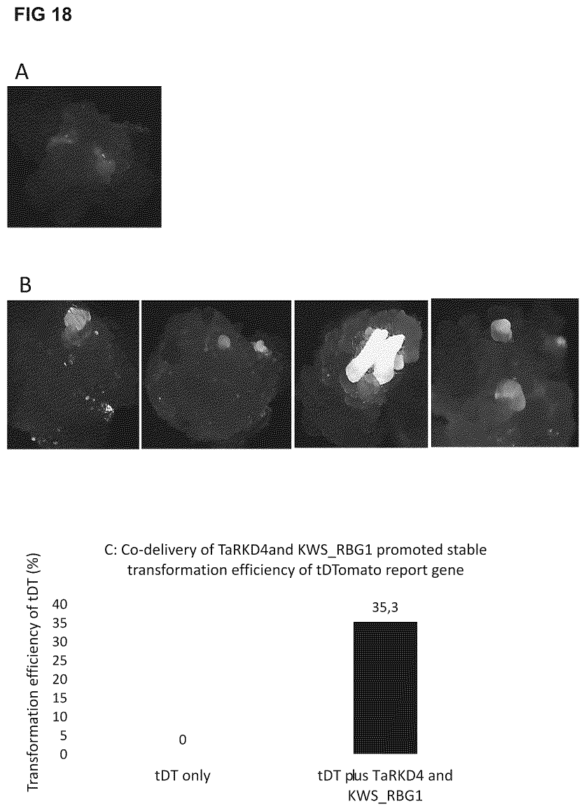

The experiment was conducted as described in Example 3, with results recorded 14 days after bombardment. Strong and uniform tDT fluorescent signals from the emerging embryonic structures in FIG. 18B indicate integration and stable transformation of the tDT gene. Compared to the image in FIG. 18A, the red fluorescence images in FIG. 18B illustrate that co-delivery of TaRKD4 and KWS-RBP1 significantly improves stable transformation of the report gene in maize A188 immature embryos.

No stable tDT fluorescent structure was observed from the control bombardment without a booster at 14 days after bombardment of A188 immature embryos without a selection. Compared to the control, co-bombardment of the tDT construct with TaRKD4 and KWS-RBP1 led to 35.5% of the transformation frequency of tDT report from A188 immature embryo (FIG. 18C).

### Example 11. Co-Expression of the Boost Genes With Genome Editing Components Promotes Transient Genome Editing in Maize

For embryo preparation, bombardment, and post-bombardment embryo culture, the procedures described in Example 1 and Example 2 were carried out. After callus induction in N6-5Ag medium for 14 days (Hi II) or 18 days (A188), the fast-growing embryogenic calluses from the bombarded scutellum surface of the embryos were picked and transferred onto MRM1 medium (see below) for embryo maturation. After about two weeks of culturing in MRM1 medium at 25° C. in the dark, mature embryos were moved onto MSO medium (see below) for embryo germination in phytotray in light at 25° C. After about 10 days of culturing in MSO medium, the regenerated plantlets were ready for molecular analysis and were transferred to soil. An approximately 5 mm leaf tip from all the leaves of a regenerated plantlet were collected for DNA extraction. The site-specific genome modification from the regenerated plants was screened by Taqman qPCR, marker capillary electrophoresis, and confirmed by Digital PCR, next generation sequencing (NGS), and Sanger sequencing. DNA integration was examined by qPCR.

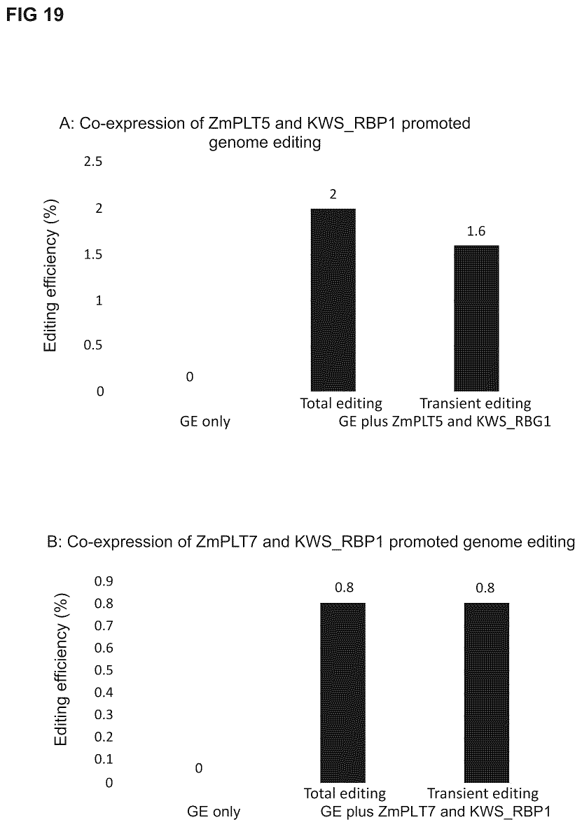

Without a booster, genome editing using the Cpf1 (pGEP359) and crRNA5 (pGEP324) did not result in any detectable editing event by transient expression with a selection (GE only) (FIG. 19). However, with co-expression with ZmPLT5 and KWS-RBP1 (GE plus ZmPLT5 and KWS-RBP1), 1% of transient genome editing efficiency was achieved (FIG. 19A), and 0.8% transient genome editing efficiency was also obtained when co-expressed with ZmPLT7 and KWS-RBP1 (GE plus ZmPLT5 and KWS-RBP1) (FIG. 19B). These results suggest the booster ZmPLT5, ZmPLT7, and KWS-RBP1 improve transient genome editing.

### Media

MRM1: MS Salts+MS vitamins+100 mg/L of myoinositol+6% sucrose+9 g/L of Bactoagar, PH 5.8
MS0: MS Salts+MS vitamins+2 g/L of myoinositol+2% sucrose+8 g/L of Bactoagar, pH 5.8

### Example 12. Homogenously Edited Plants can be Recovered by Transient Co-Expression of Genome Editing Components With the Boost Genes in Maize

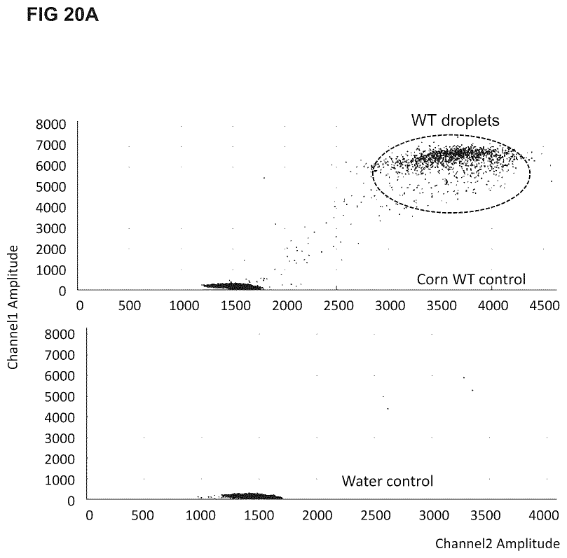

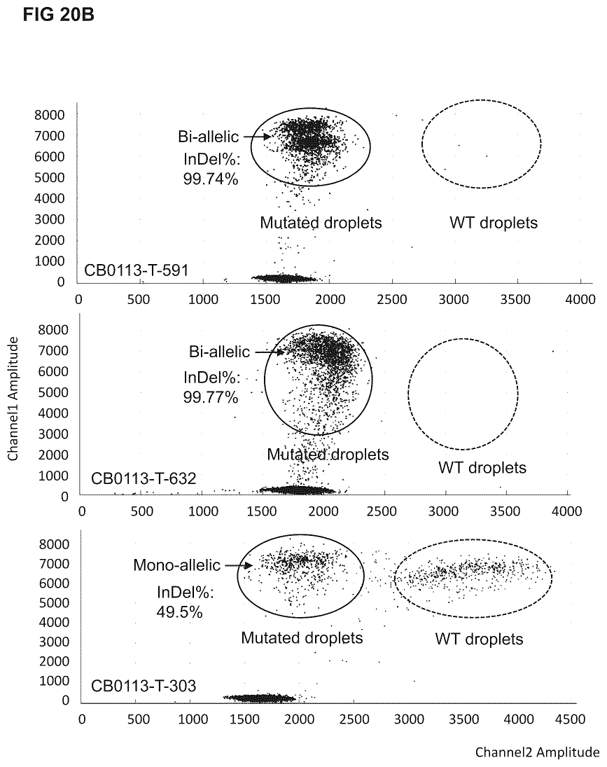

Droplet Digital PCR (ddPCR) was performed with transient co-expression of the boost genes and genome editing components without a selection. The site-specific InDel rates around 50% and 100% indicate a mono-allelic and bi-allelic modification, respectively. The data in FIG. 20A are results from a negative control with Droplet Digital PCR using water (bottom) or the wild type DNA (WT droplets). FIG. 20B shows the results from Droplet Digital PCR performed on edited T0 plants derived from transient co-expression of boosters and genome editing components. The top and middle graphs show a near 100% InDel rate from two edited T0 plants, indicating homogenous bi-allelic modification, while the bottom graph illustrates a homogenous mono-allelic edited event.

Without wishing to be bound by theory, genetic modification occurs at single cell level. To recover a homogenously modified plant, a selection is normally required to isolate the cells with a modification and remove wild-type cells. A conventional selection generally involves using an integrated selection marker, e.g. antibiotic (e.g. kanamycin, hygromycin), or herbicide (e.g. phosphinothricin, glyphosate) resistance gene. Without an integrated selection marker as the case in transient genome editing, regenerated plants will most likely be chimeric.

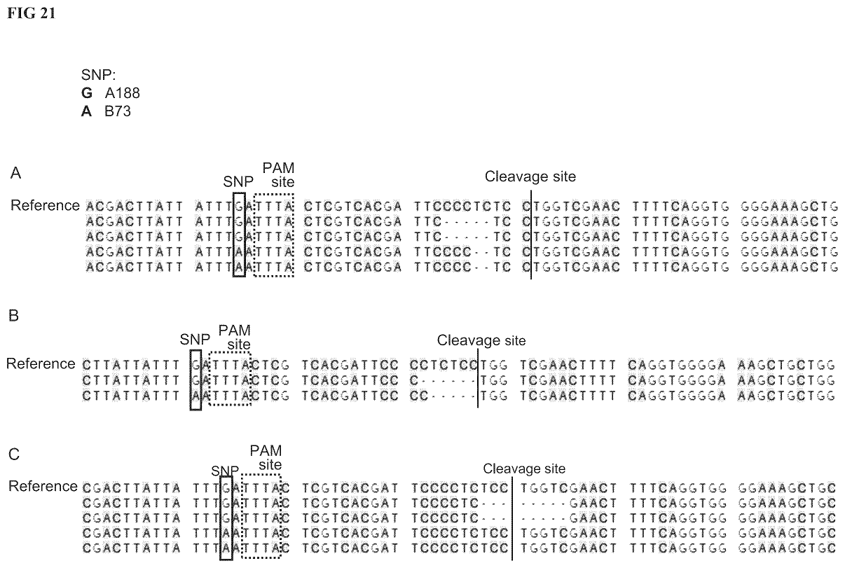

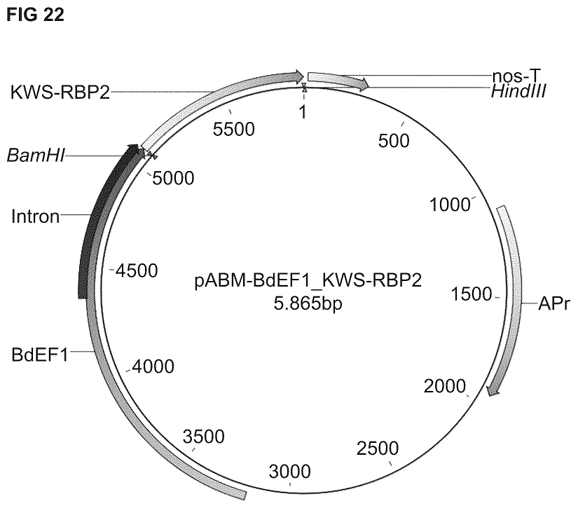

In contrast, the Droplet Digital PCR (ddPCR) results shown in FIG. 20 suggest that homogenous genome editing can be achieved by transient co-expression of genome editing components with the boost genes without a selection. An around 50% or 100% InDel rate from all the edited plants indicate a homogenous mono-allelic or bi-allelic modification. Sanger sequencing results further confirm the ddPCR results (FIG. 21). These results suggest that transient co-expression with the boost genes can lead to plant regeneration from single cell.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1-33. (canceled)

34. A microparticle coated with a coating comprising:
a) a booster polypeptide comprising the amino acid sequence of SEQ ID NO: 2 or 48, or an amino acid sequence at least 93% identical to SEQ ID NO: 2 or 48;
b) a nucleic acid encoding the booster polypeptide;
c) a nucleic acid encoding the booster polypeptide comprising the amino acid sequence of SEQ ID NO: 2, or an amino acid sequence at least 93% identical to SEQ ID NO: 2, the nucleic acid comprising a coding sequence selected from the group consisting of:
(i) a nucleic acid comprising the nucleotide sequence of SEQ ID NO: 1; and
(ii) a nucleic acid comprising a nucleotide sequence at least 93% identical to SEQ ID NO: 1;
d) a nucleic acid encoding the booster polypeptide comprising the amino acid sequence of SEQ ID NO: 48, or an amino acid sequence at least 93% identical to SEQ ID NO: 48, the nucleic acid comprising a coding sequence selected from the group consisting of:
(i) a nucleic acid comprising the nucleotide sequence of SEQ ID NO: 47; and
(ii) a nucleic acid comprising a nucleotide sequence at least 93% identical to SEQ ID NO: 47;
e) a recombinant gene comprising the nucleic acid, optionally linked to a heterologous promoter; or
f) a DNA construct comprising the nucleic acid.

35. The microparticle of claim 34, wherein the microparticle is further coated with a genome engineering component.

36. A kit for the genetic modification of a plant genome by microprojectile bombardment, the kit comprising
(I) one or more microparticles, and
(II) means for coating the microparticles of claim 34.

37. The kit of claim 36, further comprising a means for coating the microparticles with a genome engineering component.

38-41. (canceled)

42. The microparticle of claim 35, wherein the microparticle is further coated with an epigenetically regulating chemical.

43. The microparticle of claim 42, wherein the epigenetically regulating chemical is a histone deacetylase inhibitor (HDACi).

44. The microparticle of claim 42, wherein the HDACi is selected from the group consisting of trichostatin A (TSA), N-Hydroxy-7-(4-dimethylaminobenzoyl)-aminoheptanamide (M344), and suberoylanilide hydroxamic acid (SAHA).

45. The microparticle of claim 42, wherein the epigenetically regulating chemical is a DNA methyltransferase inhibitor.

46. The microparticle of claim 35, wherein the microparticle is further coated with a phytohormone.

47. The microparticle of claim 46, wherein the phytohormone is selected from the group consisting of 2,4-Dichlorophenoxyacetic acid (2,4-D), 6-Benzylaminopurine (6-BA), and Zeatin.


SEQUENCE LISTING (59 sequences)

SEQ ID NO: 1 - DNA, 783 bp, synthetic construct
  misc_feature 1..783: note = Description of Artificial Sequence: Synthetic polynucleotide
  misc_feature 1..783: note = coding sequence of KWS_RBP1
  source 1..783: mol_type = other DNA
atggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattga

SEQ ID NO: 2 - protein, 260 aa, synthetic construct
  REGION 1..260: note = Description of Artificial Sequence: Synthetic polypeptide
  REGION 1..260: note = protein of KWS_RBP1
MESGSGTAAGSGYVYRQPGSTRWNPTAEQLSLLREIYYRNGLRTPTADEIRQISSKLSRYGKIEGKNVYNWFQNRRAREKRKQRLSTIGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKILQLFPLHNNEDQTLIKSDKEIYCLGSCEKKMDLSPLGHSGSQRASALDLCLSLGNESCGLHDN

SEQ ID NO: 3 - DNA, 1485 bp, synthetic construct
  misc_feature 1..1485: note = Description of Artificial Sequence: Synthetic polynucleotide
  misc_feature 1..1485: note = cDNA of PLT5
  source 1..1485: mol_type = other DNA
atggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatga

SEQ ID NO: 4 - protein, 492 aa, Zea mays
MDTSHHYHPWLNFSLAHHCDLEEEERGAAAELAAIAGAAPPPKLEDFLGGGVATGGPEAVAPAEMYDSDLKFIAAAGFLGGSAAAAATSPLSSLDQAGSKLALPAAAAAPAPEQRKAVDSFGQRTSIYRGVTRHRWTGRYEAHLWDNSCRREGQSRKGRQESEGGYDKEEKAARAYDLAALKYWGSSTTTNFPVAEYEKEVEEMKNMTRQEFVASLRRKSSGFSRGASIYRGVTRHHQHGRWQARIGRVAGNKDLYLGTFSTEEEAAEAYDIAAIKFRGLNAVTNFEISRYNVETIMSSNLPVASMSSSAAAAAGGRSSKALESPPSGSLDGGGGMPVVEASTAPPLFIPVKYDQQQQEYLSMLALQQHHQQQQAGNLLQGPLVGFGGLYSSGVNLDFANSHGTAAPSSMAHHCYANGTASASHEHQHQMQQGGENETQPQPQQSSSSCSSLPFATPVAFNGSYESSITAAGPFGYSYPNVAAFQTPIYGME

SEQ ID NO: 5 - DNA, 1677 bp, synthetic construct
  misc_feature 1..1677: note = Description of Artificial Sequence: Synthetic polynucleotide
  misc_feature 1..1677: note = cDNA of AtPLT5
  source 1..1677: mol_type = other DNA
atgaagaacaataacaacaaatcttcttcttcttctagctatgattcttctttgtctccttcttcttcatcctcctcccaccagaactggctctctttctctctctccaacaataacaacaacttcaattcttcctcaaaccctaatctcacttcctccacatcagatcatcatcatcctcacccttctcacctctctctctttcaagctttctccacttctccagtcgaacggcaagatgggtcaccgggagtttcacccagcgatgccacggcggttctttccgtataccccggcggtcctaaacttgagaacttcctcggcggaggagcctcaacgacgacaacaagaccaatgcaacaagtgcaatctcttggcggcgttgtcttctcttccgacctacagccaccgcttcatcctccgtccgccgccgagatctacgactctgagctcaagtcaatagccgctagcttcctaggaaactactccggtggacactcgtcggaggtctctagcgtacataaacaacaaccgaatcctctagctgtctcagaggcttcgcctactccgaagaagaacgtagagagttttggacaacgtacctcgatttatagaggagtcacaagacatagatggactggaagatacgaagctcatctatgggataatagttgccgaagagaaggccaaagcagaaaaggaagacaagtttatttaggtggttatgataaggaagataaagcagctagagcttacgaccttgcagctcttaagtattggggtcctacaactacgactaatttcccgatatcaaattacgaatctgaacttgaagaaatgaaacacatgactcgacaagagttcgttgcttctttaagacggaaaagcagtggattctctaggggtgcctccatgtacagaggcgtcactagacatcatcagcatggtcgatggcaggcacgaattggaagagttgcaggcaacaaagacctttatcttggcacatttagcactcaagaggaagctgcagaagcttatgatatagcagcgatcaaattccgcggtctaaatgcagtcaccaatttcgacatcagtcgatatgatgtcaaatcaattgctagctgtaatctccctgtgggtggactaatgcctaaaccttctccagcaaccgcagcggctgacaaaaccgttgatctttctccatccgactctccatctctaaccacaccgtccctcacgttcaatgtggcaacaccggtcaatgaccatggaggaactttttaccacactggtataccaatcaaaccagacccggctgatcattattggtccaacatctttggattccaggcaaacccgaaagcagaaatgcgaccattagcaaactttgggtcggatcttcataacccttctcctggttatgctataatgccggtaatgcaggaaggtgaaaacaactttggtggtagttttgttgggtctgatgggtataacaatcattccgctgcatcgaacccggtctcagcaattccgctgtcctcgacaactacaatgagtaacggtaacgaagggtatggtggaaacataaactggattaataacaacatttcaagttcttaccaaactgcaaaatcaaatctctctgttttgcacacaccggtttttgggttggaatga

SEQ ID NO: 6 - protein, 558 aa, Zea mays
MKNNNNKSSSSSSYDSSLSPSSSSSSHQNWLSFSLSNNNNNFNSSSNPNLTSSTSDHHHPHPSHLSLFQAFSTSPVERQDGSPGVSPSDATAVLSVYPGGPKLENFLGGGASTTTTRPMQQVQSLGGVVFSSDLQPPLHPPSAAEIYDSELKSIAASFLGNYSGGHSSEVSSVHKQQPNPLAVSEASPTPKKNVESFGQRTSIYRGVTRHRWTGRYEAHLWDNSCRREGQSRKGRQVYLGGYDKEDKAARAYDLAALKYWGPTTTTNFPISNYESELEEMKHMTRQEFVASLRRKSSGFSRGASMYRGVTRHHQHGRWQARIGRVAGNKDLYLGTFSTQEEAAEAYDIAAIKFRGLNAVTNFDISRYDVKSIASCNLPVGGLMPKPSPATAAADKTVDLSPSDSPSLTTPSLTFNVATPVNDHGGTFYHTGIPIKPDPADHYWSNIFGFQANPKAEMRPLANFGSDLHNPSPGYAIMPVMQEGENNFGGSFVGSDGYNNHSAASNPVSAIPLSSTTTMSNGNEGYGGNINWINNNISSSYQTAKSNLSVLHTPVFGLE

SEQ ID NO: 7 - DNA, 1470 bp, synthetic construct
  misc_feature 1..1470: note = Description of Artificial Sequence: Synthetic polynucleotide
  misc_feature 1..1470: note = cDNA of ZmPLT7
  source 1..1470: mol_type = other DNA
atggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtag

SEQ ID NO: 8 - protein, 489 aa, Zea mays
MDMDMSSAYPHHWLSFSLSNNYHHGLLEAFSNSSGTPLGDEPGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQVYLGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGATGRAAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAAMVSGSAGYEGSTGDNGTWVTTTITSSNTGTAPHYYNYLFGME

SEQ ID NO: 9 - DNA, 1497 bp, synthetic construct
  misc_feature 1..1497: note = Description of Artificial Sequence: Synthetic polynucleotide
  misc_feature 1..1497: note = cDNA of AtPLT7
  source 1..1497: mol_type = other DNA
atggctcctccaatgacgaattgcttaacgttttctctgtcaccaatggagatgttgaaatcaactgatcagtctcacttctcttcttcttacgacgattcttctactccttatctcatcgataacttctatgctttcaaagaagaagctgagatagaagctgctgctgcttcaatggcggattcaacaaccttatctacttttttcgatcattctcagactcagattccaaagctggaagatttcctcggtgattcctttgtccgttactctgataaccaaacagagacccaagactcttcttctctcactccattctacgatccacgtcaccgcaccgttgccgaaggagttacagggttcttctctgatcatcatcagccagatttcaagacgataaactcgggaccagaaatcttcgatgactcaacaacttccaacatcggtggtactcatctctccagtcacgtggtggagtcatcaacgacggcgaagttagggtttaacggtgattgcaccaccaccggaggagttttgtctctaggggttaacaacacatcagatcaacctttgagctgtaacaatggcgagagaggtggaaacagtaacaagaagaaaacagtttctaagaaggaaacatcagatgattcaaagaagaagattgtcgaaacattgggacaaagaacttcaatttatcgtggagtcacccgacatagatggactggaagatacgaagcgcatctatgggataacagctgtaggagggaaggtcaagccagaaaaggacgtcaagtgtacttaggtggatatgacaaggaagatagagcagctagagcctatgacttggcagctttaaaatactggggttctactgctactacaaattttccggtctcgagttattcaaaagaacttgaggaaatgaatcacatgaccaagcaagagtttattgcatctcttaggaggaaaagtagcggtttttcgagaggagcttcaatatatagaggtgtcacaaggcatcatcaacaaggtcgctggcaagcaagaatcggccgtgtcgcaggaaacaaagatctttacctcggaacctttgcaaccgaagaggaagcagcagaggcttatgacattgcagccataaagttcagaggaatcaacgcagtaactaactttgagatgaacaggtatgacattgaagctgtcatgaatagttctttacctgtaggaggagcagctgcgaaacgccacaaactcaaactcgctcttgaatctccttcttcatcatcctctgaccataacctccaacaacaacagttgcttccgtcctcttctccctcggatcaaaaccctaactcaatcccatgtggcattccatttgagccttcagttctctattaccaccagaacttctttcagcattatcctttggtctctgactctacaattcaagctcctatgaaccaagctgagtttttcttgtggcctaaccagtcttactaa

SEQ ID NO: 10 - protein, 498 aa, Zea mays
MAPPMTNCLTFSLSPMEMLKSTDQSHFSSSYDDSSTPYLIDNFYAFKEEAEIEAAAASMADSTTLSTFFDHSQTQIPKLEDFLGDSFVRYSDNQTETQDSSSLTPFYDPRHRTVAEGVTGFFSDHHQPDFKTINSGPEIFDDSTTSNIGGTHLSSHVVESSTTAKLGFNGDCTTTGGVLSLGVNNTSDQPLSCNNGERGGNSNKKKTVSKKETSDDSKKKIVETLGQRTSIYRGVTRHRWTGRYEAHLWDNSCRREGQARKGRQVYLGGYDKEDRAARAYDLAALKYWGSTATTNFPVSSYSKELEEMNHMTKQEFIASLRRKSSGFSRGASIYRGVTRHHQQGRWQARIGRVAGNKDLYLGTFATEEEAAEAYDIAAIKFRGINAVTNFEMNRYDIEAVMNSSLPVGGAAAKRHKLKLALESPSSSSSDHNLQQQQLLPSSSPSDQNPNSIPCGIPFEPSVLYYHQNFFQHYPLVSDSTIQAPMNQAEFFLWPNQSY

SEQ ID NO: 11 - DNA, 1104 bp, synthetic construct
  misc_feature 1..1104: note = Description of Artificial Sequence: Synthetic polynucleotide
  misc_feature 1..1104: note = cDNA of TaRKD4
  source 1..1104: mol_type = other DNA
atggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttag

SEQ ID NO: 12 - protein, 367 aa, Triticum aestivum
MEMQQQYFGGDGDADWFHQLALLPPLPISSSLPPLPMSEGSCLPMAAAAAAALPLGDCSSALMIRPEEQMSCLPMNPSPAVVDDVYSSYAPNNVDVLPPFPAGLDDALLMESFSDIDLEEFADAFGHKIKTEPLDDAMVPADHDFAAQAQQACPVVIMNQQQLNAPRDVRLLIDPDDDDSTVVAGGYEAAAVGCAEQKQVRPAPRRVRKSSGGARPAAGGKSLDHIGFEELRTYFYMPITKAAREMNVGLTVLKKRCRELGVARWPHRKMKSLRSLILNIQEMGKGATSPAAVQGELEALERYCAIMEENPAIELTEQTKKLRQACFKENYKRRRAAASVNLLDHCYNDLASHEQQMPLPQMGFFGF

SEQ ID NO: 13 - DNA, 897 bp, synthetic construct
  misc_feature 1..897: note = Description of Artificial Sequence: Synthetic polynucleotide
  misc_feature 1..897: note = cDNA of AtRKD4
  source 1..897: mol_type = other DNA
atggctgatcacacaaccaaagaacagaagtcattctcattcctagctcattctccatcctttgatcacagctccttaagttatcctttattcgactgggaagaagatcttcttgctctccaagaaaactctggctctcaagcatttccttttactacaacttctctgcctttacctgatcttgaacccttgtctgaagatgtactcaattcatacagctctgcgtcatggaacgaaacagagcaaaacagaggagatggcgcttcatcggagaagaagagggaaaatggaacagtgaaagagacaactaagaagaggaaaatcaatgagagacacagagaacatagcgtgagaatcatcagcgatattactacctacacaactagttcagctccaacgacattgtcaaaggaaactgtctctcgctacttctacatgcccataactcaggctgcaatagcacttaacgttggtttaactctactaaaaaggagatgtcgcgaattgggtattcgccgatggcctcatcgtaaacttatgagcttaaacactttgatcagtaacgtcaaggagctgcagaagatggaaggcgaagagaatgcagaaaaactgcaggacgcgttggagatgcttgagaaggagaagaggacaattgaggatttgccggatttggagtttaaggacaagacaaagaggctaagacaagcttgtttcaaggctaaccacaagaggaagaagaagagaagtctcaagtccgatcagtctcaagtaccctcgtgttcaagcagcggatcagttcctagtgatgagtcggttgatgaagcaggaatggagagtgatgaagaaatgaagtatctcttgtgtggtttctcaagtgaatttactagtggtttgtga

SEQ ID NO: 14 - protein, 298 aa, Arabidopsis thaliana
MADHTTKEQKSFSFLAHSPSFDHSSLSYPLFDWEEDLLALQENSGSQAFPFTTTSLPLPDLEPLSEDVLNSYSSASWNETEQNRGDGASSEKKRENGTVKETTKKRKINERHREHSVRIISDITTYTTSSAPTTLSKETVSRYFYMPITQAAIALNVGLTLLKRRCRELGIRRWPHRKLMSLNTLISNVKELQKMEGEENAEKLQDALEMLEKEKRTIEDLPDLEFKDKTKRLRQACFKANHKRKKKRSLKSDQSQVPSCSSSGSVPSDESVDEAGMESDEEMKYLLCGFSSEFTSGL

SEQ ID NO: 15 - DNA, 1185 bp, synthetic construct
  misc_feature 1..1185: note = Description of Artificial Sequence: Synthetic polynucleotide
  misc_feature 1..1185: note = cDNA of ZmRKD4
  source 1..1185: mol_type = other DNA
atggcgatggtgccatgcggcgatgacaccgactggtgccacgtgctggacaacttcaacctgttgctgtgttcgtcgtcctgctcgccgaatgctatggccaacagagcggaagactgtctgccgatatctgctgctccacccggacccggccatcatcagagctgctgcaaaaacgaagtcgtcctcgaagcctcttgtgatggcgcgtttgctgcagccgactgcttgtcttcggctctgacgaacctgcagagggaggacgacagtttctatttgcccatgtactctgcgccacccgcagtcggcgatgagtacttctccgatctactcgcgcccgatgccgacggcattgacgaggcgctcctgatgccgttcagcgacatcgatcttcaggtcttcgacagtgacgacgagcacaggcctcctgtcgaccaaatggttaatatgatcccgccggcggttcttcatcatccctccaccgccgggacgcaaaatggaggtgccgttcatgctcatcagaaggccatggcggtcatcgatgactcctgtttccgacgaggagccagtggtgtcgagatggccgtcgtcaggcatcatggtgagcctcgtcaaggaagctcttccgtggcgccagtgccgccaccgtcactgccggggacgcgtgcaaggaggagcgacggccgatcagctcgggcggggaagacgacgaagctggactacatcggcttcgacgagctgcggaagtacttctgcatgcccatcaccagggcggcgagggagatgaacgtcgggctcaccgtgctcaagaagcgctgccgcgagctcggcgtggcgcggtggcctcaccggaagatgaagagcctcaagtccctcatggccaacgtccaggaaatggggaacgtcatgtcctcggtggctgtgcagcaggagcttgcggcgctcgagacgtactgcacgctcatggaggacaatccctggatcgagctcacggacaggaccaagaagctgcgccaggcgtgcttcaaggagaggtacaagcgtaggagggcggccgaagtcaacgtcatggatatggatcgcatctactgctttggccagcatcaccaccagcagctgctgcctccgacgacaagcagttctgacgaccgccatggccagtgcagccgttcctttggctactga

SEQ ID NO: 16 - protein, 394 aa, Zea mays
MAMVPCGDDTDWCHVLDNFNLLLCSSSCSPNAMANRAEDCLPISAAPPGPGHHQSCCKNEVVLEASCDGAFAAADCLSSALTNLQREDDSFYLPMYSAPPAVGDEYFSDLLAPDADGIDEALLMPFSDIDLQVFDSDDEHRPPVDQMVNMIPPAVLHHPSTAGTQNGGAVHAHQKAMAVIDDSCFRRGASGVEMAVVRHHGEPRQGSSSVAPVPPPSLPGTRARRSDGRSARAGKTTKLDYIGFDELRKYFCMPITRAAREMNVGLTVLKKRCRELGVARWPHRKMKSLKSLMANVQEMGNVMSSVAVQQELAALETYCTLMEDNPWIELTDRTKKLRQACFKERYKRRRAAEVNVMDMDRIYCFGQHHHQQLLPPTTSSSDDRHGQCSRSFGY

SEQ ID NO: 17 - DNA, 1125 bp, synthetic construct
  misc_feature 1..1125: note = Description of Artificial Sequence: Synthetic polynucleotide
  misc_feature 1..1125: note = cDNA of TaRKD2
  source 1..1125: mol_type = other DNA
atggagatgcagcagtacttcggcggctgcggcgatggcgatgctgactggttccatcagctcgccttgctcccgcctttgccggtctcttcgtctctgccgcctctccccatgagcgagggcagctgcttacctatggccgccgccgccccaacgcttcctcttggggattgctcatcagctctcatgattaggccggaagaacagatgggctgcctgcagatgatacctccacaggctgttgccgatgatgagtacagcagctacgccaccaacaatgtcgacgtcctcccgccgtttcctgcaggtctcgatgatcccacggcaggcctcgacgacgcgctgctcatggagtccttcagagacatcgacctggaggagttcgccgacgccgtcggccccaagattaagaccgagcctctcgacgacgccatggtgccggcggatcacgatttcgcggcgcaagtgcaacaggcgcgccccgtggtgatcatgaaccagcagcagctgaatgcgccacacggcgtgcgcctgctcaatgatcccgacgacgatgactcagctgtcgtcgccgggggctatgaggcggcggccgttgggtgcgctgagcagaagcgggtgaggccggcgccacgtcgtgtgcggaagagcagcggtgggtcacgccctgccgccggtgggaaaagcctcgatcacatagggtttgaggagctgcgtacgtatttctacatgcctatcaccaaggcggcgcgggagatgaacgtcggtctcaccgtgctcaagaagcgctgccgtgagctcggtgtcgcccgttggcctcaccggaagatgaagagcctcaggtctctcatccttaacatccaggacatggggaagggcgccacgtcgccggcggcggtgcaaggggagctggaggcgcttgagaggtattgtgccataatggaggagaacccggcgatcgagctgacggagcagaccaagaagctgaggcaggcctgctttaaggagaactacaagaggaggagagcggcggcctccgtcaacttgctcgagcattgctacaacgacttgggcagtcatgagcagcagatgccattgccacagatgggtttctttgggttctaa

SEQ ID NO: 18 - protein, 374 aa, Triticum aestivum
MEMQQYFGGCGDGDADWFHQLALLPPLPVSSSLPPLPMSEGSCLPMAAAAPTLPLGDCSSALMIRPEEQMGCLQMIPPQAVADDEYSSYATNNVDVLPPFPAGLDDPTAGLDDALLMESFRDIDLEEFADAVGPKIKTEPLDDAMVPADHDFAAQVQQARPVVIMNQQQLNAPHGVRLLNDPDDDDSAVVAGGYEAAAVGCAEQKRVRPAPRRVRKSSGGSRPAAGGKSLDHIGFEELRTYFYMPITKAAREMNVGLTVLKKRCRELGVARWPHRKMKSLRSLILNIQDMGKGATSPAAVQGELEALERYCAIMEENPAIELTEQTKKLRQACFKENYKRRRAAASVNLLEHCYNDLGSHEQQMPLPQMGFFGF

SEQ ID NO: 19 - DNA, 771 bp, synthetic construct
  misc_feature 1..771: note = Description of Artificial Sequence: Synthetic polynucleotide
  misc_feature 1..771: note = cDNA of AtRKD2
  source 1..771: mol_type = other DNA
atgagttcgtcaaaacattcctctgtttttaactattctgctctgtttctatcactgtttcttcaacaaatggatcagaactctcttcatcatctcgattctccaaaaatcgaaaacgagtatgaaccagattcgttatacgacatgttagataagttgcctccgcttgattctctcctagatatggaagatttgaaaccaaatgcagggttgcactttcagttccattacaatagctttgaagatttcttcgaaaacattgaagtggataacacaattccatctgatattcacttgttgacacaagagccctacttctcaagtgactcctcttcctcttcaccattggctatccaaaacgacggtctcatttccaacgtgaaagttgaaaaggtaacagttaagaagaagaggaaccttaagaaaaagaggcaagacaaattggagatgtctgagatcaaacaatttttcgataggccgatcatgaaagcggctaaagaactgaacgtgggactcactgtgttgaagaagcgatgcagggaattaggaatttaccggtggcctcaccggaagctcaagagtctaaactctcttataaagaatctcaagaatgttggaatggaagaggaagtgaagaacttggaggaacataggtttcttattgaacaagaacctgatgcagaactcagtgatggaaccaagaagctaaggcaagcttgtttcaaagccaattataagagaagaaaatcacttggtgatgattattattga

SEQ ID NO: 20 - protein, 256 aa, Arabidopsis thaliana
MSSSKHSSVFNYSALFLSLFLQQMDQNSLHHLDSPKIENEYEPDSLYDMLDKLPPLDSLLDMEDLKPNAGLHFQFHYNSFEDFFENIEVDNTIPSDIHLLTQEPYFSSDSSSSSPLAIQNDGLISNVKVEKVTVKKKRNLKKKRQDKLEMSEIKQFFDRPIMKAAKELNVGLTVLKKRCRELGIYRWPHRKLKSLNSLIKNLKNVGMEEEVKNLEEHRFLIEQEPDAELSDGTKKLRQACFKANYKRRKSLGDDYY

SEQ ID NO: 21 - DNA, 936 bp, synthetic construct
  misc_feature 1..936: note = Description of Artificial Sequence: Synthetic polynucleotide
  misc_feature 1..936: note = cDNA of ZmRKD2
  source 1..936: mol_type = other DNA
atgacgggcctcgacgaggcgctcatgctgccgttcaccgacatcgatcttgaggccttcgacaacgccgaagagcaaaagcctcctgtcgaccaaatggttatgatgccgccgacggttgaacaccccgccgccgccgggacgcgagccccaatcatcattgatggtacggcgaccgttggccaaaatgtaggtggtggtgtcgtccacgctcatcagaaggcggccatgacgaccatagaggactccagctgcttccgacgaggagccagctgtgtcgacgacgacatggccgtcgtcattcaccatgtcgagcgtcgtcgtcaagcaggctctaccgccgtggcgctattgccgccgccgcagccgtcactgccgcggccgcgtgcaagggcgagcggcggcgcgggcgagcggtcagctccggcggccgccgggaagacgaggatggaccacatcggcttcgacgagctgcgcaagtacttctacatgcccatcaccagggcggccagggagatgaacgtggggctcaccgtgctcaagaagcgctgccgcgagctcggcgtggcgcggtggcctcaccggaagatgaagagcctcaagtccctcatggccaacgtacaggaaatggggaacggcatgtcgccggtggctgtgcagcatgagcttgcggcgctggagacgtactgcgcgctcatggaggagaacccatggatcgagctcacggaccggacgaagaggctgcggcaggcctgcttcaaggagagctacaagcggaggaaggcggccgcaggcaacgctatcgagacggatcacattgtctacagctttggacagcatcgtcgttacaagcagcagctgctgcctccgccaactgcgggtagtaccagtgctgacgaccgccatggccagagcagccgtttcttttgctactga

SEQ ID NO: 22 - protein, 311 aa, Zea mays
MTGLDEALMLPFTDIDLEAFDNAEEQKPPVDQMVMMPPTVEHPAAAGTRAPIIIDGTATVGQNVGGGVVHAHQKAAMTTIEDSSCFRRGASCVDDDMAVVIHHVERRRQAGSTAVALLPPPQPSLPRPRARASGGAGERSAPAAAGKTRMDHIGFDELRKYFYMPITRAAREMNVGLTVLKKRCRELGVARWPHRKMKSLKSLMANVQEMGNGMSPVAVQHELAALETYCALMEENPWIELTDRTKRLRQACFKESYKRRKAAAGNAIETDHIVYSFGQHRRYKQQLLPPPTAGSTSADDRHGQSSRFFCY

SEQ ID NO: 23 - DNA, 1883 bp, Brachypodium distachyon
  source 1..1883: mol_type = unassigned DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagttt

SEQ ID NO: 24 - DNA, 5100 bp, synthetic construct
  misc_feature 1..5100: note = Description of Artificial Sequence: Synthetic polynucleotide
  misc_feature 1..5100: note = pABM-BdEF1
  source 1..5100: mol_type = other DNA
gatcccccgggctgcaggaattcaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttg

SEQ ID NO: 25 - DNA, 6567 bp, synthetic construct
  misc_feature 1..6567: note = Description of Artificial Sequence: Synthetic polynucleotide
  misc_feature 1..6567: note = pABM-BdEF1_ZmPLT5
  source 1..6567: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaa

SEQ ID NO: 26 - DNA, 6552 bp, synthetic construct
  misc_feature 1..6552: note = Description of Artificial Sequence: Synthetic polynucleotide
  misc_feature 1..6552: note = pABM-BdEF1_ZmPLT7
  source 1..6552: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtaga

SEQ ID NO: 27 - DNA, 5865 bp, synthetic construct
  misc_feature 1..5865: note = Description of Artificial Sequence: Synthetic polynucleotide
  misc_feature 1..5865: note = pABM-BdEF1_KWS_RBP1
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaa

SEQ ID NO: 28 - DNA, 6186 bp, synthetic construct
  misc_feature 1..6186: note = Description of Artificial Sequence: Synthetic polynucleotide
  misc_feature 1..6186: note = pABM-BdEF1_TaRKD4
  source 1..6186: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttaga

SEQ ID NO: 29 - DNA, 10427 bp, synthetic construct
  misc_feature 1..10427: note = Description of Artificial Sequence: Synthetic polynucleotide
  misc_feature 1..10427: note = pGEP359
  source 1..10427: mol_type = other DNA
actgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcccgggatatcgcggccgcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcacgtctaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacatgatcagggagctctcaggtacctctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggaggacgagcggcgtaccttgaagcggaggtgccgacgggtggatttgggggagatccactagttctagagcggccgccaccgcggtggaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgaccgatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaattcacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatcaagtgacagatagctgggcaatggaatccgaggaggtttcccgatattaccctttgttgaaaagtctcaatagccctttggtcttctgagacttgcaggcaagcaagcatgaatgcctgggcgcgccgatatc

SEQ ID NO: 30 - DNA, 3841 bp, synthetic construct
  misc_feature 1..3841: note = Description of Artificial Sequence: Synthetic polynucleotide
  misc_feature 1..3841: note = pGEP324
  source 1..3841: mol_type = other DNA
ctgacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctcgtcacgattcccctctcctggggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcgtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 31 - DNA, 5611 bp, synthetic construct
  misc_feature 1..5611: note = Description of Artificial Sequence: Synthetic polynucleotide
  misc_feature 1..5611: note = pAMK-ZmWUS2-tDT-nosT
  source 1..5611: mol_type = other DNA
aggccttgaagacaaatccactagtcctgcaggggatcccttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttattaatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctccaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaattgcaaagacaagcgaatcatcagcacaaaaggtaaacaggaacacactgcaaagggtagtacaaaactcataaccatgtatgccttacattcgatgttccataaaaaaattaagtcttaatagcatcacggtttcaacgaaagtaataatacttcatgaccaggcaaacattgccatcatagattacttgttcacgcgacaactgcaaggatgtcaacaagacgagatattttaagcttccacgaggtaaccaacaagcaagcacagcaccagacagatagaagatccaatgcattggtcctgcaggccccgggctatctttgtcttccggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggccctta

SEQ ID NO: 32 - DNA, 3663 bp, synthetic construct
  misc_feature 1..3663: note = Description of Artificial Sequence: Synthetic polynucleotide
  misc_feature 1..3663: note = BdEF1::ZmPLT5_expression_cassette
  source 1..3663: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacacctcgcaccactatcatccatggctcaacttctccctcgcccaccactgtgacctcgaggaggaggagaggggcgcggccgccgagctggccgcgatagccggcgccgcgccgccgccgaagctggaggacttcctcggcggaggcgtcgccaccggtggtccggaggcggtggcgcccgcggagatgtacgactcggacctcaagttcatagccgccgccgggttccttggcggctcggcggcggcggcggcgacgtcgccgctgtcctccctcgaccaggccggttccaagctggccttgcctgcggcggcggctgctccggcgccggagcagaggaaggccgtcgactcctttgggcagcgcacgtccatctaccgcggcgtcacacggcaccggtggactggcaggtacgaggcacatctgtgggacaacagctgccgacgcgaagggcagagccgcaagggccgccaagtatatttgggtggctatgataaggaggagaaggctgccagggcgtatgatcttgcagctttgaagtactggggttctagcaccaccaccaactttccggttgctgagtatgagaaggaggtcgaggagatgaagaacatgacgcgacaagagtttgttgcttcccttcgaaggaagagcagtggattctctcggggtgcttccatctacagaggtgtaaccagacatcaccagcatggacggtggcaggcgaggatcggaagggtggccggtaacaaggacctctaccttgggacgttcagcaccgaggaggaagctgcagaggcctacgacatagcggccatcaagttcagaggcctgaacgccgtcacaaacttcgagatcagccggtacaacgtggagaccataatgagcagcaaccttccagtcgcgagcatgtcgtcgtcgtcggcggcggcggcgggtggccggagcagcaaggcgctggagtcccctccgtccggctcgcttgacggcggcggcggcatgccagtcgtcgaaggcagcacggcaccgccgctgttcattccggtgaagtacgaccagcagcagcaggagtacctgtcgatgctcgcgttgcagcaccaccaccagcagcaacaagcagggaacctgttgcaggggccgctagtagggttcggcggcctctactcctccggggtgaacctggatttcgccaactcccacggcacggcggctccgtcgtcgatggcccaccactgctacgccaatggcaccgcgtccgcctcgcatgagcaccagcaccagcaccagatgcagcagggcggcgagaacgagacgcagccgcagccgcagcagagctccagcagctgctcctccctgccattcgccaccccggtcgctttcaatgggtcctatgaaagctccatcacggcggcaggcccctttggatactcctacccaaatgtggcagcctttcagacgccgatctatggaatggaatgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 33 - DNA, 3648 bp, synthetic construct
  misc_feature 1..3648: note = Description of Artificial Sequence: Synthetic polynucleotide
  misc_feature 1..3648: note = BdEF1::ZmPLT7_expression_cassette
  source 1..3648: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggacatggacatgagctcagcttatccccaccattggctctccttctccctctccaacaactaccaccatggcctactcgaggccttctctaactcctccggtactcctcttggagacgagccgggcgcagtggaggagtccccgaggacggtggaggacttcctcggcggcgtcggtggcgccggcgccccgccgcagccggcggctgctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggcgggggcggagtccgaccaggcgaggcggcccgccgagacgttcggccagcgcacatccatctaccgtggcgtcaccaggcaccggtggacagggagatatgaggcgcacttgtgggacaacagctgccgccgggagggccaaagccgcaaaggacgccaagtctacctaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaacttcgacatgagccgctacgacgtggagagcatcctcagcagcgacctccccgtcgggggcggagccaccgggcgcgccgccaagttcccgttggactcgctgcagccggggagcgctgctgcgatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctgctatggtttctggatctgctggctacgagggctccaccggcgacaatggaacctgggttactacgactattaccagcagcaacaccggcacggctccccactactacaactatctcttcgggatggagtagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 34 - DNA, 2961 bp, synthetic construct
  misc_feature 1..2961: note = Description of Artificial Sequence: Synthetic polynucleotide
  misc_feature 1..2961: note = BdEF1::KWS_RBP1_expression_cassette
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagtcgggctccgggacggctgctggctctggctatgtttacagacagccaggatcaacgcggtggaacccgacagctgaacaactgtccttgcttagagaaatctactaccgcaacggattgcggaccccgaccgcggacgaaatcagacaaatcagctcaaagctctcaaggtacggaaaaatagagggcaaaaacgtttacaactggttccagaatagacgcgcaagagaaaagcgcaagcaacggctctctacaatcggctgtgatccagcactgatcgagatggggaatgtcgcttcactggaattcggtactgagagcgccctggaatcgctgtcgtcaggaccatcctcagaactccgcgaagcgccaacgagaaaattttacgaaaaaaagacggttggagagaactcaactataataaacccagtggaacaaaactgtaccctttcctgcggaacgtcccaagagttccagtatgcggtcgattctcggcgcgtcatgaaagctatggaggaaaagcaggcgacggacgatgaacccgacggaaataaatggactgagtcaaacagacacgtcaagattctccagcttttcccgctccacaataacgaggatcagacattgataaagagcgacaaagaaatctattgtttgggctcgtgcgagaagaaaatggatttgtcaccgctgggtcattcaggctctcagcgcgcttcggcccttgacttgtgcctttcattgggcaacgaatcttgtgggctgcatgataattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 35 - DNA, 3282 bp, synthetic construct
  misc_feature 1..3282: note = Description of Artificial Sequence: Synthetic polynucleotide
  misc_feature 1..3282: note = BdEF1::TaRKD4_expression_cassette
  source 1..3282: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggagatgcaacaacaatacttcgggggggacggcgatgcggactggttccatcaactcgcattgcttcccccacttccaatctcatcgtctctccccccactcccgatgtcagagggctcatgtctccctatggcagcagcagctgcagctgcactcccccttggcgattgctcgagcgccctcatgatacgccctgaggaacagatgtcttgccttccaatgaacccctctccagcggtcgtcgacgatgtctactcttcctacgcaccgaacaatgtcgacgtgttgccgccattcccggcaggacttgacgacgctctgttgatggagtctttttctgacatcgacctcgaggagtttgctgacgcatttggccacaagatcaagacagaacccctcgacgatgccatggtccccgcggaccacgacttcgcggctcaagcccaacaggcctgccctgtggtcatcatgaatcagcaacaactcaacgcacccagagacgtgcgcctgctcattgacccggatgatgatgacagcaccgtggtggccgggggctatgaagctgcagcggtggggtgcgccgagcagaaacaggtcaggccagcaccacgtagggtgagaaagagctcaggcggcgcaagaccagccgcgggaggaaagtccctcgatcacatcggattcgaggaactcaggacctatttctatatgccaatcaccaaggcagcgagggaaatgaacgtggggctgacagtcctgaagaagagatgccgggaactgggggtggcgcgctggccacacagaaagatgaagtctctgagaagcctgatcctcaacattcaggagatggggaagggcgcaacatctcccgcagccgtgcagggggaacttgaagcgcttgagaggtattgcgccattatggaggagaacccggctatagagctcaccgagcaaacgaagaagctcaggcaggcttgtttcaaagagaattataagcggcgtagagccgccgcttctgttaatcttctcgatcactgctataacgatctggcatctcatgagcagcaaatgcctctcccacaaatgggattctttggattttagaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 36 - DNA, 3080 bp, synthetic construct
  misc_feature 1..3080: note = Description of Artificial Sequence: Synthetic polynucleotide
  misc_feature 1..3080: note = BdEF1::ZmWUS2_expression_cassette
  source 1..3080: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttattaatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctccaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgaattgcaaagacaagcgaatcatcagcacaaaaggtaaacaggaacacactgcaaagggtagtacaaaactcataaccatgtatgccttacattcgatgttccataaaaaaattaagtcttaatagcatcacggtttcaacgaaagtaataatacttcatgaccaggcaaacattgccatcatagattacttgttcacgcgacaactgcaaggatgtcaacaagacgagatattttaagcttccacgaggtaaccaacaagcaagcacagcaccagacagataga

SEQ ID NO: 37 - DNA, 5561 bp, synthetic construct
  misc_feature 1..5561: note = Description of Artificial Sequence: Synthetic polynucleotide
  misc_feature 1..5561: note = pUbi::LpCpf1_expression_cassette
  source 1..5561: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaagcttgaagcaaacatggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtgagtcgaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatc

SEQ ID NO: 38 - DNA, 1945 bp, synthetic construct
  misc_feature 1..1945: note = Description of Artificial Sequence: Synthetic polynucleotide
  misc_feature 1..1945: note = pUbi::crRNA5_expression_cassette
  source 1..1945: mol_type = other DNA
gacgcgccctgtagcggcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccaaattactgatgagtccgtgaggacgaaacgagtaagctcgtctaatttctactaagtgtagatctcgtcacgattcccctctcctggggccggcatggtcccagcctcctcgctggcgccggctgggcaacatgcttcggcatggcgaatgggaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatc

SEQ ID NO: 39 - DNA, 3771 bp, synthetic construct
  misc_feature 1..3771: note = Description of Artificial Sequence: Synthetic polynucleotide
  misc_feature 1..3771: note = codon-optimized LbCpf1
  source 1..3771: mol_type = other DNA
atggcatctagcatggcaccaaagaaaaaaaggaaagtttccaaacttgaaaaatttacaaactgctactccctttccaagacgcttaggtttaaagcgatccccgttggcaagacccaagagaatatcgataacaaaagacttctggtcgaagatgaaaaaagggccgaagactacaagggggtcaagaagttgctcgatcgctattatctttcctttatcaacgatgtgcttcattcaatcaaactgaagaacttgaataactacattagccttttcagaaagaaaacgaggactgaaaaggagaacaaggaacttgagaatcttgaaataaaccttcgcaaagaaattgcaaaagccttcaaggggaacgaaggatataaatctcttttcaaaaaagacattatagaaacaattttgcctgagtttcttgacgacaaggatgaaattgcgctcgtcaatagctttaacggatttacaactgccttcacagggttcttcgacaatagggagaatatgtttagcgaggaggcaaaaagcacatccatcgcattcagatgcatcaatgaaaatcttacccggtacatatcgaatatggacatatttgaaaaagtggatgcaatattcgataagcacgaagtccaggagataaaggaaaagatactgaatagcgactatgatgtcgaagattttttcgaaggtgagttcttcaactttgtcctgactcaagaaggcattgatgtctataatgcaataattggaggttttgtgactgagtctggcgagaagataaagggcttgaacgagtatatcaatctctacaaccagaagactaagcaaaagttgcctaaatttaaaccgctttacaagcaagttttgagcgaccgggaaagcctttccttttacggtgaaggatacacgagcgatgaagaagtcctcgaagtcttccgcaacacactcaacaagaactcagaaatcttttcctcaattaaaaaattggagaagcttttcaagaacttcgatgaatactcttcggcggggatttttgtgaagaacggcccggcaatttccacaatatctaaagacattttcggagaatggaacgtgataagagacaagtggaatgcggagtatgatgacatacacctgaagaagaaggcagttgtgactgaaaaatacgaagatgacaggagaaaaagctttaaaaagatcgggtccttttcactggaacagctgcaggagtatgccgacgccgatctttcggttgtcgaaaagctcaaagaaataattatccagaaggtcgatgaaatctacaaggtgtacggctcaagcgagaagctctttgatgctgacttcgtgttggagaagtctcttaaaaaaaacgacgcagtcgtcgcgataatgaaagatttgctggattcagtgaaatccttcgagaattatatcaaagccttcttcggcgaggggaaggagacaaacagggatgagtccttctatggagacttcgttctggcttacgacatccttcttaaggtcgaccacatctatgacgcaattcggaactatgtgacgcagaagccgtattcgaaagataagttcaagctctatttccaaaaccctcaatttatgggtgggtgggataaagacaaagagaccgattaccgggcaacaattttgcggtacgggtctaaatattacctcgctataatggataagaaatacgctaaatgtctccagaaaattgacaaagatgacgtcaacggcaattatgaaaaaatcaattataaactccttcctggcccaaataaaatgctcccgaaggtgtttttttccaaaaagtggatggcctattataatccatcagaggatattcagaaaatctataaaaatgggacctttaagaagggtgacatgtttaacctgaacgattgccacaagcttatagattttttcaaagactctattagccgctatcccaaatggtctaatgcttatgatttcaacttctctgaaactgaaaagtacaaagatattgcaggattctaccgcgaagttgaagaacaaggttataaggtttcctttgagtctgcgtccaagaaagaggtcgataagttggtcgaagaagggaaattgtatatgtttcaaatttacaataaagacttttccgacaagtcccatggtacacctaatctgcataccatgtacttcaaactgctgttcgatgagaataatcacggtcagattcgcctgagcggaggggcggaactcttcatgaggagagcatcgttgaaaaaagaggagctcgtcgtgcatccggctaacagccccattgctaacaagaatccggataatccaaagaagactactaccctctcctatgacgtctataaggataagagattctctgaggaccagtacgagttgcacatccctattgcgataaataaatgccctaagaacatctttaaaatcaatactgaggtcagagtcctgcttaagcacgacgacaacccgtatgtgatcgggattgataggggtgaaaggaacttgctttatattgtggttgtcgatggaaaaggtaatatagtggaacaatactctctgaatgaaattatcaacaacttcaatggcattaggatcaagaccgactatcattctctgttggacaagaaagagaaagagcgcttcgaggcacggcaaaactggacgtctattgagaacatcaaggagcttaaggctggttacatttctcaggttgtgcacaaaatttgcgaactggtcgagaaatatgatgccgttatcgcacttgaagatctcaacagcggatttaagaattctcgggtgaaagtcgaaaaacaggtgtatcaaaaattcgaaaagatgctgatcgacaagctcaattatatggttgataaaaagagcaacccatgcgccacggggggtgcgcttaagggctatcagattacgaacaaatttgaatccttcaagtcaatgtcgacgcaaaatgggtttatattctatataccggcgtggcttacatctaaaatagatcctagcactgggttcgtgaacctgctgaaaaccaagtacacttcaatcgcagattctaaaaaatttataagcagcttcgacagaatcatgtatgtgcccgaggaagacctcttcgagtttgcccttgattacaaaaatttctcaagaacggatgcagactacataaagaagtggaagctgtactcttatgggaaccggattcggatattcagaaatccgaaaaaaaacaatgtctttgattgggaggaagtttgtcttacctctgcttacaaagagctgttcaataaatatggcattaattaccagcaaggtgatatccgggcgctcctttgcgaacagtctgacaaagctttctattcttcatttatggcgctcatgtcattgatgctgcagatgaggaatagcattacggggaggactgatgttgactttctgatctcgcccgtgaaaaattctgatggaatcttctacgattccaggaattatgaggcccaggaaaatgctatccttcccaagaacgcagacgcaaatggcgcgtacaatatagctcgcaaggttttgtgggctataggccaattcaagaaagccgaagacgaaaagctggacaaagttaagattgctatatctaacaaagagtggcttgagtatgcgcaaacatctgttaaacacaaacgccccgcggctacaaagaaggctggccaggcaaagaagaagaagtga

SEQ ID NO: 40 - protein, 1256 aa, unidentified
  REGION 1..1256: note = Description of Unknown: Lachnospiraceae bacterium sequence
MASSMAPKKKRKVSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHKRPAATKKAGQAKKKK

SEQ ID NO: 41 - DNA, 201 bp, synthetic construct
  misc_feature 1..201: note = Description of Artificial Sequence: Synthetic polynucleotide
  misc_feature 1..201: note = crRNA5_target_HMG13_referrence_A188
  source 1..201: mol_type = other DNA
gaaccctgagagctgctttatgaccggccccatattattactatctactttgacttttcccttaatgacgacttattatttgatttactcgtcacgattcccctctcctggtcgaacttttcaggtggggaaagctgctggcgacaggtggaaatccctgagcgagtcggtaagctccatcttctgtactaaagtagtagt

SEQ ID NO: 42 - DNA, 21 bp, synthetic construct
  misc_feature 1..21: note = Description of Artificial Sequence: Synthetic oligonucleotide
  misc_feature 1..21: note = crRNA5_target_sequence
  source 1..21: mol_type = other DNA
taatttctactaagtgtagat

SEQ ID NO: 43 - DNA, 4610 bp, synthetic construct
  misc_feature 1..4610: note = Description of Artificial Sequence: Synthetic polynucleotide
  misc_feature 1..4610: note = construct of pAMK-ZmWus2-tDT-nosT
  source 1..4610: mol_type = other DNA
aggccttgaagacaaatccactagtggatcccgagatttccatcgcacaagacacgaaaaaatcccgatcaatttaacgaacattgttttgcattatagattatattgtttacagaatgaagttaactaaaaccttaaccttttgcagataaatctctaaatagtgccgtactgtatacactcgagatttccaccgcacaagacatgagaaaattccggtcgatttgacaaagactgggtgttattaattagaggaagcagatccagccacatgttgtctcacatctgatcccccacgtatagtcgtatacgtttggcccaaacctagctcgatccatgtatgaaacacgtctcgtctcgccttctacctcctttttctatcacaggagattaaagtgagagagagagggcgctcaatgaactgcggcattgaacaatggagctgcaagagcaatgatgcactagctagtgtaatgcagtgcatgcatggtagattggtagcttgcctttgcagtttgcaccaggcaccagcagcagctagaagacgacagacgacaggggtttggctgctaggttgcggaagggcagttaccagttgccacaaggggagcctggccctctgcatcctcctcatgatagctctgtctctctctctcacagacacacacacagagactcttccaaattccgaagcggccaatgcaatgcaagagccagcccccggccgtgtgtcaacttcacttgtctctctccaaaagatatcgtatcacccatggccatgacccccctcccccagccccaacctatatcacctagcgcagctacgctctcttctcccgctctcgctctctgcatgctagctaccttctagctatctagcctctaggtccaatgcactccctccttataaacaaggaaccctccttcgcctctcttgccatagaccggacaccggagaggtcactgcacaggagcgctcaggaaggccgctgcgctgagatagaggcattatctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaaatgccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgaagatcccccgggctatctttgtcttccggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcaccggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggccctta

SEQ ID NO: 44 - DNA, 1458 bp, Zea mays
  source 1..1458: mol_type = unassigned DNA
atggacatggacatgagctcagcttatccccaccattggctctccttctccctctccaacaactaccaccatggcctactcgaagccttctctaactcctccggtactcctcttggagacgagcagggcgcagtggaggagtccccgaggacggtggaggacttcctcggcggcgtcggtggcgccggcgccccgccgcagccggcggcggctgcagatcaggatcaccagcttgtgtgcggcgagctgggcagcatcacagccaggttcttgcgccactacccggcggcgccagctgggacgacggtggagaaccccggcgcggtgaccgtggcggccatgtcgtcgacggacgtggccggggcggagtccgaccaggcgaggcggcccgccgagacgttcggccagcgcacatccatctaccgtggcgtcaccaggcaccggtggacggggagatatgaggcgcacctgtgggacaacagctgccgccgggagggccaaagccgcaaaggacggcaaggaggctatgacaaggaggagaaggcggctagagcttacgacctcgccgcgctcaagtactgggggcctacaaccacgaccaacttcccggtgtccaactacgagaaggagctggaggagatgaagtccatgacgcggcaggagttcatcgcgtcgttgcgcaggaagagcagcggcttctcacgaggcgcctccatctacagaggagtcacaaggcatcatcagcacggccggtggcaggcgaggatcggcagggtggccggaaacaaggacctgtacttgggcactttcagtactcaggaagaggcggcggaggcgtacgacatcgctgcgatcaagttccgcgggctcaacgccgtcaccaactttgacatgagccgctacgacgtggagagcatcctcagcagcgacctccccgtcgggggcggagctagcggtcgcgcccccgccaagttcccgttggactcgctgcagccggggagcgctgccgccatgatgctcgccggggctgctgccgcttcgcaggccaccatgccgccgtccgagaaggactactggtctctgctcgccctgcactaccagcagcagcaggagcaggagcggcagttcccggcttctgcttacgaggcttacggctccggcggcgtgaacgtggacttcacgatgggcaccagtagcggcaacaacaacaacaacaccggcagcggcgtcatgtggggcgccaccactggtgcagtagtagtgggacagcaagacagcagcggcaagcagggcaacggctatgccagcaacattccttatgctgctgctgctatggtttctggatctgctggctacgagggctccaccggcgacaatggaacctgggttactacgactaccagcagcaacaccggcacggctccccactactacaactatctcttcgggatggagtag

SEQ ID NO: 45 - protein, 485 aa, Zea mays
MDMDMSSAYPHHWLSFSLSNNYHHGLLEAFSNSSGTPLGDEQGAVEESPRTVEDFLGGVGGAGAPPQPAAAADQDHQLVCGELGSITARFLRHYPAAPAGTTVENPGAVTVAAMSSTDVAGAESDQARRPAETFGQRTSIYRGVTRHRWTGRYEAHLWDNSCRREGQSRKGRQGGYDKEEKAARAYDLAALKYWGPTTTTNFPVSNYEKELEEMKSMTRQEFIASLRRKSSGFSRGASIYRGVTRHHQHGRWQARIGRVAGNKDLYLGTFSTQEEAAEAYDIAAIKFRGLNAVTNFDMSRYDVESILSSDLPVGGGASGRAPAKFPLDSLQPGSAAAMMLAGAAAASQATMPPSEKDYWSLLALHYQQQQEQERQFPASAYEAYGSGGVNVDFTMGTSSGNNNNNTGSGVMWGATTGAVVVGQQDSSGKQGNGYASNIPYAAAAMVSGSAGYEGSTGDNGTWVTTTTSSNTGTAPHYYNYLFGME

SEQ ID NO: 46 - DNA, 2107 bp, synthetic construct
  misc_feature 1..2107: note = Description of Artificial Sequence: Synthetic polynucleotide
  misc_feature 1..2107: note = pZmWUS2::tDT-nosT_expression_cassette
  source 1..2107: mol_type = other DNA
cgagatttccatcgcacaagacacgaaaaaatcccgatcaatttaacgaacattgttttgcattatagattatattgtttacagaatgaagttaactaaaaccttaaccttttgcagataaatctctaaatagtgccgtactgtatacactcgagatttccaccgcacaagacatgagaaaattccggtcgatttgacaaagactgggtgttattaattagaggaagcagatccagccacatgttgtctcacatctgatcccccacgtatagtcgtatacgtttggcccaaacctagctcgatccatgtatgaaacacgtctcgtctcgccttctacctcctttttctatcacaggagattaaagtgagagagagagggcgctcaatgaactgcggcattgaacaatggagctgcaagagcaatgatgcactagctagtgtaatgcagtgcatgcatggtagattggtagcttgcctttgcagtttgcaccaggcaccagcagcagctagaagacgacagacgacaggggtttggctgctaggttgcggaagggcagttaccagttgccacaaggggagcctggccctctgcatcctcctcatgatagctctgtctctctctctcacagacacacacacagagactcttccaaattccgaagcggccaatgcaatgcaagagccagcccccggccgtgtgtcaacttcacttgtctctctccaaaagatatcgtatcacccatggccatgacccccctcccccagccccaacctatatcacctagcgcagctacgctctcttctcccgctctcgctctctgcatgctagctaccttctagctatctagcctctaggtccaatgcactccctccttataaacaaggaaccctccttcgcctctcttgccatagaccggacaccggagaggtcactgcacaggagcgctcaggaaggccgctgcgctgagatagaggcattatctcaacacaacatatacaaaacaaacgaatctcaagcaatcaagcattctacttctattgcagcaatttaaatcatttcttttaaagcaaaagcaattttctgaaaattttcaccatttacgaacgatagggcgcgatcccgccaccatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtaaatgccgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 47 - DNA, 783 bp, synthetic construct
  misc_feature 1..783: note = Description of Artificial Sequence: Synthetic polynucleotide
  misc_feature 1..783: note = cDNA of KWS-RBP2
  source 1..783: mol_type = other DNA
atggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattga

SEQ ID NO: 48 - protein, 260 aa, synthetic construct
  REGION 1..260: note = Description of Artificial Sequence: Synthetic polypeptide
  REGION 1..260: note = protein of KWS-RBP2
MESGSGTAAGSGYVYRQSGSTRWNPTAEQLSLLKELYYRNGIRTPSADQIRQISARLSRYGKIEGKNVFYWFQNHKARERQKKRLSTVGCDPALIEMGNVASLEFGTESALESLSSGPSSELREAPTRKFYEKKTVGENSTIINPVEQNCTLSCGTSQEFQYAVDSRRVMKAMEEKQATDDEPDGNKWTESNRHVKTLPLFPLHNNEDQTLIKSDKEIYCLGSCEKKMDLSPLGHSGSQRASALDLCLSLGNESCGLHDN

SEQ ID NO: 49 - DNA, 5865 bp, synthetic construct
  misc_feature 1..5865: note = Description of Artificial Sequence: Synthetic polynucleotide
  misc_feature 1..5865: note = pABM-BdEF1_KWS-RBP2
  source 1..5865: mol_type = other DNA
agcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttactagacttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaa

SEQ ID NO: 50 - DNA, 2961 bp, synthetic construct
  misc_feature 1..2961: note = Description of Artificial Sequence: Synthetic polynucleotide
  misc_feature 1..2961: note = BdEF1::KWS-RBP2_expression_cassette
  source 1..2961: mol_type = other DNA
cttcaccgccattgcaaaaattgtcaataaatatttagagtgggtggcatcagaaaaacatctctagtggactctcttcctatcatagctactcgggctgtagatagaacgagggcacaagagttgggtggcgtaggtttactcgtgacctcaactcttttggctgtgtcttacgtctaagatgggtttggcatgtgagaaacataggtctaagcaattcatgttagggctgttgcattgttgttgcatcaaccaaatgtccagatagcagttcatgctacatctagttgaaaaccctcatcattaggcggaacatgtgttcttttttagcatagtcaaagtcagattgcggcactcgctcatccacggaaagaattttccctgtgcaggcatctcgatcaaaagacgcaaattaatttttgaatagcgatataacaatatctaattaacgtttcttgttttctgcgaaatgtctttcatcataaaatgagtcatctcgatgagcccaagtgacatagcccaacaccccaccccaccaataaaagtgaagaaaacatgttgggaaaactataccaagtaaaatacgagttgttctaaagaaaaagtaaagtacgagttagatcgcaccctgtcctggagtgtggcttgatgatccaactcctagcattgtatccctgtttttggatgatgtaactattatttacaatgaataaagaggtgttttactagtaaaaaaatcttgaggggaggagaaaataatggaggtcttttttcaaaccgatggactattatttttagtgaaagagaataatattattggaaaaattattctatccacttattttatattggcagaatacaaagaatggtggggtccacgcggaacttgcggcccccgaaacctatcgagggcgcggtacccaagcaaggaacggaggaaacttgcggggcccgaaacctagtgataaaaggcatatcatccacacgatgaagatctgacggaccatatctcccaccacggaaagccatcagacgaggatcagacggccaggaaggaaccctagcgcccgccggtgccaatataaagcgccactctctctcgtcttaagccccagcctctccattcccctctccctctcgccgccgccgtctccttctcctactcccttcgaggtgtgttgttcatccgtcccgaatccatccatcccctcttcagatgtgttgttcatggctctaatagctctagatctgcttgtttgtgttgtttagctctagatctactcgcgcgcgcttctctctcgatctcctgtagaacaattttggttggttttttgtgcatatccatggtaattttgtctgcaatatggaggaggctttctaagctcctacgtagcatcgatctttagaattccctcggtttctgtttatttcttcgcgagggctctctgttatctgtaggagtagctgtaagcgcggttcgttacggattaatcgtcatgcttagttgaacctatcggtcgaaggatttgtgtgggttgtcgtgtagaattgacaccatctacttactgtactgatatgccgatctgtaggatactcttcattacttttgtttactgctagttgtggtgtagatttagcattctcaaacccatgctgtagcgtttctaatattgttacatagatctaccggtgcctgttaattgtattcgatcgggcgtttctacatctgtccgcccacctagttttatatgtggtaatcaaaattgcgttgacttcgtgatgctgtctgtgtactgtttttaatcgctcttacttagatgatcaacatggtgatggttacgatttactgttttctaatccctgttacttcgatgctgcagtttggatccatggaatcgggctccggcacggcggcagggtctggttatgtctatcggcagagcggaagcacccggtggaatccaacagcagaacagttgtcgctgctcaaggaactttattaccggaatggaattcggacaccgtcggcagatcaaattaggcaaatttcggcccggctgtccagatacggcaaaatagaagggaaaaacgtcttttactggtttcaaaatcataaagcacgggaacggcagaagaaaagactttccacggtcggctgcgaccctgctctcatagaaatgggtaacgtcgcgagcttggaatttgggaccgaaagcgctcttgaatctctcagctcaggcccgtccagcgagttgcgcgaggctcctacccgcaagttttatgagaagaaaaccgttggtgagaacagcaccataatcaatcctgttgagcagaactgcacactttcttgcggtacttcgcaggaatttcagtatgctgttgatagccgccgggtgatgaaggcaatggaagagaagcaagcaacggatgatgaaccggacggaaacaaatggacggagtcgaacaggcatgtgaagaccctccctcttttccccttgcataataatgaagatcagaccttgatcaagtcggacaaggaaatttattgccttgggagctgtgaaaaaaaaatggatctgtccccattgggacactcgggctctcagagggcgtcggcactggatttgtgcctgtctttgggtaatgaatcttgtggcctccacgacaattgaaagcttacgcgtgtcgactcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcga

SEQ ID NO: 51 - DNA, 70 bp, Zea mays
  source 1..70: mol_type = unassigned DNA
acgacttattatttgatttactcgtcacgattcccctctcctggtcgaacttttcaggtggggaaagctg

SEQ ID NO: 52 - DNA, 65 bp, synthetic construct
  misc_feature 1..65: note = Description of Artificial Sequence: Synthetic oligonucleotide
  misc_feature 1..65: note = target region from edited T0 plant (genotype A188) with a 5 bp deletion aligned to reference sequence of SEQ ID NO: 51 as shown in Fig. 21A
  source 1..65: mol_type = other DNA
acgacttattatttgatttactcgtcacgattctcctggtcgaacttttcaggtggggaaagctg

SEQ ID NO: 53 - DNA, 68 bp, synthetic construct
  misc_feature 1..68: note = Description of Artificial Sequence: Synthetic oligonucleotide
  misc_feature 1..68: note = target region from edited T0 plant (genotype B73) with a 2 bp deletion aligned to reference sequence of SEQ ID NO: 51 as shown in Fig. 21A
  source 1..68: mol_type = other DNA
acgacttattatttaatttactcgtcacgattcccctcctggtcgaacttttcaggtggggaaagctg

SEQ ID NO: 54 - DNA, 70 bp, Zea mays
  source 1..70: mol_type = unassigned DNA
cttattatttgatttactcgtcacgattcccctctcctggtcgaacttttcaggtggggaaagctgctgg

SEQ ID NO: 55 - DNA, 64 bp, synthetic construct
  misc_feature 1..64: note = Description of Artificial Sequence: Synthetic oligonucleotide
  misc_feature 1..64: note = target region from edited T0 plant (genotype A188) with a 6 bp deletion aligned to reference sequence of SEQ ID NO: 54 as shown in Fig. 21B
  source 1..64: mol_type = other DNA
cttattatttgatttactcgtcacgattccctggtcgaacttttcaggtggggaaagctgctgg

SEQ ID NO: 56 - DNA, 65 bp, synthetic construct
  misc_feature 1..65: note = Description of Artificial Sequence: Synthetic oligonucleotide
  misc_feature 1..65: note = target region from edited T0 plant (genotype B73) with a 5 bp deletion aligned to reference sequence of SEQ ID NO: 54 as shown in Fig. 21B
  source 1..65: mol_type = other DNA
cttattatttaatttactcgtcacgattcccctggtcgaacttttcaggtggggaaagctgctgg

SEQ ID NO: 57 - DNA, 70 bp, Zea mays
  source 1..70: mol_type = unassigned DNA
cgacttattatttgatttactcgtcacgattcccctctcctggtcgaacttttcaggtggggaaagctgc

SEQ ID NO: 58 - DNA, 62 bp, synthetic construct
  misc_feature 1..62: note = Description of Artificial Sequence: Synthetic oligonucleotide
  misc_feature 1..62: note = target region from edited T0 plant (genotype A188) with a 8 bp deletion aligned to reference sequence of SEQ ID NO: 57 as shown in Fig. 21C
  source 1..62: mol_type = other DNA
cgacttattatttgatttactcgtcacgattcccctcgaacttttcaggtggggaaagctgc

SEQ ID NO: 59 - DNA, 70 bp, Zea mays
  source 1..70: mol_type = unassigned DNA
cgacttattatttaatttactcgtcacgattcccctctcctggtcgaacttttcaggtggggaaagctgc